SEQUENCE LISTING (2 sequences)

SEQ ID NO: 1 - protein, 543 aa, Homo sapiens
MSSFSYEPYYSTSYKRRYVETPRVHISSVRSGYSTARSAYSSYSAPVSSSLSVRRSYSSSSGSLMPSLENLDLSQVAAISNDLKSIRTQEKAQLQDLNDRFASFIERVHELEQQNKVLEAELLVLRQKHSEPSRFRALYEQEIRDLRLAAEDATNEKQALQGEREGLEETLRNLQARYEEEVLSREDAEGRLMEARKGADEAALARAELEKRIDSLMDEISFLKKVHEEEIAELQAQIQYAQISVEMDVTKPDLSAALKDIRAQYEKLAAKNMQNAEEWFKSRFTVLTESAAKNTDAVRAAKDEVSESRRLLKAKTLEIEACRGMNEALEKQLQELEDKQNADISAMQDTINKLENELRTTKSEMARYLKEYQDLLNVKMALDIEIAAYRKLLEGEETRLSFTSVGSITSGYSQSSQVFGRSAYGGLQTSSYLMSTRSFPSYYTSHVQEEQIEVEETIEAAKAEEAKDEPPSEGEAEEEEKDKEEAEEEEAAEEEEAAKEESEEAKEEEEGGEGEEGEETKEAEEEEKKVEGAGEEQAAKKKD

SEQ ID NO: 2 - RNA, 3142 nt, Homo sapiens
  source 1..3142: mol_type = mRNA
gcacacagccatccatcctcccccttccctctctcccctgtcctctctctccgggctcccaccgccgccgcgggccggggagccaccggccgccaccatgagttccttcagctacgagccgtactactcgacctcctacaagcggcgctacgtggagacgccccgggtgcacatctccagcgtgcgcagcggctacagcaccgcacgctcagcttactccagctactcggcgccggtgtcttcctcgctgtccgtgcgccgcagctactcctccagctctggatcgttgatgcccagtctggagaacctcgacctgagccaggtagccgccatcagcaacgacctcaagtccatccgcacgcaggagaaggcgcagctccaggacctcaatgaccgcttcgccagcttcatcgagcgcgtgcacgagctggagcagcagaacaaggtcctggaagccgagctgctggtgctgcgccagaagcactccgagccatcccgcttccgggcgctgtacgagcaggagatccgcgacctgcgcctggcggcggaagatgccaccaacgagaagcaggcgctccagggcgagcgcgaagggctggaggagaccctgcgcaacctgcaggcgcgctatgaagaggaggtgctgagccgcgaggacgccgagggccggctgatggaagcgcgcaaaggcgccgacgaggcggcgctcgctcgcgccgagctcgagaagcgcatcgacagcttgatggacgaaatctcttttctgaagaaagtgcacgaagaggagatcgccgaactgcaggcgcagatccagtacgcgcagatctccgtggagatggacgtgaccaagcccgacctttccgccgcgctcaaggacatccgcgcgcagtacgagaagctggccgccaagaacatgcagaacgctgaggaatggttcaagagccgcttcaccgtgctgaccgagagcgccgccaagaacaccgacgccgtgcgcgccgccaaggacgaggtgtccgagagccgtcgtctgctcaaggccaagaccctggaaatcgaagcatgccggggcatgaatgaagcgctggagaagcagctgcaggagctggaggacaagcagaacgccgacatcagcgctatgcaggacacgatcaacaaattagaaaatgaattgaggaccacaaagagtgaaatggcacgatacctaaaagaataccaagacctcctcaacgtgaagatggctttggatattgagattgcagcttacaggaaactcttggaaggcgaggagacccgactcagtttcaccagcgtgggaagcataaccagtggctactcccagagctcccaggtctttggccgatctgcctacggcggtttacagaccagctcctatctgatgtccacccgctccttcccgtcctactacaccagccatgtccaagaggagcagatcgaagtggaggaaaccattgaggctgccaaggctgaggaagccaaggatgagcccccctctgaaggagaagccgaggaggaggagaaggacaaggaagaggccgaggaagaggaggcagctgaagaggaagaagctgccaaggaagagtctgaagaagcaaaagaagaagaagaaggaggtgaaggtgaagaaggagaggaaaccaaagaagctgaagaggaggagaagaaagttgaaggtgctggggaggaacaagcagctaagaagaaagattgaacccccatttccttaattatttcaggaataattctcccgaaatcaggtcaaccccatcaccaaccaaccaaccagttgagttccagattctatgtgaattaaaaagtcaatatatgtataattctgagatgacttaggttggacattcaatgttgtgctatgaatttcctctttatgcagagtatctgtttgcttgcagagtggctttctggcttgctgccagcctgtgcatggtccacgcttatgagttcaggatctacggcaatgtgaatcattcagatgtttacaataaaaaacaccacatgagtaaatgaattcactaatgttaatgttaaacttcatggaaaaatagtcctttgaaccttcggtggttagcaattaaagaccctgagttatgtgcaataaatagtaaataaagttataccgaatgatgtattttttgctgtggttgttacttaattaaaataccttaaagatggcaccaatataaagtatataccagtggactattgacctccaattttttaaaaagttgaaattttaacaattaccaatacttttttttcttccttcaattggaaattctgagggatacagttatgtcatgattacttgtgaaatctctccccacaaaaattttactgataataaacatgagtaaatgagaaagtctataaataacaatacactttgcctcataacacgtgttgtgttaggttttgggatggctttctagggagcaattctgaaattacttgacagatacactctttatggtaaacaggtcatttcagagtctagagtgctaaccattacactatggaaccatgataaataggtcatttcaaactgatatgtcacaatgcaatatggatagaatatgaatcccatacattcatccctataactgcattttaaaatatagtctaaatatcatgggaggaggttaaaacctgagaacatggtacaataagaaggaataattttttatctatatataatttcaggagaagggattaaaactaccaaaggttatttgccactccactgcaaacatgttgcctgcaatcctgagctcatgatttagtgtgttgttaagcgtaattctgttgcctcaagtaaataccacttaatgctacacatttttaaacctttaaaagctacagacacaagtaatgaaattatgagtcacttaaggggggaggtaatatgcttgtatgttacattgacattgctctgcatgtctctactgggaaatatttggatttcaatgatagcttgactggttggacattgcatcaaatacacgccccatgtatcctttctgtcgtaaagttaagaacgctcttgcatttgatgtggaataaatatagatgatattacaaaaaaaaaaaaaaaaaaaaaaaaaaaaaa

(19) United States
(12) Patent Application Publication
(10) Pub. No.: US 2026/0287580 A1
(43) Pub. Date: Sep. 24, 2026

(54) High-sensitivity Assay for Serum Neurofilament Light Chain

(71) Applicant: Siemens Healthcare Diagnostics Inc., Tarrytown, NY (US)

(72) Inventors: Eddine Merabet, White Plains, NY (US); James Freeman, Sandy Hook, CT (US); Sarmistha Ray, Yorktown Heights, NY (US); Arejas James Uzgiris, Berkeley, CA (US)

(73) Assignee: Siemens Healthcare Diagnostics Inc., Tarrytown, NY (US)

(21) Appl. No.: 19/473,643

(22) PCT Filed: Apr. 26, 2024
(86) PCT No.: PCT/US2024/026599

§ 371 Date: Oct. 8, 2025

Related U.S. Application Data
(60) Provisional application No. 63/498,806, filed on Apr. 27, 2023.
(60) Provisional application No. 63/588,465, filed on Oct. 6, 2023.

(51) Int. Cl. G01N 33/543; C09K 11/07; G01N 21/76; G01N 33/68

(52) U.S. Cl. CPC G01N 33/54313; C09K 11/07; G01N 21/76; G01N 33/54353; G01N 33/6896; G01N 2470/06

(57) ABSTRACT

The present disclosure provides methods and kits for identifying and treating individuals at risk of or suffering from disease such as a neurological condition or disease. In general, detection or measurement of one or more biomarkers, such as neurofilament light chain (NfL), and combinations thereof with assays of the present disclosure, assists in the identification of disease such as neurological disease. The present disclosure also provides methods for selecting patients for treatment of neurological disease.

### CROSS REFERENCE TO RELATED APPLICATIONS

This application claims priority to and the benefit of U.S. App. No. 63/498,806, filed Apr. 27, 2023 and U.S. App. No. 63/588,465, filed Oct. 6, 2023, each of which are incorporated by reference in their entirety.

### SEQUENCE LISTING

The present application contains a Sequence Listing which has been submitted electronically in XML format. The Sequence Listing having filename 2023P07984US-Sequence_Listing.xml, 6,442 bytes in size and created on Apr. 26, 2024, is hereby incorporated by reference in its entirety.

### TECHNICAL FIELD

The present disclosure relates generally to the fields of molecular biology and the health of a subject.

### BACKGROUND

Neurofilaments are cytoskeleton proteins that are key scaffolding proteins of neurons and are primarily located in the axons (See e.g., Sen M K, Hossain M J, Mahns D A, Brew B J. Validity of serum neurofilament light chain as a prognostic biomarker of disease activity in multiple sclerosis. J Neurol. April 2023; 270(4):1908-1930. doi:10.1007/s00415-022-11507-y). The subunits that include neurofilaments are categorized based on size and include neurofilament light chain (NfL), medium, and neurofilament heavy chain. Following axonal damage, NfL is released into cerebrospinal fluid (CSF) and, to a lesser extent, into blood circulation (See e.g., Yang J, Hamade M, Wu Q, et al. Current and Future Biomarkers in Multiple Sclerosis. Int J Mol Sci. May 24, 2022; 23(11). doi:10.3390/ijms23115877 and FIG. 6 below). As a result, research has been focused on the use of NfL as a biomarker for degenerative and neurological (See e.g., Disanto G, Barro C, Benkert P, et al. Serum Neurofilament light: A biomarker of neuronal damage in multiple sclerosis. Ann Neurol. June 2017; 81(6):857-870. doi:10.1002/ana.24954). While NfL levels in CSF are generally 10-40 times higher than in blood, obtaining such a sample is a relatively invasive procedure which limits the potential utility of CSF NfL (cNfL) in routine clinical settings. Several studies have also demonstrated that peripheral NfL levels correlate closely with cNfL and axonal damage and neurodegeneration. What are needed are highly sensitive assays that allow non-invasive and reliable measurement of NfL in serum (sNfL) for clinical analysis.

NfL is a neuronal cytoplasmic protein highly expressed in myelinated axons and implicated in amyotrophic lateral sclerosis, multiple sclerosis, Alzheimer's disease, and Huntington's disease. However, NfL concentration in blood is limited due, in part, to the necessity of its crossing the blood brain barrier. Due to the very low concentrations of neurofilament light chain (NfL) in blood (as it is necessary for this analyte to cross the blood brain barrier), analytical methods do not contain the requisite resolution to identify its presence in blood or, to track changes in NfL concentration associated with disease progression.

Prior art of interest includes: WO2015066211 A1, WO2019199865A1, WO2019199869A1, WO2019199871A1, US 2017/0160292A1, US 2017/0044264, US 2015/0185232A1, and US 2014/0086836A1 (all of which are entirely incorporated herein by reference). Additional prior art of interest includes KORLEY et al. “Performance Evaluation of a Multiplex Assay for Simultaneous Detection of Four Clinically Relevant Traumatic Brain Injury Biomarkers,” Journal of Neurotrauma, 14 Dec. 2018 (14.12.18), Vol. 36, Iss. 1, Pgs. 182-197 which is entirely incorporated herein by reference.

Typically, the clinical laboratory defines lower limits of most assays as the lower limit of quantitation (LLOQ) or limit of detection (LOD). Lower limit of quantitation refers to the lowest value at which an accurate quantitative value (CV<20%) can be reported, while the LOD is the value at which the assay is able to distinguish between the presence or absence of an analyte. Certain analytes having low concentration such as NfL cannot be measured with most immunoassays due to lower limits of detection intrinsic in typical immunoassay formats.

Accordingly, there is a need in the art for compositions, kits, and methods including non-invasive and reliable measurement of NfL in serum (sNfL) for clinical analysis, with an acceptable level of specificity and/or in a manner that aids patient compliance.

### SUMMARY

The present disclosure provides compositions, kits, and methods for detecting and/or identifying neurofilament light chain (NfL), and highly sensitive assay embodiments that allow non-invasive and reliable measurement of NfL in serum (sNfL) such as for clinical analysis. Further, the present disclosure provides methods of classifying neurological disease patients compared to normal individuals. The present disclosure further recognizes that these biomarkers (e.g., NfL) in compositions, kits, and methods can be useful for classifying, detecting and/or diagnosing disease (such as neurological) without the need for performing invasive or costly tests. This represents a significant advancement in patient care, as disease resulting can be detected and identified by methods that are more comfortable for the patient, inflict less harm to the patient, and/or decrease the amount of time a patient needs to recover following the detection and/or diagnostic method.

In addition, the present disclosure provides that certain biomarkers (e.g., NfL) are useful in detecting disease with improved sensitivity. The present disclosure provides that certain biomarkers (e.g., NfL) are useful in detecting disease with improved selectivity.

The increased sensitivity achieved with biomarkers (e.g., NfL) described herein decreases the number of false negatives obtained when detecting and/or identifying a neurological disease. The decrease in false negatives will, in turn, help to ensure that more disease patients receive earlier treatments critical for reducing signs, symptoms, and conditions associated with disease, as well as promoting long-term survival of disease patients.

The present disclosure also includes methods for the detection of analytes in a sample through the use of acridinium conjugates to analytes or binding partners for analytes such as antibodies. These acridinium conjugates are typically provided on a carrier protein such as bovine serum albumin (e.g., in excess), which is linked to the analyte or binding partner thereof. These conjugates are typically able to provide the required chemiluminescent output over a range of standard concentrations in an assay (such as a sandwich assay format) to result in the required resolution for analytes that may be present at low concentrations in biological samples.

Additionally, preparation of the conjugates may occur in a specific assay format suitable for characterization with the required resolution. For example, the RLU slope factor (for between 0 μg/mL and 660 μg/mL) associated with the conjugate and/or assay may be greater than (or up to 2000) 500 or greater than 600. The present disclosure is partially premised on the discovery that slope factors for an immunoassay between 0 μg/mL and 660 μg/mL are possible using the immunoassays described herein and that such slope factor may be required for clinical utility of the detection of certain analytes. In some embodiments, the sample is blood, saliva, or serum. In some embodiments, the sample derived from a biological sample such as a diluted biological sample (e.g., as mixed with saline). In some embodiments, the analyte is an analyte that crosses the blood brain barrier into the blood (e.g., a neuronal analyte such as a neurofilament light chain) and the sample is blood. In some embodiments, the analyte is a serum analyte (e.g., serum neurofilament biomarker such as serum neurofilament light chain (sNfL)) and the biological sample is blood.

In certain implementations, the chemiluminescent label is conjugated to a first immunoglobulin antibody fragment (e.g., F(ab), F(c)). In particular embodiments, the chemiluminescent label is conjugated to F(ab) fragments which may still bind to antigens or analytes but is monovalent (and without the F(c) portion). In some embodiments, the first antibody or antibody fragment is a mouse monoclonal antibody or fragment thereof (e.g., F(ab)). In some embodiments, the linker between the antibody or fragment thereof and the carrier protein comprises (or is) polyethylene glycol (PEG) which may be independently covalent linked to both moieties. For example, the polyethelene glycol linker may have from 2-20 (e.g., 2-10, 2-5) ethylene glycol units. In some embodiments, the carrier protein is Keyhole Limpet Hemocyanin (KLH), Bovine Serum Albumin (BSA), or cationized BSA. By leveraging the properties of the carrier protein, more labelling compounds may be present in the conjugate. For example, in some embodiments, the ratio of acridinium to carrier protein is from 50:1 to 1:1 (e.g., from 30:1 to 1:1, from 25:1 to 5:1) by weight.

The present disclosure provides a method that includes receiving, by a processor of a computing device, biomarker profile including data corresponding to a level of one or more biomarkers in a sample such as serum from a subject, wherein the one or more biomarkers include neurofilament light chain (NfL). The method may include providing, by the processor, input to a machine-learned algorithm, the input including the biomarker profile. The method may include determining, by the processor, a biomarker score for the subject with the machine-learned algorithm based on the input. In embodiments, the methods include obtaining a signal from an assay embodiment of the present disclosure.

The present disclosure also provides a method that includes receiving, by a processor of a computing device, biomarker profile including data corresponding to a level of one or more biomarkers in a sample from a subject, wherein the one or more biomarkers include neurofilament light chain (NfL). The method may include providing, by the processor, input to a machine-learned algorithm, the input including the biomarker profile. The method may include determining, by the processor, whether the subject is at risk of or has (e.g., is suffering from) a disease such as a neurological condition, neurological disease, or injury to the brain or central nervous system (such as TBA) with the machine-learned algorithm based on the input. In some embodiments, determining whether the subject is at risk of or has (e.g., is suffering from) neurological disease or condition with the machine-learned algorithm includes determining, by the processor, a probability that the subject has neurological disease.

In some embodiments of methods disclosed herein, the method includes receiving, by the processor, data corresponding to one or more imaging-based biomarkers for the subject; and providing, by the processor, the input to the machine-learned algorithm, wherein the input includes the data corresponding to the imaging-based biomarkers. In embodiments, data is obtain from a signal generated by an assay of the present disclosure.

In some embodiments of methods disclosed herein, a method includes determining, by the processor, if the subject is at risk of or has (e.g., is suffering from) a neurological disease or neurological condition with the machine-learned algorithm [e.g., wherein an output of the machine-learned algorithm is a determination of whether the subject is at risk of or has (e.g., is suffering from) neurological disease]. In embodiments, data is obtained from a signal generated by an assay of the present disclosure.

In some embodiments of methods disclosed herein, a method includes classifying, by the processor, the subject as having or not having neurological disease or condition with the machine-learned algorithm. In some embodiments of methods disclosed herein, the machine-learned algorithm is a classifier for neurological disease. In some embodiments of methods disclosed herein, the classifier or the classifying has a sensitivity and a specificity each of more than 80% (e.g., at least one or both of more than 90%). In embodiments, data is obtained from a signal generated by an assay of the present disclosure.

In some embodiments of methods disclosed herein, the subject is a human subject. In some embodiments of methods disclosed herein, the sample includes blood, serum, plasma, central nervous system tissue, or combinations thereof.

The present disclosure also provides a method that includes receiving, by a processor of a computing device, data corresponding to one or more biomarkers for a subject, wherein the one or more biomarkers include NfL or serum NfL. The method may include providing, by the processor, input to a machine-learned algorithm, the input including the data. The method may include determining, by the processor, with the machine-learned algorithm, at least one of (i) a neurological disease state for the subject, (ii) whether the subject has or does not have a neurological disease, disorder, or condition, or (iii) a probability that the subject has a neurological disease, disorder or condition.

The present disclosure also provides a method that includes detecting a level of neurofilament light chain (NfL) in a first serum sample taken from a subject at a first time and in a second serum sample taken from the subject at a second time that is after the first time. The method may include comparing the level of NfL in the second sample to the level of NfL in the first sample.

In some embodiments, the method includes detecting a level of neurofilament light chain (NfL) in a third sample taken from the subject at a third time that is after the first time and after the second time. In some embodiments, the method includes comparing the level of NfL in the third sample to at least the level of NfL in the second sample (e.g., and the first sample).

In some embodiments, the first time and the second time (e.g., and the third time) are separated by at least a month (e.g., at least three months, at least 6 months, or at least one year).

In some embodiments, the method includes determining a biomarker score based on the comparison between the level of NfL in the second sample to the level of NfL in the first sample.

In some embodiments, the method includes determining whether the subject is at risk of or has (e.g., is suffering from) neurological disease or condition based on the comparison between the level of NfL in the second sample to the level of NfL in the first sample [e.g., and the comparison between the level of NfL in the third sample to the level of NfL in the second sample (e.g., and optionally also to the level of NfL in the first sample)][e.g., as compared to a change in level of NfL over a similar period of time for a control subject (e.g., at a similar age (e.g., within three years))], using the assay of the present disclosure. In some embodiments, the method includes determining that the subject is at risk of or has neurological disease based on the level of NfL in the second sample exceeding the level of NfL in the first sample. In some embodiments, determining that the subject is at risk of or has neurological disease is further based on age of the subject. In some embodiments, determining that the subject is at risk of or has neurological disease is based on the level of NfL in the second sample exceeding the level of NfL in the first sample by a threshold amount. In some embodiments, determining that the subject is at risk of or has neurological disease is based on the level of NfL in the second sample exceeding the level of NfL in the first sample by at least 5%, at least 10%, at least 20%, at least 25%, at least 30%, at least 40%, at least 50%, at least 60%, at least 70%, at least 80%, at least 90%, at least 100%, at least 125%, at least 150%, at least 175%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, or at least 1000% of the level of NfL in the first sample (e.g., wherein that amount is the threshold amount), and wherein the amount is determined using the assay of the present disclosure. In embodiments, the assay of the present disclosure is an IVD assay.

In some embodiments, the first sample and the second sample (e.g., and the third sample) are serum samples.

Methods for the detection or quantification of an analyte in a sample (e.g., a biological sample such as blood, saliva, serum, a sample derived from a biological sample such as a diluted biological sample) are provided. These methods may comprise:
(a) mixing the sample with a composition comprising a chemiluminescent label conjugated to a first antibody or antibody fragment that binds to the analyte, wherein the chemiluminescent label is bound to a carrier protein comprising a linker, and the linker is bound to the antibody or antibody fragment,
(b) adding, to the mixture, a particle having a second antibody or antibody fragment that binds to the analyte attached to the surface of the particle; (c) preparing the mixture to measure chemiluminescence (e.g., by separation of the particle having chemiluminescent acridiniums conjugated to the surface);
(d) triggering chemiluminescence from the preparation; and
(e) detecting the presence or calculating the concentration of said at least one analyte by comparing the amount of chemiluminescence with a standard dose response curve which relates the amount of light emitted to a known concentration of the at least one of the multiple analytes.

In some embodiments, the analyte is a neurofilament such as neurofilament light chain (e.g., serum neurofilament light chain). In particular embodiments, the sample is blood.

The method may be used to characterize a disease, disorder, or condition. For example, if the analyte is a neuronal biomarker such as neurofilament light chain, a subject from which the sample is taken may be diagnosed with a neuronal disease (e.g., amyotrophic lateral sclerosis, multiple sclerosis, Alzheimer's disease, and Huntington's disease) if the analyte level is above a certain concentration in the blood (e.g., between 10 μg/mL-20 pg/mL). In some embodiments, the method provides the requisite resolution to track progression of the disease, disorder, or condition. For example, the concentration of the analyte may be measured from a first sample taken from a subject. After a time period, a second biological sample may be taken from the subject and the concentration may be determined. Changes in the biomarker level may be correlated with disease progression.

The present disclosure also provides a method that includes detecting a level of neurofilament light chain (NfL) in a series of samples taken from a subject using the assay of the present disclosure. In some embodiments, the method includes determining change in the level of NfL over a period of time from the series of samples.

In some embodiments, the series of samples have been taken periodically. In some embodiments, successive samples in the series have been taken monthly, quarterly, biannually (every six months), annually, every two years, every three years, or every five years. In some embodiments, successive samples in the series have been taken no more recently together than monthly, quarterly, biannually (every six months), annually, every two years, every three years, or every five years.

In some embodiments, the samples are blood samples (e.g., have been taken by blood draw). In some embodiments, the method includes taking the series of samples from the subject.

In some embodiments, the period of time is at least one month (e.g., at least three months, at least six months, at least one year, at least two years, at least three years, at least four years, at least five years, at least six years, at least ten years, or at least fifteen years).

In some embodiments, the determining step includes determining that the level did not change over time or that the level changed a negligible amount (e.g., to a degree not indicative of the subject having neurological disease).

In some embodiments, the method includes determining a biomarker score based on the comparison between the level of NfL in the second sample to the level of NfL in the first sample.

The assays of the present disclosure are typically a 2-step sandwich immunoassay using acridinium ester chemiluminescent technology. The assay may employ two anti-sNfL antibodies. The first antibody, in the Lite Reagent, may be a mouse monoclonal anti-sNfL antibody labeled with acridinium ester. The second antibody may be a biotinylated mouse monoclonal anti-sNfL antibody bound to streptavidin-coated paramagnetic microparticles in the Solid Phase. The required sensitivity of may be achieved by 1) signal amplification (e.g., amplification achieved by the use acridinium ester conjugation to the carrier protein-particularly specific excesses of acridiniums on the carrier protein), 2) decrease non-specific binding (e.g., decrease through the use of antibody fragments linked to the carrier protein) and 3) timing of the reagent additions in the analyzer (e.g., addition of the Lite Reagent prior to addition of the Solid Phase, incubation time periods and intensity associated with each addition).

The present disclosure also provides a method including administering a therapeutically effect amount of a neurological medication or neurological therapy to a subject that has been diagnosed with neurological disease or condition based on a change in level of NfL for the subject over a period of time. In some embodiments, the period of time is at least one month (e.g., at least three months, at least six months, at least one year, at least two years, at least three years, at least four years, at least five years, at least six years, at least ten years, or at least fifteen years).

The present disclosure also provides a method that includes detecting a level of neurofilament light chain (NfL) in a first sample taken from a subject at a first time and in a second sample taken from the subject at a second time that is after the first time.

The present disclosure also provides a method that includes monitoring a level of NfL for a subject over a period of time. The period of time may be at least one month (e.g., at least three months, at least six months, at least one year, at least two years, at least three years, at least four years, at least five years, at least six years, at least ten years, or at least fifteen years).

In some embodiments of methods disclosed herein, the method accounts for a neurological condition of the subject (e.g., and control subject, if used). For example, the method may account for at least one neurological disease, disorder, or condition that the subject has or does not have. In some embodiments a threshold amount is set based on the neurological condition of the subject. In some embodiments, using a change in level of NfL of the subject over a period of time (e.g., between a first sample and a first time and a second sample at a second time that is after the first time) to determine whether the subject is at risk of having or has neurological disease includes considering the neurological condition of the subject. In some embodiments, a neurological condition of a subject is considered by determining that the subject has or is at risk of having neurological disease when there is a bigger change in the level of NfL over the period of time than if the subject did not have the neurological disease, disorder, or condition.

The present disclosure also provides a non-transitory computer readable medium containing executable instructions that when executed cause a processor to perform operations including a method disclosed herein. In embodiments, data is obtain from a signal generated by an assay of the present disclosure.

The present disclosure also provides a system including a non-transitory computer readable medium and a processor, wherein the non-transitory computer readable medium has executable instructions stored thereon that, when executed by the processor, perform operations including a method disclosed herein. In some embodiments, the computer readable medium further has a (e.g., the) machine-learned algorithm stored thereon (e.g., wherein the machine-learned algorithm performs at least one of the operations of a method disclosed herein). In embodiments, data is obtain from a signal generated by an assay of the present disclosure.

The chemiluminescent label conjugated to the first antibody or antibody fragment that binds to the analyte may be formed by
i) reacting a chemiluminescent acridinium compound comprising a reactive functional group with the carrier protein to label the carrier protein; and
ii) reacting a linking compound (e.g., a compound comprising a linker with reactive functional groups on each end), the first antibody or antibody fragment, and the labelled carrier protein.

In specific embodiments, the chemiluminescent acridinium comprising a reactive functional group is TSPAE-NHS:

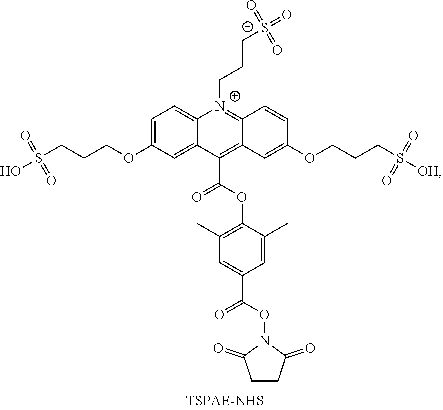

or a salt thereof.

The assay formats described herein may involve the presence of a solid phase conjugated to the second antibody or fragment thereof. For example, the second antibody or antibody fragment may be a biotinylated antibody or antibody fragment (e.g., and the solid phase is coated with streptavidin).

By using the conjugates and assay formats described herein, high resolution assays may be performed. These assays may be performed on analytes typically having previously thought undetectable concentrations in biological samples such as those analytes that cross the blood brain barrier. Specific incubation and sequential steps as described herein may result in these assay formats with increased resolution. For example, the detecting step may be able detect a difference in concentration of the analyte of less than 5 μg/mL (e.g., less than 4 μg/mL, from 1 μg/mL to 5 μg/mL, from 2-5 μg/mL, from 2-4 μg/mL, from 3-4 μg/mL, from 2-3 μg/mL).

In some embodiments, the preparing step may comprise separating the particle (complexed with the label) from the mixture and the chemiluminescence is triggered from the particle or the separated mixture. In some embodiments, the method further comprises incubating the mixture prior to adding the particle. In some embodiments, the incubating prior to addition of the solid phase comprises heating the mixture for more than 30 minutes (e.g., from 30 minutes to 120 minutes, from 30 minutes to 60 minutes). In some embodiments, the method further comprises incubating the mixture after adding the particle. In some embodiments, this incubation after particle addition may involve heating the mixture for less than 30 minutes (e.g., from 10 minutes to 30 minutes, from 10 minutes to 20 minutes). In certain implementations, the labeled conjugate is first added to the biological sample, optionally incubated, then the solid particle is added to the biological sample/labelled conjugate mixture, which is optionally incubated a second time.

Immunoassay compositions are also provided. These compositions typically comprise a chemiluminescent label conjugated to a first antibody fragment that binds to an analyte (e.g., neurofilament light chain), wherein the chemiluminescent label is bound to a carrier protein comprising a linker (e.g., polyethylene glycol such as PEG₂-PEG₁₅ such as PEG₄), and the linker is bound to the antibody fragment; and a carrier or excipient. In some embodiments, the composition further comprises a buffer. In various implementations, the composition comprises one or more of a protease inhibitor, surfactant, preservative, blocker, and/or a heterophilic antibody blocker. In particular embodiments, the carrier protein is bovine serum albumin. In some embodiments, the weight ratio of said chemiluminescent acridinium to carrier protein is from 50:1 to 1:1 (e.g., from 30:1 to 1:1, from 25:1 to 5:1) by weight. The reagent may comprise a concentration of each detectable conjugate of from 10 to 30 ng/mL. Reagents of the present disclosure include compositions comprising the indicated components, and optionally an excipient, carrier, or solvent. The reagents of the present disclosure may include a surfactant.

Kits for the detection of an analyte are also provided. The kit may comprise an immunoassay reagent composition of the present disclosure in a container suitable for use with an analyzer.

Solid particles are also provided. These solid particles may be formed during the course of the measurement assay (and are typically used for subsequent analysis). The solid particle may be coated with streptavidin conjugated to biotinylated mouse antibody (e.g., anti neurofilament) optionally through a linker (e.g., PEG, Iodo-PEG), wherein the anti neurofilament antibody is bound to an analyte (e.g., neurofilament), and
the bound analyte is further bound to a monoclonal mouse antibody fragment linked to a carrier protein conjugated to one or more chemiluminescent acridiniums. In various implementations, the monoclonal mouse antibody fragment is linked to the carrier protein through a linker (e.g., PEG).

Following addition of the two sets of chemiluminescent labels, the sample is typically prepared to induce chemiluminesence in a manner that the analytes can be measured and/or their concentration quantified. For example, the preparing step may comprise:
(e1) providing a solid support having immobilized thereon a molecule capable of forming a binding complex with said at least one analyte and capable of forming a binding complex with a chemiluminescent label in the first set and/or the second set of chemiluminescent labels; and
(e2) separating said solid support from said mixture.

Any two or more of the features described in this specification, including in this summary section, may be combined to form implementations of the disclosure, whether specifically expressly described as a separate combination in this specification or not. These and other aspects of the invention will be better understood by reference to the following detailed description including the appended claims.

### BRIEF DESCRIPTION OF THE DRAWINGS

FIG. 1 depicts an exemplary block diagram of a computer system 1100.

FIG. 2 depicts an exemplary flow chart of a method 1200.

FIG. 3 depicts an exemplary flow chart of a method 1300.

FIG. 4 is a block diagram of an example of a network environment 2400 for use in the methods and systems described herein.

FIG. 5 is a block diagram of an example of a computing device and an example mobile computing device.

FIG. 6 depicts axonal damage causes release of NfL into CSF and blood.

FIG. 7 depicts ATELLICA IM brand chemical analyzer sNfL assay architecture.

FIG. 8 depicts low-end precision profile of the ATELLICA brand sNfL assay of the present disclosure suitable for use in embodiments of the present disclosure.

FIG. 9 depicts weighted least squares regression of measured means vs expected values for evaluation of linearity.

FIG. 10 depicts sample equivalency comparison between sNfL in serum vs EDTA plasma.

FIG. 11 depicts correlation of ATELLICA sNfL Assay with a different nFI assay.

FIG. 12 depicts an assay embodiment of the present disclosure.

FIG. 13 shows the SDS-PAGE gel for the neurofilament antibody digestion.

FIG. 14A provides a schematic of an exemplary binding complex formed from a sandwich assay of the present disclosure. FIG. 14B provides an exemplary high resolution assay schematic.

FIG. 15 provides the RLU measured at each standard concentration for a high resolution assay (slope factor greater than 600) of the present disclosure.

FIG. 16 is a table illustrating the assay parameters and measured outputs for various assays having slope factors less than 600.

The figures are not necessarily to scale.

### Definitions

For convenience, certain terms employed in the specification, including the examples and appended claims, are collected here. Unless defined otherwise, all technical and scientific terms used herein have the same meaning as commonly understood by one of ordinary skill in the art to which this disclosure pertains.

Unless otherwise explicitly defined, the following terms and phrases are intended to have the following meanings throughout this disclosure.

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Such polypeptide may be naturally produced (e.g., generated by an organism reacting to an antigen), or produced by recombinant engineering, chemical synthesis, or other artificial system or methodology. Exemplary antibody agents include, but are not limited to, human antibodies, primatized antibodies, chimeric antibodies, bi-specific antibodies, humanized antibodies, conjugated antibodies (e.g., antibodies conjugated or fused to other proteins, radiolabels, cytotoxins), Small Modular ImmunoPharmaceuticals (“SMIPs™”), single chain antibodies, cameloid antibodies, and antibody fragments. As used herein, the term “antibody agent” also includes intact monoclonal antibodies, polyclonal antibodies, single domain antibodies (e.g., shark single domain antibodies (e.g., IgNAR or fragments thereof)), multispecific antibodies (e.g. bi-specific antibodies) formed from at least two intact antibodies, and antibody fragments so long as they exhibit the desired biological activity. An antibody agent can have antibody constant region sequences that are characteristic of mouse, rabbit, primate, or human antibodies. In some embodiments, the term encompasses stapled peptides. In some embodiments, the term encompasses one or more antibody-like binding peptidomimetics. In some embodiments, the term encompasses one or more antibody-like binding scaffold proteins. In some embodiments, the term encompasses monobodies or adnectins. In many embodiments, an antibody agent is or includes a polypeptide whose amino acid sequence includes one or more structural elements recognized by those skilled in the art as a complementarity determining region (CDR); in some embodiments an antibody agent is or includes a polypeptide whose amino acid sequence includes at least one CDR (e.g., at least one heavy chain CDR and/or at least one light chain CDR) that is substantially identical to one found in a reference antibody. In some embodiments, an antibody agent is or includes a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain. In some embodiments, an antibody agent may contain a covalent modification (e.g., attachment of a glycan, a payload (e.g., a detectable moiety, a therapeutic moiety, a catalytic moiety, etc.), or other pendant group (e.g., poly-ethylene glycol, etc.).

Biomarker: The term “biomarker” or “biological marker” is used herein, consistent with its use in the art, to refer to an entity whose presence, level, or form, correlates with a particular biological event or state of interest, so that it is considered to be a “marker” of that event or state. To give but a few examples, in some embodiments, a biomarker may be or include a marker for a particular disease state, or for likelihood that a particular disease, disorder or condition may develop, occur, or reoccur. In some embodiments, a biomarker may be or include a marker for a particular disease or therapeutic outcome, or likelihood thereof. Thus, in some embodiments, a biomarker is predictive, in some embodiments, a biomarker is prognostic, in some embodiments, a biomarker is diagnostic, of the relevant biological event or state of interest. In some embodiments, a biomarker is a possible biomarker of the relevant biological event or state of interest. A biomarker may be an entity of any chemical class. For example, in some embodiments, a biomarker may be or include a nucleic acid, a polypeptide, a small molecule, or a combination thereof. In some embodiments, a biomarker is a cell surface marker. In some embodiments, a biomarker is intracellular. In some embodiments, a biomarker is found in a particular tissue (e.g., neurological tissue such as brain or CNS tissue). In some embodiments, a biomarker is found outside of cells (e.g., is secreted or is otherwise generated or present outside of cells, e.g., in a body fluid such as blood, urine, tears, saliva, cerebrospinal fluid, etc.

Characteristic fragment: The term “characteristic fragment” refers to a fragment of a biomarker (e.g., NfL) that is sufficient to identify the biomarker from which the fragment was derived. For example, in some embodiments, a “characteristic fragment” of a biomarker is one that contains an amino acid sequence, or a collection of amino acid sequences, that together allow for the biomarker from which the fragment was derived to be distinguished from other possible biomarkers, proteins, or polypeptides. In some embodiments, a characteristic fragment includes at least 10, at least 20, at least 30, at least 40, or at least 50 amino acids.

Gene product or expression product: As used herein, the term “gene product” generally refers to an RNA transcribed from the gene (pre- and/or post-processing) or a polypeptide (pre- and/or post-modification) encoded by an RNA transcribed from the gene.

Hybridization: The term “hybridization” refers to the physical property of single-stranded nucleic acid molecules (e.g., DNA or RNA) to anneal to complementary nucleic acid molecules. Hybridization can typically be assessed in a variety of contexts—including where interacting nucleic acid molecules are studied in isolation or in the context of more complex systems (e.g., while covalently or otherwise associated with a carrier entity and/or in a biological system or cell). In some embodiments, hybridization can be detected by a hybridization technique, such as a technique selected from the group consisting of in situ hybridization (ISH), microarray, Northern blot, and Southern blot. In some embodiments, hybridization refers to 100% annealing between the single-stranded nucleic acid molecules and the complementary nucleic acid molecule. In some embodiments, annealing is less than 100% (e.g., at least 95%, at least 90%, at least 85%, at least 80%, at least 75%, at least 70% of a single-stranded nucleic acid molecule anneals to a complementary nucleic acid molecule). Hybridization techniques, and methods for evaluating hybridization, are well known in the art. See, e.g., Sambrook, et al., 1989, Molecular Cloning: A Laboratory Manual, Second Edition, Cold Spring Harbor Press, Plainview, N.Y. Those skilled in the art understand how to estimate and adjust the stringency of hybridization conditions such that sequences having at least a desired level of complementary will stably hybridize, while those having lower complementary will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J.

Detection agent: The term “detection agent” as used herein refers to any element, molecule, functional group, compound, fragment or moiety that is detectable. In some embodiments, a detection agent is provided or utilized alone. In some embodiments, a detection agent is provided and/or utilized in association with (e.g., joined to) another agent. Examples of detection agents include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴CU, ¹⁸⁷Re, ¹¹¹n, ⁹⁰Y, ^{99m}Tc, ¹⁷⁷Lu, ⁸⁹Zr etc.), fluorescent dyes, chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes, colorimetric labels (such as, for example, dyes, colloidal gold, and the like), biotin, dioxigenin, haptens, and proteins for which antisera or monoclonal antibodies are available.

Diagnostic test: As used herein, “diagnostic test” is a step or series of steps that is or has been performed to attain information that is useful in determining whether a patient has a disease, disorder or condition and/or in classifying a disease, disorder or condition into a phenotypic category or any category having significance with regard to prognosis of a disease, disorder or condition, or likely response to treatment (either treatment in general or any particular treatment) of a disease, disorder or condition. Similarly, “diagnosis” refers to providing any type of diagnostic information, including, but not limited to, whether a subject is likely to have or develop a disease, disorder or condition, state, staging or characteristic of a disease, disorder or condition as manifested in the subject, information related to the nature or classification of a tumor, information related to prognosis and/or information useful in selecting an appropriate treatment or additional diagnostic testing. Selection of treatment may include the choice of a particular therapeutic agent or other treatment modality such as surgery, radiation, etc., a choice about whether to withhold or deliver therapy, a choice relating to dosing regimen (e.g., frequency or level of one or more doses of a particular therapeutic agent or combination of therapeutic agents), etc. Selection of additional diagnostic testing may include more specific testing for a given disease, disorder, or condition.

Sample: As used herein, the term “sample” refers to a biological sample obtained or derived from a human subject, as described herein. In some embodiments, a biological sample includes biological tissue or fluid. In some embodiments, a biological sample may include blood; blood cells; tissue or fine needle biopsy samples; cell-containing body fluids; free floating nucleic acids; cerebrospinal fluid; lymph; tissue biopsy specimens; surgical specimens; other body fluids, secretions, and/or excretions; and/or cells therefrom. In some embodiments, a biological sample includes cells obtained from an individual, e.g., from a human or animal subject. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood). In some embodiments, a sample is CNS tissue obtained from the subject. In some embodiments, as will be clear from context, the term “sample” refers to a preparation that is obtained by processing (e.g., by removing one or more components of and/or by adding one or more agents to) a primary sample. For example, filtering using a semi-permeable membrane. As another example of sample processing, the sample may be a plasma sample that is treated with an anticoagulant selected from the group consisting of EDTA, heparin, and citrate. As another example of sample processing, the sample may be processed to isolate one or more proteins (e.g., by capturing proteins with one or more antibodies). A “processed sample” may include, for example, nucleic acids or polypeptides extracted from a sample or obtained by subjecting a primary sample to techniques such as amplification or reverse transcription of mRNA, isolation and/or purification of certain components.

Subject: As used herein, the term “subject” refers to an organism, for example, a mammal (e.g., a human). In some embodiments a human subject is an adult, adolescent, or pediatric subject. In some embodiments, a subject is at least 50, at least 55, at least 60, at least 65, at least 70, at least 75, or at least 80 years of age. In some embodiments, a subject is suffering from a disease, disorder or condition, e.g., a disease, disorder or condition that can be treated as provided herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Therapeutically effective amount: As used herein, is meant an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Threshold value: As used herein, the term “threshold value” refers to a value (or values) that are used as a reference to attain information on and/or classify the results of a measurement, for example, the results of a measurement attained in an assay. A threshold value can be determined based on one or more control samples. A threshold value can be determined prior to, concurrently with, or after the measurement of interest is taken. In some embodiments, a threshold value can be a range of values. In some embodiments, a threshold value can be a value (or range of values) reported in the relevant field (e.g., a value found in a standard table).

### DETAILED DESCRIPTION OF CERTAIN EMBODIMENTS

All percentages given herein refer to the weight percentages of a particular component relative to the entire composition, including the carrier, unless otherwise indicated. It will be understood that the sum of all weight % of individual components within a composition will not exceed 100%.

The terms “a” or “an,” as used in herein means one or more. As used herein, the term “consisting essentially of” is intended to limit the invention to the specified materials or steps and those that do not materially affect the basic and novel characteristics of the claimed invention, as understood from a reading of this specification. Recitations of “comprising” include “consisting essentially” and “consisting.”

In general, the limits (end points) of any range recited herein are within the scope of the invention and should be understood to be disclosed embodiments. Additionally, any half-integral value within that range is also contemplated. For example, a range of from 0 to 4 expressly discloses 0, 0.5, 1, 1.5, 2, 2.5, 3, 3.5, 4, and any subset within that range (e.g., from 1 to 2.5).

The term hydrocarbon may refer to a radical or group containing carbon and hydrogen atoms which may be bound at an indicated position (e.g., R, R′, R″, R^{N}, Y, Y′, Ω, L₁, L^{C}, R^{L}, R^{C}, R₁, R₂, R₂ₐ, R_{2b}, R_{2c}, R₃, R₄, R₅, R₆, R₇). Examples of hydrocarbon radicals include, without limitation, alkyl, alkenyl, alkynyl, aryl, aryl-alkyl, alkyl-aryl, and any combination thereof (e.g., alkyl-aryl-alkyl). As used herein, unless otherwise indicated, hydrocarbons may be monovalent or multivalent (e.g., divalent, trivalent) hydrocarbon radicals. A radical of the form —(CH₂)ₙ—, including a methylene radical, i.e., —CH₂—, is regarded as an alkyl radical if it does not have unsaturated bonds between carbon atoms. Unless otherwise specified, all hydrocarbon radicals (including substituted and unsubstituted alkyl, alkenyl, alkynyl, aryl, aryl-alkyl, alkyl-aryl) may have from 1-35 carbon atoms. In other embodiments, hydrocarbons will have from 1-20 or from 1-12 or from 1-8 or from 1-6 or from 1-3 carbon atoms, including for example, embodiments having one, two, three, four, five, six, seven, eight, nine, or ten carbon atoms. Hydrocarbons may have from 2 to 70 atoms or from 4 to 40 atoms or from 4 to 20 atoms.

A substituted hydrocarbon may have as a substituent one or more hydrocarbon radicals, substituted hydrocarbon radicals, or may comprise one or more heteroatoms. Any hydrocarbon substituents disclosed herein (e.g., R, R′, R″, R^{N}, Y, Y′, Ω, L₁, L^{C}, R^{L}, R^{C}, R₁, R₂, R₂ₐ, R_{2b}, R_{2c}, R₃, R₄, R₅, R₆, R₇) may optionally include from 1-20 (e.g., 1-10, 1-5) heteroatoms. Examples of substituted hydrocarbon radicals include, without limitation, heterocycles, such as heteroaryls. Unless otherwise specified, a hydrocarbon substituted with one or more heteroatoms will comprise from 1-20 heteroatoms. In other embodiments, a hydrocarbon substituted with one or more heteroatoms will comprise from 1-12 or from 1-8 or from 1-6 or from 1-4 or from 1-3 or from 1-2 heteroatoms. Examples of heteroatoms include, but are not limited to, oxygen, nitrogen, sulfur, phosphorous, halogen (e.g., F, Cl, Br, I), boron, or silicon. In some embodiments, heteroatoms will be selected from the group consisting of oxygen, nitrogen, sulfur, phosphorous, and halogen (e.g., F, Cl, Br, I). In certain embodiments, the heteroatoms may be selected from O, N, or S. In some embodiments, a heteroatom or group may substitute a carbon. In some embodiments, a heteroatom or group may substitute a hydrogen. In some embodiments, a substituted hydrocarbon may comprise one or more heteroatoms in the backbone or chain of the molecule (e.g., interposed between two carbon atoms, as in “oxa”). In some embodiments, a substituted hydrocarbon may comprise one or more heteroatoms pendant from the backbone or chain of the molecule (e.g., covalently bound to a carbon atom in the chain or backbone, as in “oxo”).

When an indicated group is substituted with an indicated substituent, the specified group may be substituted with one or more of any or all of the named substituents. For example, where a group, such as an alkyl or heteroaryl group, is substituted with an unsubstituted C₁-C₂₀ alkyl, or unsubstituted 2 to 20 membered heteroalkyl, the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different. If an indicated group is used multiple times in chemical genus (e.g., R groups), it will be understood that each group is independently selected at each occurrence.

Unless otherwise specified, any compound disclosed herein which has one or more chiral centers may be in the form of a racemic mixture with respect to each chiral center, or may exist as pure or substantially pure (e.g., great than 98% ee) R or S enantiomers with respect to each chiral center, or may exist as mixtures of R and S enantiomers with respect to each chiral center, wherein the mixture comprises an enantiomeric excess of one or the other configurations, for example an enantiomeric excess (of R or S) of more than 60% or more than 70% or more than 80% or more than 90%, or more than 95%, or more than 98%, or more than 99% enantiomeric excess. In some embodiments, any chiral center may be in the “S” or “R” configurations.

It will be understood that the description of compounds herein is limited by principles of chemical bonding. Accordingly, where a group may be substituted by one or more of a number of substituents, such substitutions are selected so as to comply with principles of chemical bonding such as regard to valencies, and to give compounds which are not inherently unstable. For example, any carbon atom will be bonded to two, three, or four other atoms, consistent with the four valence electrons of carbon.

Substituent (radical) prefix names may be derived from the parent hydride by either (i) replacing the “ane” or in the parent hydride with the suffixes “yl,” “diyl,” “triyl,” “tetrayl;” or (ii) replacing the “e” in the parent hydride with the suffixes “yl,” “diyl,” “triyl,” “tetrayl,” (here the atom(s) with the free valence, when specified, is (are) given numbers as low as is consistent with any established numbering of the parent hydride). Accepted contracted names, e.g., adamantyl, naphthyl, anthryl, phenanthryl, furyl, pyridyl, isoquinolyl, quinolyl, and piperidyl, and trivial names, e.g., vinyl, allyl, phenyl, and thienyl are also used herein throughout.

Alkyl groups typically refer to a saturated hydrocarbon chain that may be a straight chain or branched chain, containing the indicated number of carbon atoms. For example, C₁-C₆ alkyl indicates that the group may have from 1 to 6 (inclusive) carbon atoms in it. Any atom can be optionally substituted, e.g., by one or more substituents. Examples of alkyl groups include without limitation methyl, ethyl, n-propyl, isopropyl, and tert-butyl. Any alkyl group referenced herein (e.g., R, R′, R″, R^{N}, Y, Y′, Ω, L₁, L^{C}, R^{L}, R^{C}, R₁, R₂, R₂ₐ, R_{2b}, R_{2c}, R₃, R₄, R₅, R₆, R₇) may have from 1-35 carbon atoms. In other embodiments, alkyl groups will have from 1-20 or from 1-12 or from 1-8 or from 1-6 or from 1-3 carbon atoms, including for example, embodiments having one, two, three, four, five, six, seven, eight, nine, or ten carbon atoms. Alkyl groups may be lower alkyl (e.g., C₁-C₄ alkyl).

Haloalkyl groups are typically alkyl groups where at least one hydrogen atom is replaced by halo. In some embodiments, more than one hydrogen atom (e.g., 2, 3, 4, 5, 6, 7, 8, 9, 10, 11, 12, 13, or 14) are replaced by halo. In these embodiments, the hydrogen atoms can each be replaced by the same halogen (e.g., fluoro) or the hydrogen atoms can be replaced by a combination of different halogens (e.g., fluoro and chloro). Haloalkyl may include alkyl moieties in which all hydrogens have been replaced by halo (sometimes referred to herein as perhaloalkyl, e.g., perfluoroalkyl, such as trifluoromethyl). Haloalkyl groups may be optionally substituted.

Typically, alkoxy groups have the formula —O(alkyl). Alkoxy can be, for example, methoxy (—OCH₃), ethoxy, propoxy, isopropoxy, butoxy, iso-butoxy, sec-butoxy, pentoxy, 2-pentoxy, 3-pentoxy, or hexyloxy. Likewise, the term “thioalkoxy” refers to a group of formula —S(alkyl). Finally, the terms “haloalkoxy” and “halothioalkoxy” refer to —O(haloalkyl) and —S(haloalkyl), respectively. The term “sulfhydryl” refers to —SH. As used herein, the term “hydroxyl,” employed alone or in combination with other terms, refers to a group of formula —OH. Any alkoxy, thioalkoxy, or haloalkoxy group referenced herein (e.g., R, R′, R″, R^{N}, Y, Y′, Ω, L₁, L^{C}, R^{L}, R^{C}, R₁, R₂, R₂ₐ, R_{2b}, R_{2c}, R₃, R₄, R₅, R₆, R₇) may have from 1-35 carbon atoms. In other embodiments, alkoxy, thioalkoxy, or haloalkoxy groups will have from 1-20 or from 1-12 or from 1-8 or from 1-6 or from 1-3 carbon atoms, including for example, embodiments having one, two, three, four, five, six, seven, eight, nine, or ten carbon atoms. Alkoxy groups may be lower alkoxy (e.g., C₁-C₄ alkoxy).

Aralkyl groups typically refers to groups where an alkyl moiety in which an alkyl hydrogen atom is replaced by an aryl group. One of the carbons of the alkyl moiety serves as the point of attachment of the aralkyl group to another moiety. Any ring or chain atom can be optionally substituted, e.g., by one or more substituents. Non-limiting examples of aralkyl include benzyl, 2-phenylethyl, and 3-phenylpropyl groups.

The term alkenyl may refer to a straight or branched hydrocarbon chain containing the indicated number of carbon atoms and having one or more carbon-carbon double bonds. Any atom can be optionally substituted, e.g., by one or more substituents. Alkenyl groups can include, e.g., vinyl, allyl, 1-butenyl, and 2-hexenyl. One of the double bond carbons can optionally be the point of attachment of the alkenyl substituent. Any alkenyl group referenced herein (e.g., R, R′, R″, R^{N}, Y, Y′, Ω, L₁, L^{C}, R^{L}, R^{C}, R₁, R₂, R₂ₐ, R_{2b}, R_{2c}, R₃, R₄, R₅, R₆, R₇) may have from 1-35 carbon atoms. In other embodiments, alkenyl groups will have from 1-20 or from 1-12 or from 1-8 or from 1-6 or from 1-3 carbon atoms, including for example, embodiments having one, two, three, four, five, six, seven, eight, nine, or ten carbon atoms.

The term alkynyl may refer to a straight or branched hydrocarbon chain containing the indicated number of carbon atoms and having one or more carbon-carbon triple bonds. Alkynyl groups (e.g., R, R′, R″, R^{N}, Y, Y′, Ω, L₁, L^{C}, R^{L}, R^{C}, R₁, R₂, R₂ₐ, R_{2b}, R_{2c}, R₃, R₄, R₅, R₆, R₇) can be optionally substituted, e.g., by one or more substituents. Alkynyl groups can include, e.g., ethynyl, propargyl, and 3-hexynyl. One of the triple bond carbons can optionally be the point of attachment of the alkynyl substituent.

The term heterocyclyl typically refers to a fully saturated, partially saturated, or aromatic monocyclic, bicyclic, tricyclic, or other polycyclic ring system having one or more constituent heteroatom ring atoms independently selected from O, N (it is understood that one or two additional groups (e.g., R^{N}) may be present to complete the nitrogen valence and/or form a salt), or S. The heteroatom or ring carbon can be the point of attachment of the heterocyclyl substituent to another moiety. Any atom can be optionally substituted, e.g., with one or more substituents (e.g. heteroatoms or substituent groups X). Heterocyclyl groups can include, e.g., tetrahydrofuryl, tetrahydropyranyl, piperidyl (piperidino), piperazinyl, morpholinyl (morpholino), pyrrolinyl, and pyrrolidinyl. By way of example, the phrase “heterocyclic ring containing from 5-6 ring atoms, wherein from 1-2 of the ring atoms is independently selected from N, NH, N(C₁-C₆ alkyl), NC(O)(C₁-C₆ alkyl), O, and S; and wherein said heterocyclic ring is optionally substituted with from 1-3 independently selected R″ would include (but not be limited to) tetrahydrofuryl, tetrahydropyranyl, piperidyl (piperidino), piperazinyl, morpholinyl (morpholino), pyrrolinyl, and pyrrolidinyl.

The term heterocycloalkenyl typically refers to partially unsaturated monocyclic, bicyclic, tricyclic, or other polycyclic hydrocarbon groups having one or more (e.g., 1-4) heteroatom ring atoms independently selected from O, N (it is understood that one or two additional groups may be present to complete the nitrogen valence and/or form a salt), or S. A ring carbon (e.g., saturated or unsaturated) or heteroatom can be the point of attachment of the heterocycloalkenyl substituent. Any atom can be optionally substituted, e.g., by one or more substituents. Heterocycloalkenyl groups can include, e.g., dihydropyridyl, tetrahydropyridyl, dihydropyranyl, 4,5-dihydrooxazolyl, 4,5-dihydro-1H-imidazolyl, 1,2,5,6-tetrahydro-pyrimidinyl, and 5,6-dihydro-2H-[1,3]oxazinyl.

Cycloalkyl groups may be fully saturated monocyclic, bicyclic, tricyclic, or other polycyclic hydrocarbon groups. Any atom can be optionally substituted, e.g., by one or more substituents. A ring carbon serves as the point of attachment of a cycloalkyl group to another moiety. Cycloalkyl moieties can include, e.g., cyclopropyl, cyclobutyl, cyclopentyl, cyclohexyl, cycloheptyl, adamantyl, and norbornyl (bicyclo[2.2.1]heptyl).

Cycloalkenyl groups may be partially unsaturated monocyclic, bicyclic, tricyclic, or other polycyclic hydrocarbon groups. A ring carbon (e.g., saturated or unsaturated) is the point of attachment of the cycloalkenyl substituent. Any atom can be optionally substituted, e.g., by one or more substituents. Cycloalkenyl moieties can include, e.g., cyclohexenyl, cyclohexadienyl, or norbornenyl.

Aryl groups are often aromatic monocyclic, bicyclic (2 fused rings), tricyclic (3 fused rings), or polycyclic (>3 fused rings) hydrocarbon ring system. One or more ring atoms can be optionally substituted, e.g., by one or more substituents. Aryl moieties include, e.g., phenyl and naphthyl.

Heteroaryl groups typically are aromatic monocyclic, bicyclic (2 fused rings), tricyclic (3 fused rings), or polycyclic (>3 fused rings) hydrocarbon groups having one or more heteroatom ring atoms independently selected from O, N (it is understood that one or two additional groups may be present to complete the nitrogen valence and/or form a salt), or S in the ring. One or more ring atoms can be optionally substituted, e.g., by one or more substituents. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, coumarinyl, chromenyl, cinnolinyl, dibenzo[b,d]furanyl, furazanyl, furyl, imidazolyl, imidizolyl, indazolyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, and xanthenyl.

In general, when a definition for a particular variable includes both hydrogen and non-hydrogen (halo, alkyl, aryl) possibilities, the term “substituent(s) other than hydrogen” refers collectively to the non-hydrogen possibilities for that particular variable, unless otherwise specified.

The term “substituent” may refer to a group “substituted” on, on a hydrocarbon (e.g., an alkyl, haloalkyl, cycloalkyl, heterocyclyl, heterocycloalkenyl, cycloalkenyl, aryl, heteroaryl) group at any atom of that group, typically replacing one or more hydrogen atoms therein. In one aspect, the substituent(s) on a group (e.g., R, R′, R″, R^{N}, Y, Y′, Ω, L₁, L^{C}, R^{L}, R^{C}, R₁, R₂, R₂ₐ, R_{2b}, R_{2c}, R₃, R₄, R₅, R₆, R₇) are independently any one single, or any combination of two or more of the permissible atoms or groups of atoms delineated for that substituent. In another aspect, a substituent may itself be substituted with any one of the above substituents. In some embodiments, an indicated substituent is not further substituted. Further, as used herein, the phrase “optionally substituted” means unsubstituted (e.g., substituted with an H) or substituted. It is understood that substitution at a given atom is limited by valency. Common substituents include halo (e.g. F), C₁₋₁₂ straight chain or branched chain alkyl, C₂₋₁₂ alkenyl, C₂₋₁₂ alkynyl, C₃₋₁₂ cycloalkyl, C₆₋₁₂ aryl, C₃₋₁₂ heteroaryl, C₃₋₁₂ heterocyclyl, C₁₋₁₂ alkylsulfonyl, nitro, cyano, —COOR, —C(O)NRR′, —OR, —SR, —NRR′, and oxo, such as mono- or di- or tri-substitutions with moieties such as trifluoromethoxy, chlorine, bromine, fluorine, methyl, methoxy, pyridyl, furyl, triazyl, piperazinyl, pyrazoyl, imidazoyl, and the like, each optionally containing one or more heteroatoms such as halo, N, O, S, and P. R and R′ are independently hydrogen, C₁₋₁₂ alkyl, C₁₋₁₂ haloalkyl, C₂₋₁₂ alkenyl, C₂₋₁₂ alkynyl, C₃₋₁₂ cycloalkyl, C₄₋₂₄ cycloalkylalkyl, C₆₋₁₂ aryl, C₇₋₂₄ aralkyl, C₃₋₁₂ heterocyclyl, C₃₋₂₄ heterocyclylalkyl, C₃₋₁₂ heteroaryl, or C₄₋₂₄ heteroarylalkyl. Unless otherwise noted, all groups described herein optionally contain one or more common substituents, to the extent permitted by valency. The term “substituted” typically means that a hydrogen and/or carbon atom is removed and replaced by a substituent (e.g., a common substituent). The use of a substituent (radical) prefix names such as alkyl without the modifier “optionally substituted” or “substituted” is understood to mean that the particular substituent is unsubstituted. However, the use of “haloalkyl” without the modifier “optionally substituted” or “substituted” is still understood to mean an alkyl group, in which at least one hydrogen atom is replaced by halo and any other associated substitutions as necessary. Any hydrocarbon described herein may be considered optionally substituted.

The present disclosure involves conjugates useful in high sensitive immunoassays for the detection of an analyte. Typically, these conjugates have a chemiluminescent acridinium conjugated to a carrier protein, wherein the carrier protein is conjugated to a antibody or antibody fragment (e.g., F(ab)) via a linker (e.g., PEG such as PEG₂-PEG₂₀ or PEG₃-PEG₁₀). In certain embodiments the conjugate comprises an excess of acridinium moieties (e.g., from 2-40, from 5-25) in relation to the carrier protein. In some embodiments, the first reacting step occurs in a medium comprising a buffer. In various implementations, the chemiluminescent acridinium compound is added in weight excess of the carrier protein (e.g., less than 100× excess or less than 50× excess or less than 40× excess or less than 30× excess or from 5× excess to 25× excess).

Methods disclosed herein may be used to, for example, identify, diagnose, monitor, or treat neurological diseases or conditions. Embodiments described herein provide a number of advantages over the prior techniques discussed herein. For example, technologies of the present disclosure are non-invasive, require minimal patient discomfort, are quick to perform, and are relatively cost-effective. Accordingly, technologies described herein offer advantages over these prior techniques including, but not limited to providing: a non-invasive in vitro diagnostic (IVD) test for neurological disease or condition, or to rule out neurological disease or condition.

The present disclosure relates particularly to NfL as a biomarker for neurological disease, and an assay for detection of the biomarker. In some embodiments, NfL as described herein is or includes NfL protein, nucleic acid sequences encoding NfL, characteristic fragments thereof, and/or variants thereof.

As provided herein, NfL includes gene products associated with NfL. For example, NfL can include, for example, a protein or nucleotide (e.g., RNA, e.g., mRNA). NfL also encompasses full-length proteins, as well as fragments (e.g., characteristic fragments) of NfL. In some embodiments, NfL includes a fragment having an amino acid sequence identical to a contiguous span of at least 10 amino acids, at least 20 amino acids, at least 30 amino acids, at least 40 amino acids, at least 50 amino acids, at least 60 amino acids, at least 70 amino acids, at least 80 amino acids, at least 90 amino acids, or at least 100 amino acids of an amino acid sequence provided in Table 1. In some embodiments, NfL includes a fragment having at least 70%, at least 75%, at least 80%, at least 85%, at least 90%, at least 95%, at least 96%, at least 97%, at least 98%, or at least 99% identity to an amino acid sequence provided in Table 1. In some embodiments, NfL includes a nucleic acid fragment having a nucleic acid sequence identical to a contiguous span of at least 10 nucleic acids, at least 20 nucleic acids, at least 30 nucleic acids, at least 40 nucleic acids, at least 50 nucleic acids, at least 60 nucleic acids, at least 70 nucleic acids, at least 80 nucleic acids, at least 90 nucleic acids, or at least 100 nucleic acids of a nucleic acid sequence provided in Table 2. In some embodiments, NfL includes a fragment having at least 70%, at least 75%, at least 80%, at least 85%, at least 90%, at least 95%, at least 96%, at least 97%, at least 98%, or at least 99% identity to a nucleic acid sequence provided in Table 2. Variant or alternative forms of NfL include for example polypeptides encoded by any splice-variants of transcripts encoding NfL.

Biomarkers contemplated herein also include truncated forms or polypeptide fragments of NfL as described herein. Truncated forms or polypeptide fragments of NfL can include N-terminally deleted or truncated forms and C-terminally deleted or truncated forms. Truncated forms or fragments of NfL can include fragments arising by any mechanism, such as, without limitation, by alternative translation, exo- and/or endo-proteolysis and/or degradation, for example, by physical, chemical and/or enzymatic proteolysis. Without limitation, a biomarker may include a truncated or fragment of NfL may include at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 8%, or at least 99% of the amino acid sequence of an NfL protein.

In some instances, a fragment is N-terminally and/or C-terminally truncated by 1-20 amino acids, such as, for example, by 1-15 amino acids, by 1-10 amino acids, or by 1-5 amino acids, compared to the corresponding mature, full-length NfL protein.

NfL protein of the present disclosure such as an NfL protein or fragments thereof may also encompass modified forms of NfL such as bearing post-expression modifications including but not limited to, modifications such as phosphorylation, glycosylation, lipidation, methylation, selenocystine modification, cysteinylation, sulphonation, glutathionylation, acetylation, and/or oxidation of methionine to methionine sulphoxide or methionine sulphone.

In some embodiments, NfL can be a nucleotide (also referred to herein as a nucleic acid or polynucleotide). In some embodiments, a nucleotide can be RNA or DNA (e.g., cDNA). In some cases, corresponding RNA or DNA may exhibit better discriminatory power in diagnosis than the full-length protein.

A brief overview of NfL is provided below.

### Neurofilament Light Chain (NfL)

Neurofilaments are cytoskeletal components of neurons that are particularly abundant in axons. The functions of neurofilaments include provision of structural support and maintenance of the size, shape, and caliber of axons. Neurofilaments include three subunits: neurofilament light chain (NfL), neurofilament medium chain, and neurofilament heavy chain. NfL levels increase in cerebrospinal fluid (CSF) and blood proportionally to the degree of axonal damage in a variety of neurological disorders, including inflammatory, neurodegenerative, traumatic and cerebrovascular diseases. While NfL has been used as a biomarker for neurodegenerative disorders, its connection to other diseases and conditions, including CNS conditions, has not been fully explored.

An exemplary amino acid sequence for NfL is included in Table 1 below.

TABLE 1
Exemplary NfL Amino Acid Sequence

| Biomarker | UniProt ID | Exemplary Amino Acid Sequence |
|---|---|---|
| NfL | P07196 | MSSFSYEPYYSTSYKRRYVETPRVHISSVRSGYST |
| | | ARSAYSSYSAPVSSSLSVRRSYSSSSGSLMPSLEN |
| | | LDLSQVAAISNDLKSIRTQEKAQLQDLNDRFASFIER |
| | | VHELEQQNKVLEAELLVLRQKHSEPSRFRALYEQEI |
| | | RDLRLAAEDATNEKQALQGEREGLEETLRNLQARY |
| | | EEEVLSREDAEGRLMEARKGADEAALARAELEKRI |
| | | DSLMDEISFLKKVHEEEIAELQAQIQYAQISVEMDVT |
| | | KPDLSAALKDIRAQYEKLAAKNMQNAEEWFKSRFT |
| | | VLTESAAKNTDAVRAAKDEVSESRRLLKAKTLEIEA |
| | | CRGMNEALEKQLQELEDKQNADISAMQDTINKLEN |
| | | ELRTTKSEMARYLKEYQDLLNVKMALDIEIAAYRKLL |
| | | EGEETRLSFTSVGSITSGYSQSSQVFGRSAYGGLQ |
| | | TSSYLMSTRSFPSYYTSHVQEEQIEVEETIEAAKAE |
| | | EAKDEPPSEGEAEEEEKDKEEAEEEEAAEEEEAAK |
| | | EESEEAKEEEEGGEGEEGEETKEAEEEEKKVEGA |
| | | GEEQAAKKKD (SEQ ID NO: 1) |

An exemplary nucleic acid sequence for NfL is included in Table 2 below.

TABLE 2
Exemplary NfL Nucleic Acid Sequence

| Biomarker | Exemplary Nucleic Acid Sequence |
|---|---|
| NfL | gcacacagcc atccatcctc ccccttccct ctctcccctg tcctctctct |
| | ccgggctccc accgccgccg cgggccgggg agccaccggc cgccaccatg |
| | agttccttca gctacgagcc gtactactcg acctcctaca agcggcgcta |
| | cgtggagacg ccccgggtgc acatctccag cgtgcgcagc ggctacagca |
| | ccgcacgctc agcttactcc agctactcgg cgccggtgtc ttcctcgctg |
| | tccgtgcgcc gcagctactc ctccagctct ggatcgttga tgcccagtct |
| | ggagaacctc gacctgagcc aggtagccgc catcagcaac gacctcaagt |
| | ccatccgcac gcaggagaag gcgcagctcc aggacctcaa tgaccgcttc |
| | gccagcttca tcgagcgcgt gcacgagctg gagcagcaga acaaggtcct |
| | ggaagccgag ctgctggtgc tgcgccagaa gcactccgag ccatcccgct |
| | tccgggcgct gtacgagcag gagatccgcg acctgcgcct ggcggcggaa |
| | gatgccacca acgagaagca ggcgctccag ggcgagcgcg |
| | aagggctgga ggagaccctg cgcaacctgc aggcgcgcta tgaagaggag |
| | gtgctgagcc gcgaggacgc cgagggccgg ctgatggaag cgcgcaaagg |
| | cgccgacgag gcggcgctcg ctcgcgccga gctcgagaag cgcatcgaca |
| | gcttgatgga cgaaatctct tttctgaaga aagtgcacga agaggagatc |
| | gccgaactgc aggcgcagat ccagtacgcg cagatctccg tggagatgga |
| | cgtgaccaag cccgaccttt ccgccgcgct caaggacatc cgcgcgcagt |
| | acgagaagct ggccgccaag aacatgcaga acgctgagga atggttcaag |
| | agccgcttca ccgtgctgac cgagagcgcc gccaagaaca ccgacgccgt |
| | gcgcgccgcc aaggacgagg tgtccgagag ccgtcgtctg ctcaaggcca |
| | agaccctgga aatcgaagca tgccggggca tgaatgaagc gctggagaag |
| | cagctgcagg agctggagga caagcagaac gccgacatca gcgctatgca |
| | ggacacgatc aacaaattag aaaatgaatt gaggaccaca aagagtgaaa |
| | tggcacgata cctaaaagaa taccaagacc tcctcaacgt gaagatggct |
| | ttggatattg agattgcagc ttacaggaaa ctcttggaag gcgaggagac |
| | ccgactcagt ttcaccagcg tgggaagcat aaccagtggc tactcccaga |
| | gctcccaggt ctttggccga tctgcctacg gcggtttaca gaccagctcc |
| | tatctgatgt ccacccgctc cttcccgtcc tactacacca gccatgtcca |
| | agaggagcag atcgaagtgg aggaaaccat tgaggctgcc aaggctgagg |
| | aagccaagga tgagcccccc tctgaaggag aagccgagga |
| | ggaggagaag gacaaggaag aggccgagga agaggaggca |
| | gctgaagagg aagaagctgc caaggaagag tctgaagaag |
| | caaaagaaga agaagaagga ggtgaaggtg aagaaggaga |
| | ggaaaccaaa gaagctgaag aggaggagaa gaaagttgaa |
| | ggtgctgggg aggaacaagc agctaagaag aaagattgaa cccccatttc |
| | cttaattatt tcaggaataa ttctcccgaa atcaggtcaa ccccatcacc |
| | aaccaaccaa ccagttgagt tccagattct atgtgaatta aaaagtcaat |
| | atatgtataa ttctgagatg acttaggttg gacattcaat gttgtgctat |
| | gaatttcctc tttatgcaga gtatctgttt gcttgcagag tggctttctg |
| | gcttgctgcc agcctgtgca tggtccacgc ttatgagttc aggatctacg |
| | gcaatgtgaa tcattcagat gtttacaata aaaaacacca catgagtaaa |
| | tgaattcact aatgttaatg ttaaacttca tggaaaaata gtcctttgaa |
| | ccttcggtgg ttagcaatta aagaccctga gttatgtgca ataaatagta |
| | aataaagtta taccgaatga tgtatttttt gctgtggttg ttacttaatt aaaatacctt |
| | aaagatggca ccaatataaa gtatatacca gtggactatt gacctccaat |
| | tttttaaaaa gttgaaattt taacaattac caatactttt ttttcttcct tcaattggaa |
| | attctgaggg atacagttat gtcatgatta cttgtgaaat ctctccccac |
| | aaaaatttta ctgataataa acatgagtaa atgagaaagt ctataaataa |
| | caatacactt tgcctcataa cacgtgttgt gttaggtttt gggatggctt |
| | tctagggagc aattctgaaa ttacttgaca gatacactct ttatggtaaa |
| | caggtcattt cagagtctag agtgctaacc attacactat ggaaccatga |
| | taaataggtc atttcaaact gatatgtcac aatgcaatat ggatagaata |
| | tgaatcccat acattcatcc ctataactgc attttaaaat atagtctaaa |
| | tatcatggga ggaggttaaa acctgagaac atggtacaat aagaaggaat |
| | aattttttat ctatatataa tttcaggaga agggattaaa actaccaaag |
| | gttatttgcc actccactgc aaacatgttg cctgcaatcc tgagctcatg |
| | atttagtgtg ttgttaagcg taattctgtt gcctcaagta aataccactt |
| | aatgctacac atttttaaac ctttaaaagc tacagacaca agtaatgaaa |
| | ttatgagtca cttaaggggg gaggtaatat gcttgtatgt tacattgaca |
| | ttgctctgca tgtctctact gggaaatatt tggatttcaa tgatagcttg |
| | actggttgga cattgcatca aatacacgcc ccatgtatcc tttctgtcgt |
| | aaagttaaga acgctcttgc atttgatgtg gaataaatat agatgatatt |
| | acaaaaaaaa aaaaaaaaaa aaaaaaaaaa aa (SEQ ID NO: 2) |

As provided herein, NfL may be a full length protein or a fragment thereof, or functional fragment thereof. In some embodiments, a fragment of NfL is a characteristic protein fragment. In some embodiments, NfL is a full-length protein. In some embodiments, NfL, e.g., in a sample, can include a subset of full-length NfL proteins and a subset of characteristic protein fragments of NfL.

In some embodiments, NfL has a wild-type amino acid sequence. In some embodiments, NfL has a variant amino acid sequence, e.g., an amino acid sequence including one or more mutations. In some embodiments, a subset of NfL proteins have a wild-type amino acid sequence and a subset of NfL proteins have a variant amino acid sequence.

As provided herein, NfL may be a full-length nucleotide (e.g., DNA, cDNA, or RNA) that encodes NfL or a fragment thereof. In some embodiments, a fragment of NfL is a characteristic nucleotide fragment. In some embodiments, NfL is a full length nucleotide (e.g., DNA, cDNA, or RNA) that encodes NfL. In some embodiments, NfL, e.g., in a sample, can include a subset of full length NfL nucleotides (e.g., DNA, cDNA, or RNA) and a subset of characteristic nucleotide fragments of NfL.

In some embodiments, NfL has a wild-type nucleic acid sequence. In some embodiments, NfL has a variant nucleic acid sequence, e.g., a nucleic acid sequence including one or more mutations. In some embodiments, a subset of NfL includes a wild-type nucleic acid sequence that encodes NfL and a subset includes a variant nucleic acid sequence that encodes NfL.

### Methods

Methods of the present disclosure further allow for earlier identification of more patients who are at risk of neurological disease, while minimizing the number of false negatives. In some embodiments, methods disclosed herein provide the advantage of an early screen for the presence of neurological disease. In some embodiments, methods disclosed herein can assist in the detection or diagnosis of neurological disease, e.g., after genetic testing rules out neurological disease. In some embodiments, methods disclosed herein reduces or eliminates the need for initiating screening for neurological disease. In some embodiments, if methods disclosed herein result in detection of possible neurological disease in a subject, the subject can undergo subsequent confirmatory testing, such as biopsy.

In some embodiments, a method disclosed herein is a method of determining a subject's risk of developing neurological disease. In some embodiments, a method disclosed herein is a method of diagnosing a subject with neurological disease, and the sample was obtained from the subject. In some embodiments, a method disclosed herein is a method of treating neurological disease in a subject at risk of or suffering from neurological disease. In some embodiments, a method disclosed herein is a method of determining a patient does not have or is not at risk of developing neurological disease.

In some embodiments, a method disclosed herein is a method of selecting a subject to receive one or more doses of a medication, and the sample was obtained from the subject. In some embodiments, a method disclosed herein includes administering to the subject one or more doses a medication.

The present disclosure provides diagnostic tests for neurological disease characterized by detection of NfL according to the methods above.

In some embodiments, methods of detecting, diagnosing, or identifying a risk of neurological disease as taught by the present disclosure are improved methods as compared to standard techniques in that the methods of the present disclosure include one or more of the following benefits: improved sensitivity for identifying neurological disease, improved specificity for identifying neurological disease, improved accuracy for identifying neurological disease, reduced time to diagnosis for neurological disease, and/or reduced cost of screening patients for neurological disease.

In some embodiments, NfL can be used for an in-vitro diagnostic (IVD) or screening test for the condition of neurological disease. In some embodiments, a diagnostic test as taught by the present disclosure detects whether NfL is present in a sample obtained from a subject. In some embodiments, a diagnostic test as taught by the present disclosure can assist in the detection or diagnosis of neurological disease in a subject.

In some embodiments, a diagnostic test as taught by the present disclosure is adapted to an immunoassay platform. In some embodiments, such an immunoassay platform includes a semi-automated or automated immunoassay platform. In some embodiments, a diagnostic test as taught by the present disclosure is adapted for semi-automated testing of one or more biomarkers. In some embodiments, a diagnostic test as disclosed herein can be a plasma-based screening assay. In some embodiments, a diagnostic test is adapted for, e.g., the Siemens Atellica® system or the Siemens Advia Centaur® system.

As set out in greater detail below, in some embodiments, methods provided herein include detecting a level of NfL present in a sample.

In some embodiments, methods provided herein include detecting a level of NfL in a sample to obtain a biomarker profile, and using the biomarker profile to compute an biomarker score. In some embodiments, methods provided herein include detecting a level of NfL in a sample to obtain a biomarker profile, and using the biomarker profile and demographic factors to compute a biomarker score. In some embodiments, methods provided herein include detecting a level of NfL in a sample to obtain a biomarker profile, and using the biomarker profile and imaging-based biomarkers to compute a biomarker score. In some embodiments, methods provided herein include detecting a level of NfL in a sample to obtain a biomarker profile, and using the biomarker profile, demographic factors, and imaging-based biomarkers to compute a biomarker score.

In some embodiments, methods provided herein including receiving a level of NfL, demographic factors and/or imaging-based biomarkers in a sample. In some embodiments, receiving includes electronically receiving.

### Detecting NfL and Obtaining a Biomarker Profile

Among other things, methods provided herein include assessment of a level of NfL in a sample. A level of NfL can be detected in a sample. Exemplary methods for detecting a level of NfL are described herein. However, a level of NfL can also be provided, for example, in electronic form from, e.g., a laboratory that has detected a level of NfL in sample.

In some embodiments, the present disclosure provides technologies according to which NfL is detected, analyzed and/or assessed in a sample. In some embodiments, NfL is in a sample obtained from a subject; in some embodiments, a diagnosis or therapeutic decision is made based on such detection, analysis and/or assessment.

In some embodiments, a level of NfL, as described herein, encompasses the presence of NfL, the absence of NfL, an amount of NfL, an absolute amount of NfL, a relative amount of NfL, or a concentration of NfL.

Methods of detecting NfL include methods for detecting biomarkers as proteins. Protein-based methods of detecting biomarkers include, for example, mass spectrometry (MS), immunoassays (e.g., immunoprecipitation), Western blots, ELISAs, immunohistochemistry, immunocytochemistry, flow cytometry, and/or immuno-PCR.

In some embodiments, mass spectrometry includes MS, MS/MS, MALDI-TOF, electrospray ionization mass spectrometry (ESIMS), ESI-MS/MS, ESI-MS/(MS)ₙ, matrix-assisted laser desorption ionization time-of-flight mass spectrometry (MALDI-TOF-MS), surface-enhanced laser desorption/ionization time-of-flight mass spectrometry (SELDI-TOF-MS), tandem liquid chromatography-mass spectrometry (LC-MS/MS) mass spectrometry, desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS), atmospheric pressure photoionization mass spectrometry (APPI-MS), APPI-MS/MS, and APPI-(MS)ₙ, quadrupole mass spectrometry, Fourier transform mass spectrometry (FTMS), and ion trap mass spectrometry. Often, the MS approach quantifies a fragment of a biomarker rather than the full-length protein. MS approaches, however, can be sufficient to determine the protein level of the biomarker to an accuracy sufficient for methods and/or assessments as disclosed.

In some embodiments, an immunoassay can be a chemiluminescent immunoassay. In some embodiments, an immunoassay can be a high-throughput and/or automated immunoassay platform. For example, a high-throughput and/or automated immunoassay platform can be used to analyze at least 240 tests per hour or at least 440 tests per hour.

In some embodiments, methods of detecting NfL in a sample includes contacting a sample with one or more antibody agents directed to NfL. In some embodiments, such methods also include contacting the sample with a first set of one or more detection agents. In some embodiments, the antibody agents are labeled with the first set of one or more detection agents. In some embodiments, the first set of one or more detection agents include one or more acridinium ester molecules.

Acridinium ester (AE) molecules, such as those described herein, can be used to label proteins and nucleic acids. Acridinium-labeled proteins can be used for detection in immunoassays. Exposing AE to an alkaline H₂O₂ (hydrogen peroxide) produces chemiluminscence. Light is emitted at a wavelength maximum in the range of 430 to 480 nm, depending on the specific AE variant. Such light can be detected, for example, by high-efficiency photomultiplier tubes. The light emission is rapid and completes within 1 to 5 seconds. Diversity in AE forms contributes to better assay performance, including improved sensitivity and robustness. AE molecules can be used to label small molecules, large analytes, and antibodies.

Chemiluminescence from acridinium labels can be measured by using photomultiplier tubes (PMT), each of the PMT's which may be equipped with an optical filter that allows the light from an acridinium ester of interest to pass through while blocking the unwanted light. Yet another alternative detector is a charge-coupled device (CCD). The chemiluminescence may pass through a grating, such that the wavelength separation may occur along the detector (e.g., CCD detector) and the images can be analyzed accordingly.

The acridinium labels of U.S. Pat. No. 8,119,422, which is hereby incorporated by reference in its entirety, can be used as materials to form acridinium labels.

Additional methods of detecting biomarkers (e.g., NfL) include methods for detecting biomarkers as nucleic acids. Nucleic acid-based methods of detecting NfL include performing nucleic acid amplification methods, such as polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). In some embodiments, a nucleic acid-based method of detecting biomarkers includes detecting hybridization between one or more nucleic acid probes and one or more nucleotides that encode NfL. In some embodiments, the nucleic acid probes are each complementary to at least a portion of one of the one or more nucleotides that encode NfL. In some embodiments, the nucleotides that encode NfL include DNA (e.g., cDNA). In some embodiments, the nucleotides that encode NfL include RNA (e.g., mRNA).

A variety of immunoassay formats, including, for example, competitive and non-competitive immunoassay formats, antigen/analyte capture assays, and two-antibody sandwich assays can be used in accordance with the cartridges, kits, and methods described herein. The assay may be, for example, a competitive immunoassay which typically involves the detection of a large molecule, also referred to as macromolecular analyte, using binding molecules such as antibodies. The antibody is immobilized or attached to a solid phase such as a particle, bead, membrane, microtiter plate, or any other solid surface.

In an example of a competitive heterogeneous assay, a support having an antibody for an analyte (e.g., bovine monoclonal antibodies, mouse monoclonal antibodies, antibody fragments such as bovine antibody fragments, mouse antibody fragments) bound thereto is contacted with a medium containing a sample suspected of containing the analyte and the chemiluminescent conjugates (or “labeled analogs”) described herein. Analyte from the sample may compete for binding to the analyte antibody with the labeled analog. After separating the support and the medium, the label activity of the support or the medium is determined by conventional techniques and is related to the amount of analyte in the sample. In a variation of the above competitive heterogeneous assay, the support comprises the analyte analog, which competes with analyte of the sample for binding to an antibody reagent in accordance with the principles described herein. The labeled analyte analog may be covalently attached with a chemiluminescent or fluorescent molecule often referred to as a label or tracer.

When the solid phase with the immobilized antibody is mixed with a sample containing the analyte and the labeled analyte, a binding complex is typically formed between the analyte or the labeled analyte. This type of assay is often called a heterogeneous assay because of the involvement of a solid phase. The chemiluminescent signal associated with the binding complex can then be measured and the presence or absence of the analyte in the sample can be inferred. Usually, the binding complex is separated from the rest of the binding reaction components such as excess, labeled analyte, prior to signal generation. For example, if the binding complex is associated with a magnetic bead, a magnet can be used to separate the binding complex associated with the bead from bulk solution.

In an example of a sandwich assay format employing two antibodies (or fragments thereof), a solid support with a first immobilized antibody or fragment thereof for an analyte is mixed with a sample containing the analyte and a labelled conjugate comprising a second antibody or fragment thereof. A binding complex is formed between the solid particle and the labelled conjugate via the analyte in the sample. The signal associated with the binding complex and can the measured and the presence or absence or amount of analyte can be inferred. Usually, the binding complex is separated from the rest of the binding reaction components such as excess, labeled analyte, prior to signal generation. For example, if the binding complex is associated with a magnetic bead, a magnet can be used to separate the binding complex associated with the bead from bulk solution. In some embodiments, the first immobilized antibody is a biotinylated mouse monoclonal antibody bound to coated (e.g., streptavidin coated) optionally paramagnetic particles. In some embodiments, the second antibody is a mouse monoclonal antibody fragment labelled with acridinium (e.g., acridinium ester).

By using a series of “standards,” that is, known concentrations of the analyte, a “dose-response” curve can be generated for the known labeled analyte. These dose response curves may be identified individually for any acridinium label or identified based on combinations of the acridinium labels used in the assay. Thus, the dose-response curve correlates a certain amount of measured signal with a specific concentration of analyte. In a competitive assay, as the concentration of the analyte increases, the amount of signal decreases if the chemiluminescence from the binding complex is measured. The concentration of the analyte in an unknown sample can then be calculated by comparing the signal generated by an unknown sample containing the macromolecular analyte, with the dose-response curve.

The methodology of the attachment of binding molecules such as antibodies to solid phases typically involves a mixing of the requisite components to induce attachment. For example, an antibody can be covalently attached to a particle containing amines on its surface by using a cross-linking molecule such as glutaraldehyde. The attachment may also be non-covalent and may involve simple adsorption of the binding molecule to the surface of the solid phase, such as polystyrene beads and microtiter plate. Labeling of binding molecules such as antibodies and other binding proteins are also well known in the prior art and are commonly called conjugation reactions and the labeled antibody is often called a conjugate. Typically, an amine-reactive moiety on the label reacts with an amine on the antibody to form an amide linkage. Other linkages, such as thioether, ester, carbamate, and the like between the antibody and the label may also be used.

The conditions for conducting an assay on a portion of a sample in accordance with the principles described herein may include carrying out the assay in an aqueous buffered medium at a moderate pH, generally that which provides optimum assay sensitivity. The aqueous medium may be solely water or may include from 0.1 to 40% by volume of a cosolvent. The pH for the medium may be in the range of 4 to 11, or 5 to 10, or 6.5 to 9.5, or 7 to 8. Usually, the pH value of the solution will be a compromise between optimum binding of the binding members of any specific binding pairs, the pH optimum for other reagents of the assay such as members of the signal producing system, and so forth. Various buffers may be used to achieve the desired pH and maintain the pH during the assay. Illustrative buffers include borate, phosphate, carbonate, TRIS, barbital, PIPES, HEPES, MES, ACES, MOPS, and BICINE, for example.

Various ancillary materials may be employed in the assay methods. For example, in addition to buffers, the composition, reagents, or reaction medium may comprise stabilizers for the medium and for the reagents employed. In some embodiments, the medium may comprise proteins (e.g., albumins), organic solvents (e.g., formamide), quaternary ammonium salts, polyanions (e.g., dextran sulfate), binding enhancers (e.g., polyalkylene glycols), polysaccharides (e.g., dextran, trehalose), blockers (e.g., blocker antibodies to prevent false positives), and combinations thereof.

Triggering the chemiluminescence of the analogs may be performed by the addition chemiluminescent triggering reagents. The chemiluminescent triggering reagents may be acidic or basic. Multiple chemiluminescent triggering reagents may be added sequentially. For example, an acidic solution may first be added followed by a basic solution. In some embodiments, the chemiluminescent triggering reagents comprise hydrogen peroxide, hydrogen peroxide salts, nitric acid, nitric acid salts, sodium hydroxide, ammonium salts, or combinations thereof.

### Conjugates

The present disclosure involves conjugates useful in high sensitive immunoassays for the detection of an analyte. Typically, these conjugates have a chemiluminescent acridinium conjugated to a carrier protein, wherein the carrier protein is conjugated to a antibody or antibody fragment (e.g., F(ab)) via a linker (e.g., PEG such as PEG₂-PEG₂₀ or PEG₃-PEG₁₀). In certain embodiments the conjugate comprises an excess of acridinium moieties (e.g., from 2-40, from 5-25) in relation to the carrier protein. In some embodiments, the first reacting step occurs in a medium comprising a buffer. In various implementations, the chemiluminescent acridinium compound is added in weight excess of the carrier protein (e.g., less than 100× excess or less than 50× excess or less than 40× excess or less than 30× excess or from 5× excess to 25× excess).

In some embodiments, the chemiluminescent acridinium is conjugated to the carrier protein by reacting the protein with a compound comprises a reactive functional group. For example, the compound may have the structure:

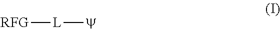

wherein RFG is a reactive functional group for conjugation to a protein,
L is absent (i.e., it is a bond) or a linker optionally comprising a group L^{C} or Z^{L}, and
ψ is a chemiluminescent acridinium comprising the structure:

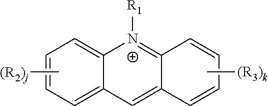

“j” and “k” are independently 0 (e.g., all R₂ groups are hydrogen, all R₃ groups are hydrogen), 1, 2, 3, or 4;
R¹ is hydrogen, —R, —X^{b}, —R^{L}—X^{b}, -L^{C}-R, -L^{C}-X^{b} (e.g., -L₁-X^{b}), —Z, —R^{L}—Z, -L^{C}-Z (e.g., -L₁-Z), or —R^{L}-L^{C}-R^{L}—Z (e.g., —R^{L}-L₁-R^{L}—Z);
R₂ and R₃ are independently selected at each occurrence from hydrogen, —R, an electron donating group, —X^{c}, —R^{L}—X^{c}, -L^{C}-X^{c} (e.g., -L₁-X^{c}), and —Z; wherein two vicinal R₂ or R₃ groups may together form a fused cyclic group (e.g., 5-7 membered fused aryl or heteroaryl group, 5-7 membered fused heterocyclic group) and wherein R₂ or R₃ may comprise a linkage to an imaging agent such as a fluorophore (e.g., rhodamine);
L^{C} is a divalent C₁₋₃₅ alkyl, alkenyl, alkynyl, aryl, or arylalkyl radical, optionally substituted (e.g., with 1 to 20 heteroatoms, with 1-20 substituents);
Z^{L} is a zwitterionic linker group having the structure:

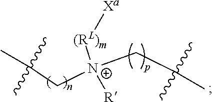

“m” is 0 (i.e. it is a bond) or 1;
“n” and “p” are independently at each occurrence an integer from 0 (i.e. it is a bond) to 10;
Z is a zwitterionic group independently at each occurrence has the structure:

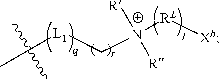

“q” and “l” are independently 0 or 1;
“r” is independently an integer from 0 to 10 (e.g., from 1 to 10, 1, 2, 3, 4, 5, 6, 7, 8, 9, 10);
X^{a} and X^{b} are independently at each occurrence an anionic group;
X^{c} is a protonated anionic group;
L₁ is independently at each occurrence —O—, —S—, —NH—, —N(R^{N})—, —(CH₂)₁₋₁₀—, —S(═O)₁₋₂—, —C═C—, —C═C—(CH₂)₁₋₃—, —C(O)—, —O—C(O)—, —C(O)—(CH₂)₁₋₄—, —(CH₂)₁₋₄—C(O)—, —C(O)—O—, —C(O)—N(R^{N})—, —C(O)—NH—, —N(R^{N})—C(O)—, —NH—C(O)—, —C(O)—N(R^{N})—(CH₂)₁₋₃—, —(CH₂)₁₋₃—C(O)—N(R^{N})—, —(CH₂)₁₋₃—N(R^{N})—C(O)—, —NH—S(O)₁₋₂—, —N(R^{N})—S(O)₁₋₂—, —S(O)₁₋₂—N(R^{N})—, —S(O)₁₋₂—NH—, —(CH₂)₁₋₃—NH—S(O)₁₋₂—, —(CH₂)₁₋₃—N(R^{N})—S(O)₁₋₂—, —(CH₂)₁₋₃—S(O)₁₋₂—N(R^{N})—, —(CH₂)₁₋₃—S(O)₁₋₂—NH—, —O—(CH₂)₁₋₄—, —(CH₂)₁₋₄—O—, —S—(CH₂)₁₋₄—, —(CH₂)₁₋₄—S—, —NH—(CH₂)₁₋₄—, —N(R^{N})—(CH₂)₁₋₄—, —(CH₂)₁₋₄—N(R^{N})—, —(OCH₂)₁₋₁₀—, —(CH₂O)₁₋₁₀—, —(OCH₂CH₂)₁₋₁₀—, or —(CH₂CH₂O)₁₋₁₀—;
R^{L} is independently at each occurrence a C₁₋₂₀ bivalent hydrocarbon radical (e.g., alkyl, alkenyl, aryl, phenyl, mono alkyl substituted phenyl, di alkyl substituted phenyl, alkynyl, arylalkyl), optionally having one or more (e.g., 1-10, 1-5) points of substitution (e.g., with 1-10 heteroatoms, with 1-10 substituents);
R is independently at each occurrence hydrogen or C₁₋₃₅ hydrocarbon (e.g., alkyl, alkenyl, alkynyl, or aralkyl) radical, optionally having one or more (e.g., 1-20, 1-10, 1-5) points of substitution (e.g., with 1-20 heteroatoms, with 1-20 substituents);
R′ and R″ are independently at each occurrence hydrogen or a C₁₋₁₀ alkyl;
R^{N} is independently at each occurrence from hydrogen or C₁₋₅ alkyl (e.g., methyl, ethyl, propyl); and
R′ is hydrogen or a C₁₋₁₀ alkyl; or
a salt thereof (e.g., a halide salt such as a chloride salt, a sulfonate salt such as a halosulfonate salt, a haloalkyl sulfonate salt a fluoroalkyl sulfonate salt, a carboxylate salt such as a haloalkyl carboxylate salt, fluoroalkyl carboxylate salt). For example, the chemiluminescent acridinium comprising a reactive functional group has the structure of formula (Ia):

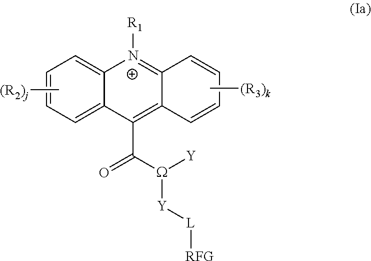

wherein Ω is O or N;
Y is selected from —R or —R^{L}—Z, or in the case where Ω is O then Y is absent; and
Y′ is either absent (i.e. it is a bond), or is selected from -L₁-, —R^{L}—, —R^{L}-L₁-, -L₁-L₁-, -L₁-R^{L}—, -L₁-R^{L}-L₁, and —R^{L}-L₁-R^{L}—. In some embodiments, the chemiluminescent acridinium comprising a reactive functional group has the structure of formula (Ib) or (Ic):

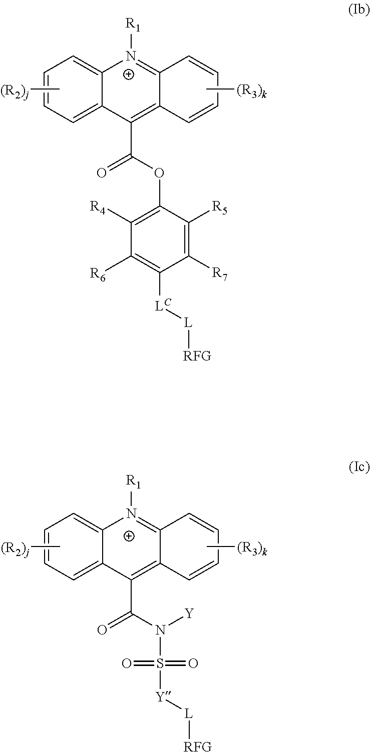

wherein R₄-R₇ are independently hydrogen, an electron donating group, or C₁₋₃₅ alkyl, alkenyl, alkynyl, aryl, alkoxy, alkylthio, or amino, and
Y″ is either absent (i.e., it is a bond) or -L^{C}-, -L₁-, —R^{L} or R^{L}-L-. In some embodiments, L is absent or C₁-C₅alkylene. In particular embodiments, the reactive functional group is an N-succinimidyl ester. In various implementations, at least one of R₂ and R₃ (e.g., at the C2 and/or C7 positions of the acridinium) are independently —X^{c}, —R^{L}—X^{c}, -L^{C}-X^{c} (e.g., -L₁-X^{c}). In some embodiments, at least one of R₂ and R₃ are independently alkoxy (e.g., C₁-C₄ alkoxy) substituted with —C(O)OH, —SO₂OH), —OSO₂OH), —OP(O)(OR^{P})OH, —OH, or combinations thereof.

When a moiety of the compounds of the present disclosure are described as comprising an analyte or a binding partner thereof in the context of molecular conjugates, a covalent linkage may be formed with an analyte or binding partner thereof (e.g., using the reactive functional groups which form covalent linkages) and the remaining portions of the conjugate. In such conjugation, the analyte or binding partner thereof may have a hydrogen on the unconjugated analyte or binding partner thereof that forms a covalent bond to the indicated moiety. The covalent linkage on the analyte or binding partner thereof may be formed, for example, at a group on the analyte, binding partner thereof, or derivatized version of the analyte containing a group for forming a linkage. The group may be, for example, an amine group, a thiol group, a carboxy group, a maleimidyl group, or a carbohydrate group. For example, if a covalent linkage is formed through a primary amine of the analyte or binding partner thereof, the compound may have the structure:

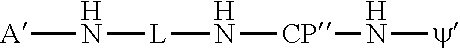

where L is a linker (e.g., PEG), CP is a carrier protein, ψ is an acridinium, and the unconjugated analyte or binding partner A (such an antibody or antibody fragment) has the structure A′-NH₂. Similarly, linker conjugation to the carrier protein may operate similarly such that the conjugated may have the structure:

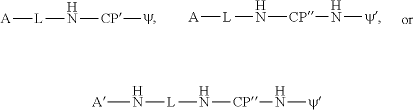

where L is independently a linker (e.g., PEG), ψ is an acridinium, A is an analyte or binding partner thereof (such an antibody or antibody fragment) and the carrier protein has the structure H₂N—CP′ or H₂N—CP″—NH₂ and ψ′ is the acridinium ester ψ having an amine reactive functional group reacted with an amine on the carrier protein. In some embodiments, the conjugation comprises an excess of acridinium esters as compared to the carrier protein (e.g., from 2 to 50).

In some embodiments, any hydrocarbon or substituted hydrocarbon disclosed herein (e.g., R, R′, R″, R^{N}, Y, Y′, Ω, L₁, L^{C}, R^{L}, R^{C}, R₁, R₂, R₂ₐ, R_{2b}, R_{2c}, R₃, R₄, R₅, R₆, R₇) may be substituted with one or more (e.g., from 1-6 or from 1-4 or from 1-3 or one or two or three) substituents X, where X is independently selected at each occurrence from one or more (e.g., 1-20) heteroatoms or one or more (e.g., 1-10) heteroatom-containing groups, or X is independently selected at each occurrence from —F, —Cl, —Br, —I, —OH, —OR*, —NH₂, —NHR*, —N(R*)₂, —N(R*)₃+, —N(R*)—OH, —N(→O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂—, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂-R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂; where, independently at each occurrence, R* may be H or a C₁₋₁₀ or C₁₋₈ or C₁₋₆ or C₁₋₄ hydrocarbon, including without limitation alkyl, alkenyl, alkynyl, aryl (e.g., phenyl), alkyl-aryl (e.g., benzyl), aryl-alkyl (e.g., tolyl) In some embodiments, X may comprise a C₁-C₈ or C₁-C₆ or C₂-C₄ perfluoroalkyl. In some embodiments, X may be a C₁-C₈ or C₂-C₆ or C₃-C₅ heterocycle (e.g., heteroaryl radical). The term “halo” or “halogen” refers to any radical of fluorine, chlorine, bromine or iodine. In some embodiments, X is independently selected at each occurrence from —OH, —SH, —NH₂, —N(R*)₂, —C(O)OR*, —C(O)NR*R*, —C(O)NR*R*, —C(O)OH, —C(O)NH₂, F, or —C₁. In some embodiments, X is F. R and R* may be, independently at each occurrence, saturated or unsaturated alkyl (e.g., C₁-C₈ alkyl). In some embodiments, R and R* are independently selected from hydrogen, methyl, ethyl, propyl, or isopropyl. In some embodiments, R and R* are independently selected from hydrogen, methoxy, ethoxy, propoxy, or isopropoxy. In some embodiments, X is —CF₃ or —O—CF₃.

L^{C} may have the structure:

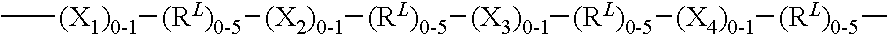

wherein X₁ is selected from ═N—, —O—, —S—, or —NR^{N}; X₂—X₄ are independently selected from —O—, —S—, —NR^{N}—, —C(O)—, —NR^{N}—C(O)—, —C(O)—NR^{N}—, —O—O(O)—, or —(O)—O—, —S—, —(O)—, or —(O)—S—; and
R^{L} is independently selected at each occurrence from —CH₂—, —(CH₂CH₂O)—, or —(OCH₂CH₂)—. In various embodiments, L^{C} comprises at least one atom (or at least two atoms) in the chain between A and ψ (or between A and Z^{L}).

Anionic groups, such as X^{a}, X^{b}, X^{c} may be, for example, independently at each occurrence carboxylate (—C(O)O—), sulfonate (—SO₃), sulfate (—OSO₃), phosphate (—OP(O)(OR^{P})O—), or oxide (—O—), and RP is hydrogen or C₁₋₁₂ hydrocarbon optionally having one or more (e.g., 1-10, 1-5) points of substitution (e.g., with 1-10 heteroatoms, with 1-10 substituents). Protonated versions of these groups (which may be protonated or in salt form to satisfy charge neutrality of a compound) include —C(O)OH, —SO₂OH), —OSO₂OH), —OP(O)(OR^{P})OH, or —OH, and RP is hydrogen or C₁₋₁₂ hydrocarbon optionally having one or more (e.g., 1-10, 1-5) points of substitution (e.g., with 1-10 heteroatoms, with 1-10 substituents) For example, R₁ may comprise (or be) —R^{L}—SO₃ (e.g., sulfopropyl). In some embodiments, R₁ comprises (or is) sulfopropyl. In some embodiments, R₁, R₂, and/or R₃ comprise (or are) sulfopropyl (which may be in zwitterionic formed (e.g., R₁) or neutral (e.g., R₂, R₃) form). In some embodiments, R¹ is —S(O)₂—NH—Z or —(CH₂)₁₋₃—S(O)₂—NH—Z. In various implementations, R₂ and R₃ are independently at each occurrence hydrogen, alkyl, or alkoxy (e.g., lower alkoxy such as C₁-C₄ alkoxy, methoxy, ethoxy, propoxy, isopropoxy) optionally substituted with —C(O)OH, —SO₂OH), —OSO₂OH), —OP(O)(OR^{P})OH, or —OH. In some embodiments, R₂ and R₃ are each hydrogen. In other embodiments, one of R₂ or R₃ is hydrogen and the other of R₂ or R₃ is alkoxy (e.g., lower alkoxy such as C₁-C₄ alkoxy, methoxy, ethoxy, propoxy, isopropoxy). In some embodiments, X^{a}, X^{b}, or X^{c} is sulfonate (—SO₃), m is 1, R^{L} is propyl, and n and p are each 3. For example, Z^{L} may have the structure:

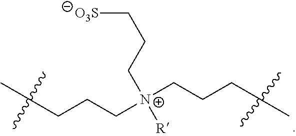

The compounds may be used to detect for the presence of a material in a sample such as an analyte (e.g., a biomolecule). In some embodiments, the analyte is a thyroid hormone (e.g., a thyroid stimulating hormone and, for example, the antibody is a binding partner therefor such as an anti-thyroid stimulating hormone monoclonal antibody (AntiTSH-mAb)), an androgen, a steroid hormone (e.g., androstenedione, testosterone), a troponin, thyroglobulin, anti-thyroid peroxidase antibody, triiodothyronine (T3) hormone, thyroxine (T4) hormone, thyroxine-binding globulin (TBG), neurofilament such as neurofilament light chain (e.g., serum neurofilament light chain), a vitamin (e.g., vitamin-D such as 25-hydroxy-vitamin D), or an antibody for a virus (e.g., hepatitis). In some embodiments, the biological sample is blood and the analyte must pass through the blood brain barrier to enter the blood (e.g., the analyte is neurofilament light chain). In these embodiments, the high sensitivity assay provided by the assays allows for even the low blood concentration levels of analytes.

In some embodiments, the compounds of the present disclosure may be zwitterionic and include one or more zwitterionic groups. For example, the R₁ group attached to the positively charged nitrogen of the acridinium may optionally substituted with up to 20 heteroatoms (e.g., N, O, S, P, Cl, Br, F) and therefore may in combination with the positively charged acridinium nitrogen atom, constitute a zwitterionic group. For example, a sulfopropyl or sulfobutyl group attached to the acridinium nitrogen may form a zwitterionic pair. The R₁ group may also be neutral (e.g., lower alkyl such as methyl) or by itself be zwitterionic (e.g., R¹ is —Z, —R^{L}—Z, -L₈-Z, or —R^{L}-L₈-R^{M}—Z). In some embodiments, R₁ has the structure:

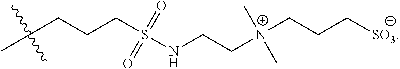

When the acridinium label is charged (e.g., R₁ has a net neutral charge), the compound may be in its salt form and optionally include a counterion to balance the positively charged nitrogen of the acridinium nucleus. The counterion may be selected from CH₃SO₄⁻, FSO₃⁻, CF₃SO₄⁻, C₄F₉SO₄⁻, CH₃C₆H₄SO₃⁻, halide (e.g., Cl⁻, F⁻, Br⁻), CF₃COO⁻, CH₃COO⁻, or NO₃⁻. In some embodiments, R₁ is methyl, ethyl, propyl, or isopropyl. In some embodiments, the acridinium compound may be zwitterionic by via covalent attachment to an anion. For example, R₁ may comprise —R^{L}—X^{a} and —X^{a} is sulfonate (—SO₃⁻). In some embodiments, R¹ is —R^{L}—X^{a} and —X^{a} is sulfonate (—SO₃⁻). In some embodiments, R¹ is —R^{L}—X or -L₈-Z. In some embodiments, L₈ is —S(O)₂—NH— or —(CH₂)₁₋₃—S(O)₂—NH—. R₁ may comprise a sulfopropyl group (—(CH₂)₃—SO₃⁻). In a specific embodiment, R¹ is sulfopropyl.

The substituents on the chemiluminescent acridinium ester may be modified to vary the rate and yield of light emission, to reduce the non-specific binding, increase stability, or increase hydrophilicity. Typically, these modifications will have minimal interference substantially with the binding of the analyte and its binding partner. Examples of substituent variability are disclosed in Natrajan et al. in U.S. Pat. No. 7,309,615, hereby incorporated by reference herein, which describes high quantum yield acridinium compounds containing electron donating groups such as alkoxy groups (OR*) at, for example, C2 and/or C7, wherein R* is a group comprising a sulfopropyl moiety or ethylene glycol moieties (e.g., —O(CH₂CH₂O)₀₋₅CH₃) or combinations thereof. In some embodiments, R₂ (e.g., R₂ₐ, R_{2b}, R_{2c}) and/or R₃ may be independently at each occurrence hydrogen an electron donating group such as an alkoxy group (e.g., OR such as —O(CH₂CH₂O)₀₋₅CH₃ and/or OR*.) or a group OG. Natrajan et al. in International Pub. No. WO2015/006174, hereby incorporated by reference in its entirety, also describes hydrophilic high quantum yield, chemiluminescent acridinium esters possessing certain electron-donating functional groups, for example, groups at the C2 and/or C7 positions as well. These electron donating groups (—OG) may have the structure:

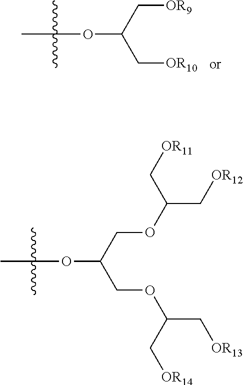

wherein R₉-R₁₄ are independently selected at each occurrence a methyl group or a group —(CH₂CH₂O)ₐCH₃, where a is an integer from 1 to 5. G may be independently selected at each occurrence from, for example, hydrogen, alkyl (e.g., C₁-C₄ alkyl), —(CH₂CH₂O)₁₋₁₀—OCH₃ such as —(CH₂CH₂O)₂—OCH₃ or —(CH₂CH₂O)₅—OCH₃.

ψ may comprise two flanking methyl groups on a phenolic ester to stabilize the bond as disclosed in Law et al. Journal of Bioluminescence and Chemiluminescence 4: 88-89 (1989), hereby incorporated by reference in its entirety. In some embodiments ψ in the conjugate has the structure:

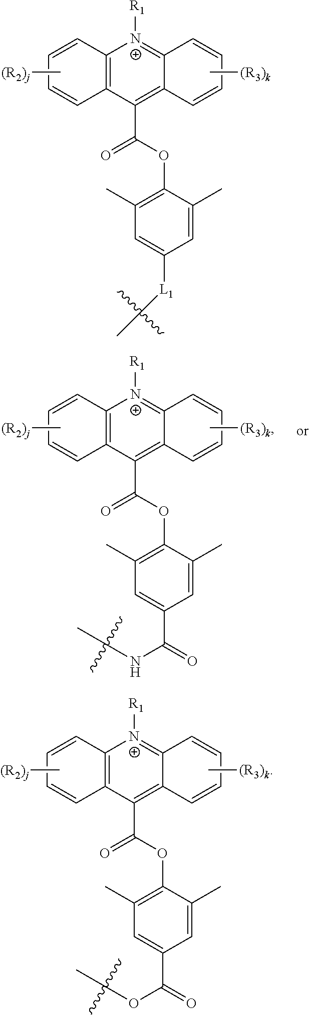

The reactive functional group may an amine-reactive group, a thiol-reactive group, a carboxy-reactive group, a maleimidyl-reactive group, or a carbohydrate-reactive group. In some embodiments, the reactive functional group may react with a functional group of the analyte or binding partner therefore such as a primary amine. The reactive functional group may comprise (or be) an isiothiocyanate, isocyantate, acyl azide, NHS ester, sulfonyl chloride, aldehyde, glyoxal, epoxide, oxirane, carbonate, aryl halide, imidoester, carbodiimide, anhydride, fluorophenyl ester, or combinations thereof. In various implementations, the reactive functional group labels the analyte or binding partner therefor through acylation or alkylation. For example, the linkage may be formed from a reactive functional group (RFG) selected from:

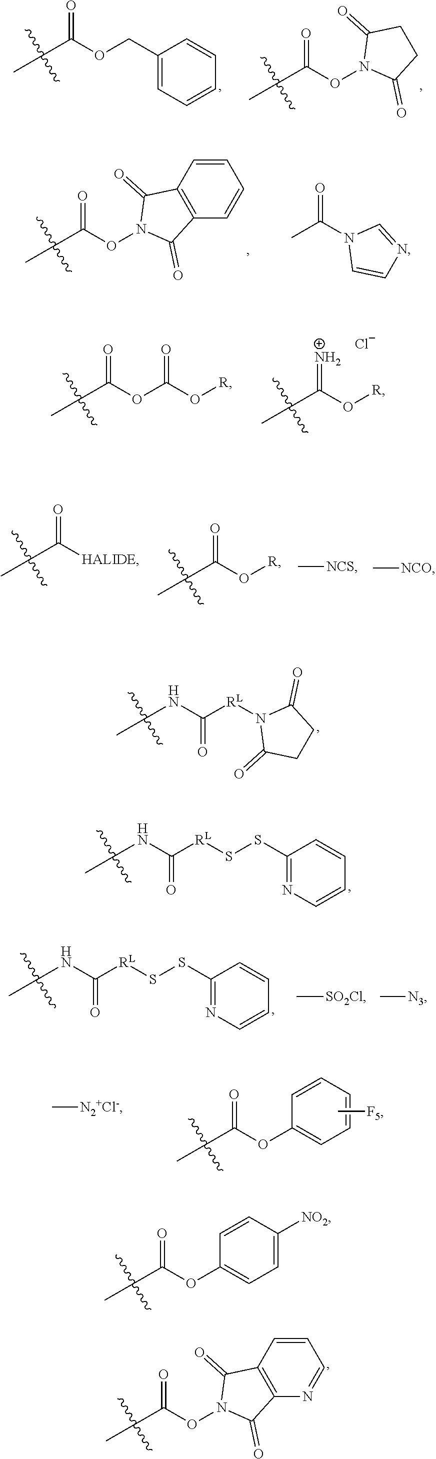

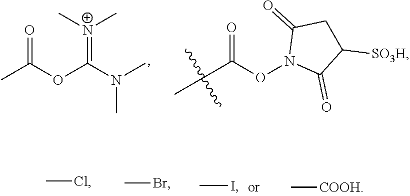

In some embodiments, the compound comprises a linker group having the structure —NH—C(O)— or —C(O)—NH—, —O(O)O—, or —OC(O)—. In a preferred embodiment, the compound or moiety thereof (e.g., L^{C}, Y) comprises at least one —NH—C(O)—, —C(O)—NH—, —O(O)O—, or —OC(O)— linker group.

The covalent linkage between RFG and Y (e.g., L) may comprise (or be) a divalent C₁₋₂₀ alkyl, alkenyl, alkynyl, aryl, or arylalkyl radical, optionally substituted with up to 20 heteroatoms (e.g., N, O, S, P, Cl, F, Br). In some embodiments L comprises a zwitterionic linker. L may have the structure -L^{C}-(Z^{L})_{z}—, wherein z is 0 or 1. L^{C} may have the structure

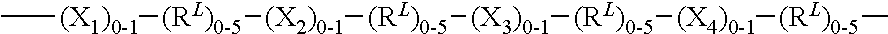

wherein X₁ is selected from —O—, —S—, —NR^{N}—, —C(O)—, —NR^{N}—C(O)—, —C(O)—NR^{N}—, —O—C(O)—, or —C(O)—O—, —S—C(O)—, or —C(O)—S—, ═N—, —O—, or —S—;
X₂—X₄ are independently selected from —O—, —S—, —NR^{N}—, —(O)—, —NR^{N}—C(O)—, —C(O)—NR^{N}—, —O—O(O)—, or —C(O)—, —S—C(O)—, or —C(O)—S—; and
R^{L} is independently selected at each occurrence from —CH₂—, —(CH₂CH₂O)—, or —(OCH₂CH₂)—; with, for example, the proviso that L^{C} comprises at least one atom (or at least two atoms) in the chain between A and ψ (or between A and Z^{L}).

In some embodiments, L and/or P comprises —C(O)—NH—. In some embodiments, L^{C} has the structure:

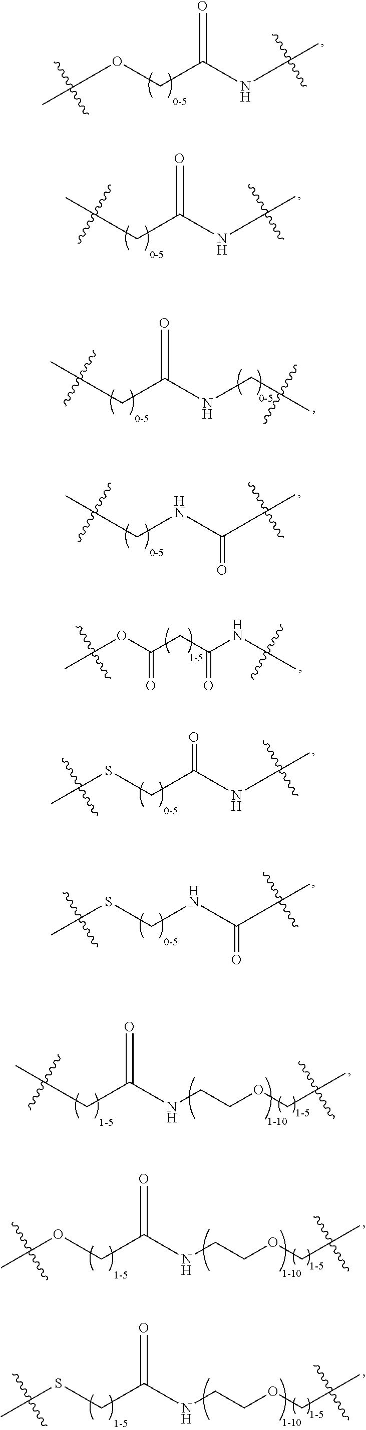

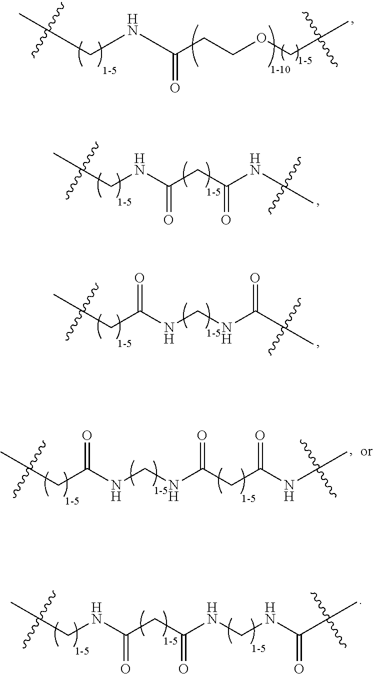

The detectable label may comprise a dimethyl acridinium ester (DMAE) moiety and a zwitterionic linker comprising a zwitterionic linker or a polyethylene glycol derived linker to improve properties of the compound. Such properties as non-specific binding, hydrophilicity, or compound stability may be improved when 4J comprises a zwitterionic linker or a polyethylene glycol derived linker or a dimethyl phenyl ester. In some embodiments, Z^{L} has the structure:

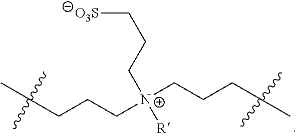

In several embodiments, R′ is hydrogen or lower alkyl (e.g., methyl, ethyl, propyl).

Chemiluminescence from multiple acridinium labels can be measured by using photomultiplier tubes (PMT), each of the PMT's which may be equipped with an optical filter that allows the light from an acridinium ester of interest to pass through while blocking the unwanted light. Yet another alternative detector is a charge-coupled device (CCD). The chemiluminescence may pass through a grating, such that the wavelength separation may occur along the detector (e.g., CCD detector) and the images can be analyzed accordingly.

The acridinium labels of U.S. Pat. No. 8,119,422, which is hereby incorporated by reference in its entirety, can be used as materials to form the wavelength separated and emission separated sets of acridinium labels.

The compounds of the present disclosure may be characterized by their stability. The acridinium labels used in the assay methods disclosed herein, such as the fast acridinium labels and/or the slow acridinium labels may be characterized as being stable. For example, a compound may be considered stable if there is a minimal loss of chemiluminescent activity as measured by the loss of relative light units (“RLU”) when the compounds or conjugates are stored in an aqueous solution typically, in the pH range of 6-9. Compounds having increased instability as compared to another compound may have a greater loss of chemiluminescent activity. For example, the compounds of the present disclosure may be characterized as having increased stabilities at pH 6 and/or 7 and/or 8) at 4° C. (common reagent storage temperature) and/or 37° C. (accelerated temperature) over 33 days. In some embodiments, the compounds may be characterized as having a change in chemiluminescent activity of less than (or from 1% to) 40% (e.g., less than 30%, less than 20%, from 10% to 40%, from 10% to 30%, from 10% to 20%) after 33 days of storage at 37° C. and pH 7 and/or pH 8.

The compounds can be prepared from commercially available starting materials, compounds known in the literature, or readily prepared intermediates, by employing standard synthetic methods (in addition to those provided herein). Standard synthetic methods and procedures for the preparation of organic molecules and functional group transformations and manipulations can be readily obtained from the relevant scientific literature or from standard textbooks in the field. It will be appreciated that where typical or preferred process conditions (e.g., reaction temperatures, times, mole ratios of reactants, solvents, pressures) are given, other process conditions can also be used unless otherwise stated. Optimum reaction conditions may vary with the particular reactants or solvent used, but such conditions can be determined by one skilled in the art by routine optimization procedures. Those skilled in the art of organic synthesis will recognize that the nature and order of the synthetic steps presented may be varied for the purpose of optimizing the formation of the compounds described herein.

Synthetic chemistry transformations (including protecting group methodologies) useful in synthesizing the compounds described herein are known in the art and include, for example, those such as described in R. C. Larock, Comprehensive Organic Transformations, 2d. Ed., Wiley-VCH Publishers (1999); P. G. M. Wuts and T. W. Greene, Protective Groups in Organic Synthesis, 4th Ed., John Wiley and Sons (2007); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof, each of which are hereby incorporated by reference in their entirety.

The processes described herein can be monitored according to any suitable method known in the art. For example, product formation can be monitored by spectroscopic means, such as nuclear magnetic resonance spectroscopy (e.g., ¹H or ¹³C), infrared spectroscopy (FT-IR), spectrophotometry (e.g., UV-visible), or mass spectrometry (MS), or by chromatography such as high-pressure liquid chromatography (HPLC) or thin layer chromatography (TLC).

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

Resolution of racemic mixtures of compounds can be carried out by any of numerous methods known in the art. For example, the absolute configuration of the stereoisomers may be determined by 1D and 2D NMR techniques such as COSY, NOESY, HMBC and HSQC. Specific implementations of these NMR techniques may be found in Hauptmann, H et al., Bioconjugate Chem. 11 (2000): 239-252 or Bowler, J. Steroids 54/1 (1989): 71-99, each hereby incorporated by reference in their entirety. Another example method includes preparation of the Mosher's ester or amide derivative of the corresponding alcohol or amine, respectively. The absolute configuration of the ester or amide is then determined by proton and/or ¹⁹F NMR spectroscopy. An example method includes fractional recrystallization using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid, or the various optically active camphorsulfonic acids. Resolution of racemic mixtures can also be carried out by elution on a column packed with an optically active resolving agent (e.g., dinitrobenzoylphenylglycine). Suitable elution solvent compositions can be determined by one skilled in the art.

Typically, zwitterionic acridinium esters (“ZAE”) comprising a reactive functional group for forming covalent linkages as described in U.S. Pat. No. 6,664,043 to Natrajan et al., U.S. Pat. No. 7,309,615 to Natrajan et al., U.S. Pat. No. 9,575,062 to Natrajan et al., or U.S. Pat. No. 9,487,480 to Natrajan, each hereby incorporated by reference in their entirety and in particular with respect to the zwitterionic acridinium esters described therein and their syntheses, may be used for synthesizing the compounds disclosed herein. For example, the zwitterionic acridinium ester starting materials may comprise an N-sulfopropyl (“NSP”) group in a zwitterionic moiety and/or comprise a charged nitrogen atom connected to the charged acridinium nucleus (“DIZAE”) and/or comprise a sterically stabilized dimethyl acridinium ester (“DMAE”) and/or comprise an isopropoxy functionalized aciridinium nucleus (“ISO”) and/or comprise a zwitterionic (“Z”) and/or hexa(ethylene) glycol derived (“HEG”) and/or glutarate derived (e.g., —C(O)—(CH₂)₃—C(O)—) linking moieties between the acridinium ester and the reactive functional group. The reactive functional group may by NH₂ or N-hydroxysuccinimidyl ester (“NHS”). For example, the compound (e.g., a compound for conjugating with an analyte or binding partner of an analyte such as a peptide, a protein, or a macromolecule including an antibody) may have the structure of formula (IV):

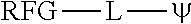

wherein RFG is a reactive functional group for conjugating to the analyte or binding partner for an analyte,
L is absent (i.e., it is a bond) or a linker, and
ψ is a chemiluminescent acridinium. The chemiluminescent conjugates or compounds for forming the conjugates may also be synthesized through the use of acridinium sulfonamide reactants. For example, the acridinium sulfonamides disclosed in U.S. Pat. No. 5,543,524 to Mattingly et al., hereby incorporated by reference in its entirety, are useful starting materials for the preparation of the chemiluminescent compounds disclosed herein.

The particle in the solid phase, and how it is prepared during the course of the assay may be important to achieve the desired resolution of the assay. The particles may have an average diameter of at least about 0.02 microns and not more than about 100 microns. In some embodiments, the particles have an average diameter from about 0.05 microns to about 20 microns, or from about 0.3 microns to about 10 microns. The particle may be organic or inorganic, swellable or non-swellable, and porous or non-porous. In a particular (but not limiting) embodiment, the particle has a density approximating water, generally from about 0.7 g/mL to about 1.5 g/mL, and is composed of material that can be transparent, partially transparent, or opaque. The particles can be comprised of organic and inorganic polymers, latex particles, magnetic or non-magnetic particles, and the like. In some non-limiting examples, the particles are chrome particles or latex particles.

The polymer particles can be formed of addition or condensation polymers. The particles can also be derived from naturally occurring materials, naturally occurring materials that are synthetically modified, and synthetic materials. Among organic polymers of particular interest are polysaccharides, particularly cross-linked polysaccharides, such as (but not limited to) agarose, which is available as Sepharose; dextran, which is available as Sephadex and Sephacryl; cellulose; starch; and the like; addition polymers, such as polystyrene, polyvinyl alcohol, homopolymers and copolymers of derivatives of acrylate and methacrylate, particularly (but not limited to) esters and amides having free hydroxyl functionalities, and the like.

The particles are typically readily dispersible in an aqueous medium and can be adsorptive or functionalizable so as to permit conjugation to monoclonal antibodies (or fragments thereof), either directly or indirectly through a linking group. When a linking group is utilized (in, for example, the particle or between the antibody fragment and the carrier protein), in some non-limiting embodiments, the linking group may comprise about 2 to about 50 atoms, or 4 to about 30 atoms, not counting hydrogen and may comprise a chain of from 2 to about 30 atoms, or 3 to about 20 atoms, each independently selected from the group normally consisting of carbon, oxygen, sulfur, nitrogen, and phosphorous. In some examples, the linking group comprises an oxime functionality.

The number of heteroatoms in the linking group may be in the range from 0 to about 20, or 1 to about 15, or about 2 to about 10. The linking group may be aliphatic or aromatic. When heteroatoms are present, oxygen is normally present as oxo or oxy, bonded to carbon, sulfur, nitrogen or phosphorous, nitrogen is normally present as nitro, nitroso or amino, normally bonded to carbon, oxygen, sulfur or phosphorous; sulfur is analogous to oxygen; while phosphorous is bonded to carbon, sulfur, oxygen or nitrogen, usually as phosphonate and phosphate mono- or diester. Common functionalities in forming a covalent bond between the linking group and the molecule to be conjugated are alkylamine, amidine, thioamide, ether, urea, thiourea, guanidine, azo, thioether and carboxylate, sulfonate, and phosphate esters, amides and thioesters.

For the most part, when a linking group has a linking functionality (functionality for reaction with a moiety which includes cross-linking functionality) such as, for example, a non-oxocarbonyl group including nitrogen and sulfur analogs, a phosphate group, an amino group, alkylating agent such as halo or tosylalkyl, oxy (hydroxyl or the sulfur analog, mercapto), oxocarbonyl (e.g., aldehyde or ketone), or active olefin such as a vinyl sulfone or α-, β-unsaturated ester, these functionalities are linked to amine groups, carboxyl groups, active olefins, alkylating agents, e.g., bromoacetyl. Where an amine and carboxylic acid or its nitrogen derivative or phosphoric acid are linked, amides, amidines, and phosphoramides are formed. Where mercaptan and activated olefin are linked, thioethers are formed. Where a mercaptan and an alkylating agent are linked, thioethers are formed. Where aldehyde and an amine are linked under reducing conditions, an alkylamine is formed. Where a ketone or aldehyde and a hydroxylamine (including derivatives thereof where a substituent is in place of the hydrogen of the hydroxyl group) are linked, an oxime functionality (═N—O—) is formed. Where a carboxylic acid or phosphate acid and an alcohol are linked, esters are formed. Various linking groups are provided in, for example, Cautrecasas, J. Biol. Chem. (1970): 245:3059, which is hereby incorporated by reference in its entirety and particularly in relation to linking groups.

### Sample

In some embodiments, a sample as disclosed herein is a biological sample. In some embodiments, a biological sample is a blood sample, e.g., drawn from an artery or vein of a subject. A blood sample can be a whole blood sample, a plasma sample, or a serum sample. In some embodiments, a biological sample includes CNS tissue.

In some embodiments, a sample is obtained from a subject. In some embodiments, a subject from which a sample was obtained is being assessed for neurological disease. In some embodiments, a subject from which a sample was obtained is suffering from or is at risk of developing neurological disease.

In some embodiments, a method disclosed herein includes obtaining a biological sample from a subject. In some embodiments, obtaining a biological sample from a subject includes drawing blood. In some embodiments, obtaining a biological sample from a subject includes performing a biopsy.

In some embodiments of methods disclosed herein, a sample is provided by, e.g., medical personnel.

### Subject

In some embodiments, a subject as disclosed herein is a mammal. In some embodiments, a mammal is a human.

### Methods Utilizing Thresholds

In some methods disclosed herein, a level of NfL can be compared to a threshold. In some embodiments, methods disclosed herein include a comparison of a level of NfL to respective thresholds. In some embodiments, methods disclosed herein include a comparison of a level of NfL to reference thresholds.

A reference threshold may be a threshold from a subject known or independently verified to have good CNS or neurological health, or from a subject known or independently verified to have poor CNS or neurological health, such as is the case for a subject having neurological disease. Alternately or in combination a subject's Biomarker profile is compared to a reference threshold determined from a plurality of subject of common known status (e.g., healthy, not diagnosed with neurological disease, or diagnosed with neurological disease). In some embodiments, a reference threshold is an average of known level of NfL from a plurality of subjects, or alternately is a range defined by the range of levels of NfL observed in reference subjects.

In more complex assessment approaches, a subject's Biomarker level is compared to a reference Biomarker level constructed from a larger number of subjects of a common status (e.g., healthy, not diagnosed with neurological disease, or diagnosed with neurological disease), such as at least 10, at least 50, at least 100, at least 500, at least 1000 or more subjects. Often, reference subjects are evenly distributed in status between (1) healthy/not diagnosed with neurological disease and (2) diagnosed with neurological disease. Assessment includes in some cases iterative or simultaneous comparison of a subject's biomarker (e.g, NfL) level to a plurality of profiles of known status.

A plurality of known reference Biomarker profiles (e.g., detected levels of NfL in a reference sample, demographic factors, and/or imaging-based biomarkers) can also be used to train a computational assessment algorithm, e.g., a machine learning model, such that a single comparison between a subject's Biomarker profile and a reference Biomarker profile provides an outcome that integrates or aggregates information from a large number of subjects of common known health status (e.g., healthy, not diagnosed with neurological disease, or diagnosed with neurological disease), such as at least 10, at least 50, at least 100, at least 500, at least 1000 or more individuals. Generation of such a reference Biomarker profile can facilitate much faster assessment of a subject's risk of neurological disease, or assessment using much less computational power.

A reference biomarker profile can be generated from a plurality of reference Biomarker profiles through any of a number of computational approaches known to one of skill in the art. Machine learning models are readily constructed, for example, using any number of statistical programming languages such as R, scripting languages such as Python and associated machine learning packages, data mining software such as Weka or Java, Mathematica, Matlab or SAS.

A subject's biomarker profile can be compared to a reference biomarker profile as generated above or otherwise by one of skill in the art, and an output assessment is generated. A number of output assessments are consistent with the disclosure herein. Output assessments include a single assessment, often narrowed by a sensitivity, specificity or sensitivity and specificity parameter, indicating a health status assessment (e.g., probability subject has neurological disease, subject is not at risk of neurological disease, subject is at risk of neurological disease, subject has neurological disease).

More specifically, in some embodiments, methods disclosed herein further include diagnosing a subject with neurological disease if the level of NfL that is detected is above a threshold value. In some embodiments, methods disclosed herein further include diagnosing a subject with neurological disease if the level of NfL that is detected is at least 1.3, at least 1.4, at least 1.5, at least 1.6, at least 1.7, at least 1.8, or at least 1.9 fold greater than a threshold value.

As discussed above, in some embodiments, methods of detecting NfL in a sample include contacting a sample with one or more antibody agents directed to NfL. In some embodiments, such methods also include contacting the sample with a first set of one or more detection agents. In some embodiments, the antibody agents are labeled with the first set of one or more detection agents. In some embodiments, the first set of one or more detection agents include one or more acridinium ester molecules.

Acridinium ester (AE) molecules can be used to label proteins and nucleic acids. Acridinium-labeled proteins can be used for detection in immunoassays. Exposing AE to an alkaline H₂O₂ (hydrogen peroxide) produces chemiluminscence. Light is emitted at a wavelength maximum in the range of 430 to 480 nm, depending on the specific AE variant. Such light can be detected, for example, by high-efficiency photomultiplier tubes.

In some embodiments, detecting binding between NfL and one or more antibody agents directed against NfL includes determining absorbance values or emission values for the first set of one or more detection agents. For example, the absorbance values are indicative of the level of binding (e.g., higher absorbance is indicative of more binding). In some embodiments, the absorbance values or emission values for the first set of one or more detection agents are above a threshold value. In some embodiments, the absorbance values or emission values for the first set of one or more detection agents is at least 1.3, at least 1.4, at least 1.5, at least 1.6, at least 1.7, at least 1.8, or at least 1.9 fold greater than a threshold value. In some embodiments, the threshold value is an average of absorbance values or emission values determined for a second set of one or more detection agents that label two or more control samples. In some such embodiments, the second set of one or more detection agents is similar to or the same as the first set of one or more detection agents.

In some embodiments, the methods disclosed herein further include diagnosing the subject with neurological disease if the level of NfL that is detected is above a threshold value. In some embodiments, the method includes diagnosing a subject with neurological disease if the level of NfL that is detected is at least 1.3, at least 1.4, at least 1.5, at least 1.6, at least 1.7, at least 1.8, or at least 1.9 fold greater than a threshold value.

In any of the above embodiments, a threshold value may be an average of values detected for two or more control samples. In some such embodiments, the values detected for the two or more control samples represent control levels for NfL. In some embodiments, the control samples include recombinant NfL. In some embodiments, the two or more control samples are each a sample obtained from a subject who does not have neurological disease. In some embodiments, the threshold value is a value reported in a standard table.

### Methods Utilizing Biomarker Profiles

An algorithm-based assay and associated information provided by the practice of any of the methods described herein can facilitate optimal treatment and decision making for subjects. For example, methods described herein can enable a physician or caretaker to identify patients who have a low likelihood of having neurological disease and therefore would not need treatment, would not need additional neurological tests, or would not need increased monitoring for neurological disease, or who have a high likelihood of having neurological disease, would need treatment, would need additional neurological tests.

A biomarker score can be determined by the application of a specific algorithm in some cases. In some embodiments, a biomarker score is quantitative. The algorithm used to calculate a biomarker score in the methods disclosed herein may group the expression level values of NfL or groups of biomarkers including NfL. The formation of a particular group of biomarkers, in addition, can facilitate the mathematical weighting of the contribution of various expression levels of biomarker or biomarker subsets (for example classifier) to the quantitative score.

Methods described herein, as well as kits and systems provided herein, can utilize an algorithm-based diagnostic assay for predicting if a subject from which the sample was obtained is at risk of or suffering from neurological disease, selecting a subject from which the sample was obtained for one or more neurological tests, and/or selecting a subject from which the sample was obtained to receive one or more doses of a neurological medication.

Methods disclosed herein include using a biomarker profile to compute a biomarker score. In some embodiments, using a biomarker profile to compute a biomarker score includes applying an algorithm to the Biomarker profile to compute a biomarker score. In some embodiments, an algorithm is or is derived from a decision tree methodology, a neural boosted methodology, a bootstrap forest methodology, a boosted tree methodology, a K nearest neighbors methodology, a generalized regression forward selection methodology, a generalized regression pruned forward selection methodology, a fit stepwise methodology, a generalized regression lasso methodology, a generalized regression elastic net methodology, a generalized regression ridge methodology, a nominal logistic methodology, a support vector machines methodology, a discriminant methodology, a naïve Bayes methodology, or a combination thereof. In some embodiments, an algorithm is or is derived from a decision tree methodology, a neural boosted methodology, a bootstrap forest methodology, a boosted tree methodology, a generalized regression lasso methodology, a generalized regression elastic net methodology, a generalized regression ridge methodology, a nominal logistic methodology, a support vector machines methodology, a discriminant methodology, or a combination thereof. In some embodiments, an algorithm is or is derived from a decision tree methodology, a neural boosted methodology, a bootstrap forest methodology, a boosted tree methodology, a support vector machines methodology, or a combination thereof.

A machine-learned algorithm may use data corresponding to one or more (e.g., two or more) demographic factors and a biomarker profile that includes data corresponding to a level of NfL in a sample from a subject.

In some embodiments, a method includes providing data corresponding to one or more demographic factors of a subject and a corresponding biomarker profile as input to a machine-learned algorithm. The corresponding Biomarker profile includes data for a level of NfL in a sample of a subject characterized by the demographic factor(s). Performance (e.g., predictive power, sensitivity and/or selectivity, and/or classification accuracy) of a machine-learned algorithm may be improved by using a machine-learned algorithm that considers a combination of NfL with one or more (e.g., two or more) demographic factors, as compared to a machine-learned algorithm that does not consider any demographic factor.

Additional algorithms can be used in methods provided herein, and the algorithms provided above are merely exemplary of the types of algorithms that can be used to generate a Biomarker score. Exemplary algorithms have been described, e.g., by Duda, 2001, Pattern Classification, John Wiley & Sons, Inc., New York. pp. 396-408 and pp. 411-412; and Hastie et al., 2001, The Elements of Statistical Learning, Springer-Verlag, New York, Chapter 9, each of which is hereby incorporated by reference. Moreover, as indicated above, combinations of algorithms can be used in methods provided herein. For example, a boosted tree methodology can be a combination of a decision tree methodology and a boosting methodology. Further combinations are possible and are contemplated for use in methods provided herein. Exemplary algorithms that can be used in the methods provided herein are described below.

### Decision Tree

One methodology that can be used to calculate a Biomarker score from a Biomarker profile is a decision tree. A decision tree can be constructed using a training population and specific data analysis algorithms. Decision trees are described generally by Duda, 2001, Pattern Classification, John Wiley & Sons, Inc., New York. pp. 395-396, which is hereby incorporated by reference. Tree-based methods partition the feature space into a set of rectangles, and then fit a model (like a constant) in each one.

A training population data can include Biomarker profiles (e.g., including a level of NfL in a sample) across a training set population. One specific algorithm that can be used to construct a decision tree is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests-Random Features,” Technical Report 567, Statistics Department, U. C. Berkeley, September 1999, which is hereby incorporated by reference in its entirety.

An aim of a decision tree is to induce a classifier (a tree) from real-world example data. This tree can be used to classify unseen examples that have not been used to derive the decision tree. As such, a decision tree can be derived from training data. Exemplary training data contains data for a plurality of subjects (e.g., a training population). A Biomarker profile can be provided and/or used for each respective subject. In some embodiments, training data includes Biomarker profiles for the training population.

Biomarker profiles may be used to train a machine-learning algorithm. Biomarker profiles may be from different subjects, a same subject at different times, or a combination thereof. Biomarker profiles may be associated with (e.g., labelled with) corresponding data indicative of health status. For example, Biomarker profile for a subject used as training data may be labelled with data indicative of health status for the subject. Such corresponding data may be a probability or a score (e.g., a Biomarker score). The health status may be a probability that a subject has neurological disease, whether or not a subject is or is not at risk of neurological disease, or whether or not a subject does or does not have neurological disease (e.g., is healthy, not diagnosed with neurological disease, or diagnosed with neurological disease). Health status may be determined manually, for example by a physician. Labelling Biomarker profiles with corresponding data indicative of health status may be performed manually (e.g., by the physician who determines health status).

Demographic factor data may be used in combination with biomarker profiles to train a machine-learning algorithm. For example, biomarker profile and data for one or more demographic factors for each of a set of subjects may be used to train a machine-learning algorithm. The set may be, for example, at least 10 subjects, at least 20 subjects, at least 50 subjects, at least 100 subjects, at least 200 subjects, at least 300 subjects, at least 500 subjects, at least 1,000 subjects, at least 1,500 subjects, at least 2,000 subjects, at least 5,000 subjects, or at least 10,000 subjects. In some embodiments, a combination of one or more demographic factors of age, sex, or both age and sex and a Biomarker profile for each of a set of subjects is used for training a machine-learning algorithm. The biomarker profile may include data corresponding to the level of NfL.

In some embodiments, training a machine-learning algorithm includes determining one or more feature values. Each feature value may correspond to a biomarker, e.g., NfL. One or more feature values may be determined based on biomarker profiles and/or a linear combination thereof. One or more feature values may correspond to a level of NfL. Training a machine-learning algorithm may include determining one or more decision rules based on Biomarker profiles used as training data. The one or more decision rules may be further based on one or more demographic factors corresponding to subject(s) that the biomarker profiles are for. The one or more decision rules may be based one or more feature values (e.g., determined during training). The one or more decision rules may be used in one or more decision trees.

Bagging, boosting, and additive trees can be combined with a decision methodology to improve weak decision rules. These techniques are designed for, and usually applied to, decision trees, such as the decision trees described above. In some embodiments, a machine-learned algorithm may include at least 25 decision trees, at least 50 decision trees, or at least 100 decision trees. In addition, such techniques can also be useful in decision rules developed using other types of data analysis algorithms such as linear discriminant analysis.

In bagging, a training set is sampled, generating random independent bootstrap replicates, constructing a decision rule on each of these, and aggregating them by a simple majority vote in the final decision rule. See, for example, Breiman, 1996, Machine Learning 24, 123-140; and Efron & Tibshirani, An Introduction to Boostrap, Chapman & Hall, New York, 1993, which is hereby incorporated by reference in its entirety.

In boosting, decision rules are constructed on weighted versions of the training set, which are dependent on previous classification results. Initially, all features under consideration have equal weights, and the first decision rule is constructed on this data set. Then, weights are changed according to the performance of the decision rule. Erroneously classified features get larger weights, and the next decision rule is boosted on the reweighted training set. In this way, a sequence of training sets and decision rules is obtained, which is then combined by simple majority voting or by weighted majority voting in the final decision rule. See, for example, Freund & Schapire, “Experiments with a new boosting algorithm,” Proceedings 13th International Conference on Machine Learning, 1996, 148-156, which is hereby incorporated by reference in its entirety.

Measurement data used in the methods, systems, kits and compositions disclosed herein are optionally normalized. Normalization refers to a process to correct for example, differences in the amount of genes or protein levels assayed and variability in the quality of the template used, to remove unwanted sources of systematic variation measurements involved in the processing and detection of genes or protein expression. Other sources of systematic variation are attributable to laboratory processing conditions.

In some instances, normalization methods are used for the normalization of laboratory processing conditions. Non-limiting examples of normalization of laboratory processing that may be used with methods of the disclosure include but are not limited to: accounting for systematic differences between the instruments, reagents, and equipment used during the data generation process, and/or the date and time or lapse of time in the data collection.

Assays can provide for normalization by incorporating the expression of certain normalizing standard genes or proteins, which do not significantly differ in expression levels under the relevant conditions, that is to say they are known to have a stabilized and consistent expression level in that particular sample type. Suitable normalization genes and proteins that can be used with the present disclosure include housekeeping genes. (See, for example, E. Eisenberg, et al., Trends in Genetics 19(7):362-365 (2003). In some applications, the normalizing biomarkers (genes and proteins), also referred to as reference genes, known not to exhibit meaningfully different expression levels in subjects with neurological disease as compared to control subjects without neurological disease. In some applications, it may be useful to add a stable isotope labeled standards which can be used and represent an entity with known properties for use in data normalization.

In other applications, a standard, fixed sample can be measured with each analytical batch to account for instrument and day-to-day measurement variability.

Machine learning algorithms for sub-selecting discriminating biomarkers and optionally subject characteristics, and for building classification models, are used in some methods and systems herein to determine clinical outcome scores. Examples of such algorithms are described above. These algorithms can aid in selection of important biomarker features and transform the underlying measurements into a score or probability relating to, for example, clinical outcome, disease risk, disease likelihood, presence or absence of disease, treatment response, and/or classification of disease status.

A machine-learned algorithm may output a Biomarker score. A machine-learned algorithm may determine if a subject is at risk of or is suffering from neurological disease. In some embodiments, an output of a machine-learned algorithm is a determination of whether a subject is at risk of or is suffering from neurological disease. A machine-learned algorithm may be a classifier for neurological disease. In some embodiments, a machine-learned algorithm can be used to classify whether or not a subject has neurological disease, for example based on a Biomarker profile for the subject. In some embodiments, the classifier or the classifying has a sensitivity and a specificity each of more than 80% (e.g., at least one or both of more than 90%).

A biomarker score can be determined by comparing a subject-specific Biomarker profile to a reference biomarker profile. A reference Biomarker profile can be representative a known diagnosis. For example, a biomarker profile can represent a positive diagnosis of neurological disease. As another example, a reference biomarker profile can represent a negative diagnosis of neurological disease. In some cases, an increase in a score indicates an increased likelihood of one or more of: a poor clinical outcome, good clinical outcome, high risk of disease, low risk of disease, complete response, partial response, stable disease, non-response, and recommended treatments for disease management. In some cases, a decrease in the quantitative score indicates an increased likelihood of one or more of: a poor clinical outcome, good clinical outcome, high risk of disease, low risk of disease, complete response, partial response, stable disease, non-response, and recommended treatments for disease management.

A similar biomarker profile from a subject to a reference biomarker profile often indicates an increased likelihood of one or more of: a poor clinical outcome, good clinical outcome, high risk of disease, low risk of disease, complete response, partial response, stable disease, non-response, and recommended treatments for disease management. In some applications, a dissimilar Biomarker profile between a subject and a reference indicates an increased likelihood of one or more of: a poor clinical outcome, good clinical outcome, high risk of disease, low risk of disease, complete response, partial response, stable disease, non-response, and recommended treatments for disease management.

Results can be provided to a subject, a health care professional or other professional. Results are optionally accompanied by a heath recommendation, such as a recommendation to confirm or independently assess neurological disease risk, for example using one or more neurological tests.

A recommendation optionally includes information relevant to a treatment regimen, such as information indicating that a treatment regimen. Efficacy of a regimen can be assessed in some cases by comparison of a subject's Biomarker profile at a first time point, optionally prior to a treatment and a later second time point, optionally subsequent to a treatment instance. Biomarker profiles can be compared to one another, each to a reference, or otherwise assessed so as to determine whether a treatment regimen demonstrates efficacy such that it should be continued, increased, replaced with an alternate regimen or discontinued because of its success in addressing neurological disease or associated signs and symptoms. Some assessments rely upon comparison of a subject's Biomarker profile at multiple time points, such as at least one time point prior to a treatment and at least one time point following a treatment. Biomarker profiles can be compared one to another or to at least one reference biomarker panel level or both to one another and to at least one reference biomarker panel level.

### Kits

Also provided by the present disclosure are kits including one or more anti-NfL agents and instructions for use (e.g., treatment, prophylactic, or diagnostic use). In some embodiments, the kit is used for an in vitro diagnostic assay to diagnose neurological disease. In some embodiments, the kits of the disclosure further include a neurological medication.

A kit according to the present disclosure typically involves an immunoassay reagent composition comprising an AE conjugated to an antibody (or fragment thereof) via a carrier protein. The kit may comprise accessory ingredients such a buffers, blocking reagents, ions, e.g. bivalent cations or monovalent cations, calibration proteins, secondary antibodies, detection reagent such as detection dyes and any other suitable compound or liquid necessary for the performance of analyte detection. Additionally, the kit may comprise an instruction leaflet and/or may provide information as to the relevance of the obtained results. In some embodiments, the kit further comprises the solid phase reagent. In some embodiments, the kit further comprises chemiluminescence triggering reagents.

A variety of immunoassay formats, including, for example, competitive and non-competitive immunoassay formats, antigen/analyte capture assays, and two-antibody sandwich assays can be used in accordance with the cartridges, kits, and methods described herein. The assay may be, for example, a competitive immunoassay which typically involves the detection of a large molecule, also referred to as macromolecular analyte, using binding molecules such as antibodies. The antibody is immobilized or attached to a solid phase such as a particle, bead, membrane, microtiter plate, or any other solid surface.

In an example of a competitive heterogeneous assay, a support having an antibody for an analyte (e.g., bovine monoclonal antibodies, mouse monoclonal antibodies, antibody fragments such as bovine antibody fragments, mouse antibody fragments) bound thereto is contacted with a medium containing a sample suspected of containing the analyte and the chemiluminescent conjugates (or “labeled analogs”) described herein. Analyte from the sample may compete for binding to the analyte antibody with the labeled analog. After separating the support and the medium, the label activity of the support or the medium is determined by conventional techniques and is related to the amount of analyte in the sample. In a variation of the above competitive heterogeneous assay, the support comprises the analyte analog, which competes with analyte of the sample for binding to an antibody reagent in accordance with the principles described herein. The labeled analyte analog may be covalently attached with a chemiluminescent or fluorescent molecule often referred to as a label or tracer.

When the solid phase with the immobilized antibody is mixed with a sample containing the analyte and the labeled analyte, a binding complex is typically formed between the analyte or the labeled analyte. This type of assay is often called a heterogeneous assay because of the involvement of a solid phase. The chemiluminescent signal associated with the binding complex can then be measured and the presence or absence of the analyte in the sample can be inferred. Usually, the binding complex is separated from the rest of the binding reaction components such as excess, labeled analyte, prior to signal generation. For example, if the binding complex is associated with a magnetic bead, a magnet can be used to separate the binding complex associated with the bead from bulk solution.

In an example of a sandwich assay format employing two antibodies (or fragments thereof), a solid support with a first immobilized antibody or fragment thereof for an analyte is mixed with a sample containing the analyte and a labelled conjugate comprising a second antibody or fragment thereof. A binding complex is formed between the solid particle and the labelled conjugate via the analyte in the sample. The signal associated with the binding complex and can the measured and the presence or absence or amount of analyte can be inferred. Usually, the binding complex is separated from the rest of the binding reaction components such as excess, labeled analyte, prior to signal generation. For example, if the binding complex is associated with a magnetic bead, a magnet can be used to separate the binding complex associated with the bead from bulk solution. In some embodiments, the first immobilized antibody is a biotinylated mouse monoclonal antibody bound to coated (e.g., streptavidin coated) optionally paramagnetic particles. In some embodiments, the second antibody is a mouse monoclonal antibody fragment labelled with acridinium (e.g., acridinium ester).

By using a series of “standards,” that is, known concentrations of the analyte, a “dose-response” curve can be generated for the known labeled analyte. These dose response curves may be identified individually for any acridinium label or identified based on combinations of the acridinium labels used in the assay. Thus, the dose-response curve correlates a certain amount of measured signal with a specific concentration of analyte. In a competitive assay, as the concentration of the analyte increases, the amount of signal decreases if the chemiluminescence from the binding complex is measured. The concentration of the analyte in an unknown sample can then be calculated by comparing the signal generated by an unknown sample containing the macromolecular analyte, with the dose-response curve.

The methodology of the attachment of binding molecules such as antibodies to solid phases typically involves a mixing of the requisite components to induce attachment. For example, an antibody can be covalently attached to a particle containing amines on its surface by using a cross-linking molecule such as glutaraldehyde. The attachment may also be non-covalent and may involve simple adsorption of the binding molecule to the surface of the solid phase, such as polystyrene beads and microtiter plate. Labeling of binding molecules such as antibodies and other binding proteins are also well known in the prior art and are commonly called conjugation reactions and the labeled antibody is often called a conjugate. Typically, an amine-reactive moiety on the label reacts with an amine on the antibody to form an amide linkage. Other linkages, such as thioether, ester, carbamate, and the like between the antibody and the label may also be used.

In some embodiments, the one or more anti-NfL agents include antibody agents. In some embodiments, one or more, two or more, three or more, four or more, five or more, six or more of the antibody agents are labeled with a detectable moiety. In some embodiments, the kit further includes a detection agent (e.g., one or more acridinium ester molecules). In some embodiments, one or more of the antibody agents are labeled with one or more of the acridinium ester molecules. In some embodiments, the kit further includes one or more secondary antibody agents that specifically bind to one or more of the anti-NfL antibody agents.

In some embodiments, the one or more anti-NfL agents include nucleic acid probes. In some embodiments, at least a portion of each nucleic acid probe hybridizes to one or more portions of a nucleotide that encodes NfL. Nucleotides that encode NfL can be DNA (e.g., cDNA) or RNA (e.g. mRNA). In some embodiments, the nucleic acid probes are labeled with one or more detection agents (e.g., wherein the detection agents indicate presence of nucleotides that encode NfL).

In some embodiments, the kit further includes one or more control samples. In some embodiments, the control samples include one or more standards. In some embodiments, a standard includes recombinant NfL. In some embodiments, a standard includes synthetic NfL nucleic acids.

In addition to the above, a kit can include other ingredients, such as a solvent or buffer, a stabilizer or a preservative, and/or an agent for treating a condition or disorder described herein. Alternatively, other ingredients can be included in a kit, but in different compositions or containers than the anti-NfL agents. In such embodiments, a kit can include instructions for admixing the anti-NfL agents and the other ingredients, or for using the anti-NfL together with the other ingredients.

In certain embodiments, kits for use in accordance with the present disclosure may include, a reference or control sample(s), instructions for processing samples, performing tests on samples, instructions for interpreting the results, buffers and/or other reagents necessary for performing tests.

The present disclosure also provides that recognition that certain single biomarkers can be helpful for detecting and/or diagnosing neurological disease. The present disclosure further provides the insight that NfL is especially useful for detecting and/or diagnosing neurological disease or condition. Thus, methods, compositions, and kits described herein can be used for assays to assess the risk of neurological disease, assess whether a subject should undergo further neurological tests, and/or diagnose neurological disease based on detection or measurement of NfL in a sample, e.g., a biological sample obtained from a subject.

Methods and kits provided herein are able to detect neurological disease in a sample with a sensitivity and a specificity that renders the outcome of the test reliable enough to be medically actionable. Methods and kits described herein for detection and/or diagnosis of neurological disease in a subject detects neurological disease with a sensitivity greater than 75%, greater than 80%, greater than 85%, greater than 90%, greater than 95%, greater than 96%, greater than 97%, greater than 98%, greater than 99%, or about 100%. In some embodiments, methods and kits provided herein can detect neurological disease with a sensitivity that is between about 70%-100%, between about 80%-100%, or between about 90-100%. In some embodiments, methods and kits provided herein can detect neurological disease with a specificity greater than 70%, greater than 75%, greater than 80%, greater than 85%, greater than 90%, greater than 95%, greater than 96%, greater than 97%, greater than 98%, greater than 99%, or about 100%. In some embodiments, methods and kits provided herein can detect neurological disease with a specificity that is between about 50%-100%, between about 60%-100%, between about 70%-100%, between about 80%-100%, or between about 90-100%. In some embodiments, methods and kits provided herein can detect neurological disease with a sensitivity and a specificity that is 50% or greater, 60% or greater, 70% or greater, 75% or greater, 80% or greater, 85% or greater, 90% or greater. In some embodiments, methods and kits provided herein can detect neurological disease with a sensitivity and a specificity that is between about 50%-100%, between about 60%-100%, between about 70%-100%, between about 80%-100%, or between about 90-100%.

### Compositions

Also provided herein are compositions. In some embodiments, a composition includes NfL and one or more anti-NfL agents. In some embodiments, one or more anti-NfL agents in a composition provided herein include antibody agents. In some embodiments, one or more of the antibody agents are labeled with a detectable moiety. In some embodiments, the kit further includes a detection agent (e.g., one or more acridinium ester molecules). In some embodiments, one or more of the antibody agents are labeled with one or more of the acridinium ester molecules. In some embodiments, the kit further includes one or more secondary antibody agents that specifically bind to one or more of the anti-NfL antibody agents.

In some embodiments, one or more anti-NfL agents in a composition provided herein include nucleic acid probes. In some embodiments, at least a portion of each nucleic acid probe hybridizes to one or more portions of a nucleotide that encodes NfL. Nucleotides that encode NfL can be DNA (e.g., cDNA) or RNA (e.g. mRNA). In some embodiments, the nucleic acid probes are labeled with one or more detection agents (e.g., wherein the detection agents indicate presence of nucleotides that encode NfL).

In some embodiments, a composition includes one or more control samples. In some embodiments, the control samples include one or more standards. In some embodiments, a standard includes recombinant NfL. In some embodiments, a standard includes synthetic NfL nucleic acids.

In addition to the above, a composition can include other ingredients, such as a solvent or buffer, a stabilizer or a preservative, and/or an agent for treating a condition or disorder described herein.

In some embodiments, the present disclosure includes an Atellica® IM Serum Neurofilament Light Chain (sNfL) assay for use in the quantitative measurement of Neurofilament Light Chain in human serum and plasma (EDTA) using the Atellica® IM Analyzer.

In another aspect of the invention, a reagent may be provided for the detection of an analyte comprising a chemiluminescent acridinium compound bound the analyte or binding partner. The reagent may comprise from 0.1 to 100 ng/mL of the chemiluminescent acridinium compound or from 1 to 50 ng/mL of the chemiluminescent acridinium compound or from 5 to 30 ng/mL of the chemiluminescent acridinium compound. In some embodiments, the compound is provided in a reagent which further comprises a buffer.

Neurofilaments are proteins that are exclusively expressed in the cytoplasm of neurons. Their main function is formation of the neuronal cytoskeleton. There are five known neurofilament subunits, that can be differentiated by their molecular mass. Beside Neurofilament light (NfL), medium (NfM) and heavy (NfH) with molecular masses of approximately 70, 160 and 200 kilo Dalton (kDa), Peripherin and Internexin with a molecular mass of 56 and 66 kDa exist. Due to their stability and role in the neuronal structure they gained interest in the usage as marker for axonal damage. During neurodegeneration, high concentrations of neurofilaments are released into the blood or cerebrospinal fluid where they can be detected.

In embodiments, the sNfL assay of the present disclosure is a fully automated 2-step sandwich immunoassay using acridinium ester chemiluminescent technology. In embodiments, the assay includes two anti-sNfL antibodies. In embodiments, the first antibody, is a lite reagent characterized as a mouse monoclonal anti-sNfL antibody labeled with acridinium ester. In embodiments, the second antibody is a biotinylated mouse monoclonal anti-sNfL antibody that is bound to streptavidin-coated paramagnetic microparticles in a solid phase. In embodiments, a direct relationship exists between the amount of sNfL present in the patient or subject sample and the amount of relative light units (RLUs) detected by the system. In embodiments, a solid phase includes streptavidin-coated paramagnetic microparticles (about 0.5 mg/mL) with biotinylated mouse monoclonal anti-human NfL antibody (about 10 microgram/mL) in buffer; sodium azide (less than 0.1%); blockers (e.g., bovine); surfactant; preservatives, and the like. In embodiments, a lite reagent includes mouse monoclonal anti-human NfL antibody (about 0.3 microgram/mL) labeled with acridinium ester in buffer sodium azide (less than 0.1%); blockers (mouse, bovine); surfactants and preservatives. In embodiments, a calibrator is included such as a 1.0 mL vial; lyophilized composition. In embodiments, after reconstitution of the calibrator, compositions include low and high levels of sNfL antigen in human serum, sodium azide (less than 0.1%); and preservatives. In embodiments, the calibrants are lyophilized and stored at 2-8 degrees Celsius. In embodiments, a solution or mixture including a quality control composition or solution, e.g., including low and high levels of sNfL antigen in human serum, sodium azide (less than 0.1%); and preservatives. In embodiments, a kit further includes a diluent. In embodiments, a diluent includes buffer, sodium azide, blockers (bovine) surfactant and preservatives.

In embodiments, the present disclosure includes: a system capable of or configured to automatically performs the following steps: dispensing about 100 μL of sample into a cuvette; dispensing about 100 μL of lite reagent, and then incubating for about 32 minutes at about 37° C.; dispensing about 100 μL of solid phase as described herein, then incubating for about 17 minutes at about 37° C.; performing a wash sequence using Atellica IM wash or other suitable wash solution; dispensing about 300 pL each of Atellica IM acid reagent and Atellica IM base reagent or an acid and base in an amount sufficient to initiate the chemiluminescent reaction; and optionally reporting results.

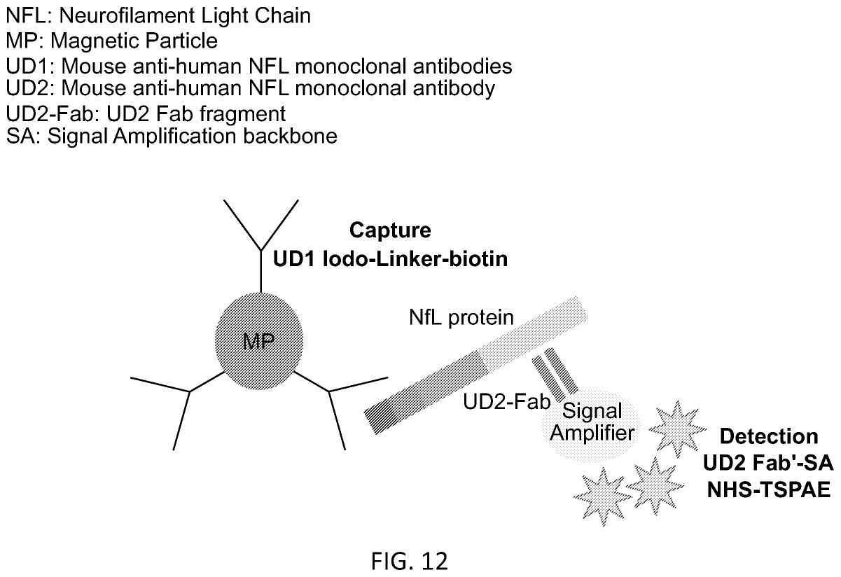

In embodiments, an assay or kit of the present disclosure includes one or more components or features depicted in FIG. 12. For example, a suitable assay in accordance with the present disclosure includes one or neurofilament light chains, one or more magnetic particles, one or more mouse anti-human NFL monoclonal antibodies, one or more mouse anti-human NFL monoclonal antibodies, and one or more Fab fragments as shown and described herein. In embodiments, the assay includes a signal amplifier. In embodiments the signal amplifier is characterized as a binding partner to the NfL protein. In embodiments, the signal amplifier is albumin. In embodiments, the signal amplifier includes bovine serum albumin. In embodiments, the signal amplifier includes one or more signal generating moieties. In embodiments, the signal amplifier includes one or more of albumin, dendrimer, nanoparticles, and combinations thereof. In embodiments, the signal amplifier includes one or more, such as 3, 4, 5, 6, 7, 8, 9, 10, or more signal generating moieties.

### Computer Systems

Methods described herein can be implemented in a computer system having a processor that executes specific instructions in a computer program. In some embodiments, a computer system may be arranged to output a biomarker score based on receiving a biomarker profile and/or a level of NfL, demographic factors, and/or imaging-based biomarkers. Particularly, a computer program may include instructions for the system to select appropriate next steps, including additional medication, a treatment, and/or additional testing (e.g., neurological tests) for a subject.

In some embodiments, the computer program may be configured such that the computer system can identify a subject for further testing (e.g., neurological tests), identify a subject as being at risk or having neurological disease, and/or identify a subject to receive medication based on received data (e.g., biomarker profile) and use the data to calculate a biomarker score. A system may be able to rank-order identified next steps based on biomarker profile with demographic factors and/or imaging-based biomarkers. A system may be able to adjust the rank ordering based on, e.g., a clinical response of a subject or of a family member of the subject who has or is suspected of having neurological disease.

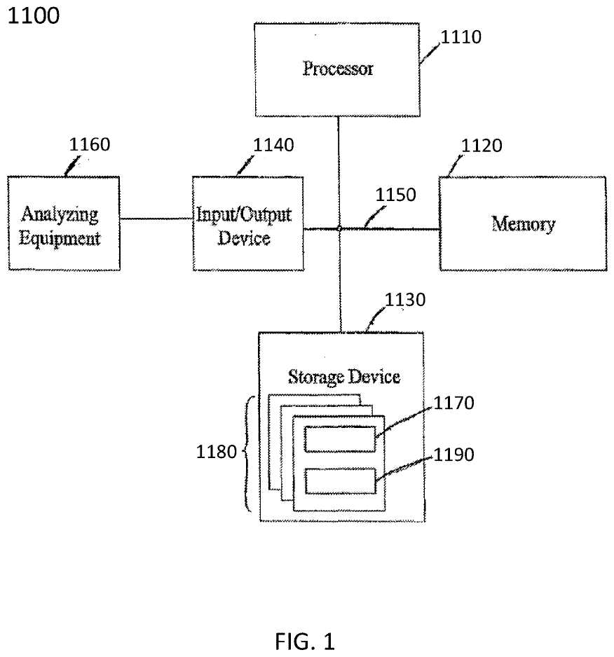

FIG. 1 is a block diagram of a computer system 1100 that can be used in the operations described above, according to one embodiment. The system 1100 includes a processor 1110, a memory 1120, a storage device 1130 and an input/output device 1140. Each of the components 1110, 1120, 1130 and 1140 are interconnected using a system bus 1150. The system may include analyzing equipment 1160 for determining a level of NfL in a sample.

The processor 1110 is capable of processing instructions for execution within the system 1100. In one embodiment, the processor 1110 is a single-threaded processor. In another embodiment, the processor 1110 is a multi-threaded processor. The processor 1110 is capable of processing instructions stored in the memory 1120 or on the storage device 1130, including for receiving or sending information through the input/output device 1140.

The memory 1120 stores information within the system 1100. In one embodiment, the memory 1120 is a computer-readable medium. In one embodiment, the memory 1120 is a volatile memory unit. In another embodiment, the memory 1120 is a non-volatile memory unit.

The storage device 1130 is capable of providing mass storage for the system 1100. In one embodiment, the storage device 1130 is a computer-readable medium.

The input/output device 1140 provides input/output operations for the system 1100. In one embodiment, the input/output device 1140 includes a keyboard and/or pointing device. In one embodiment, the input/output device 1140 includes a display unit for displaying graphical user interfaces.

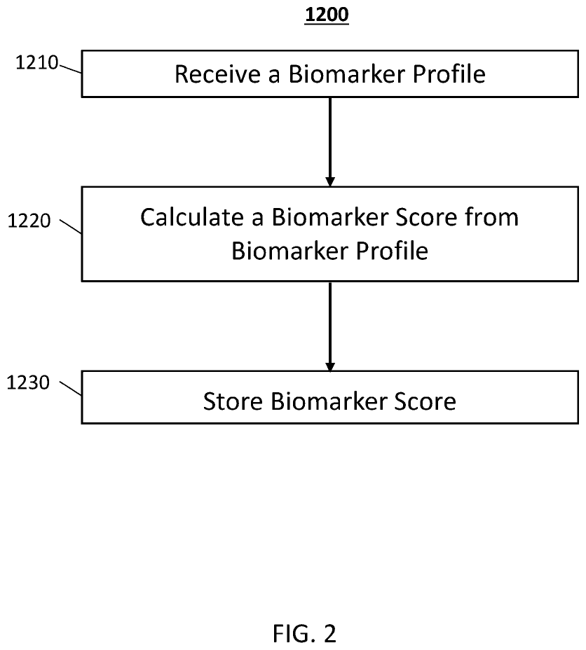

The system 1100 can be used to build a database. FIG. 2 shows a flow chart of a method 1200 for building a database for use in identifying a subject for further testing (e.g., neurological tests), identifying a subject as being at risk or having neurological disease, and/or identifying a subject to receive medication. Preferably, a method 1200 is performed in a system 1100. For example, a computer program product can include instructions that cause a processor 1110 to perform the steps of a method 1200 or a method 1300.

Method 1200 includes the following steps. Receiving, in step 1210, an subject's biomarker Profile (e.g., levels of NfL in a sample). A computer program in the system 600 may include instructions for presenting a suitable graphical user interface on input/output device 640, and the graphical user interface may prompt the user to enter the levels 670 using the input/output device 640, such as a keyboard. Calculating, in step 1220, a Biomarker score from the biomarker profile. As described herein, calculating, in step 1220, a biomarker score from (i) a biomarker profile and (ii) demographic factors and/or imaging-based biomarkers. Storing, in step 1230, a biomarker score. The system 600 may store a Biomarker score in the storage device 630. Additionally or alternatively, a system 600 may provide a readout including a biomarker score. A readout may also include proposed next steps for a subject and/or a confidence level associated with a biomarker score.

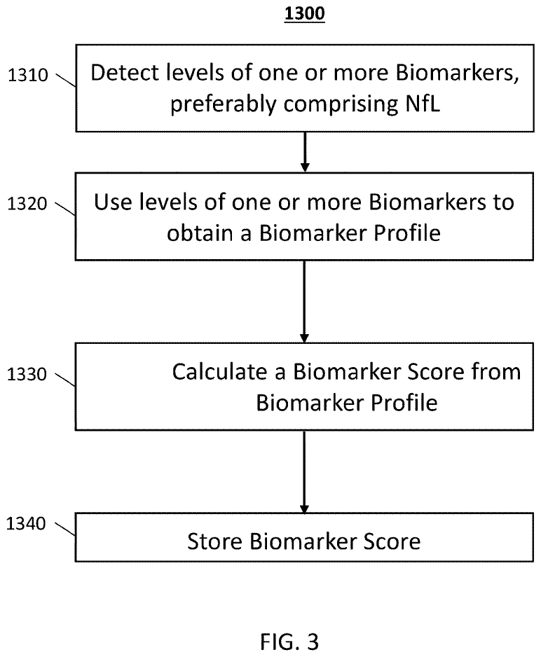

As shown in FIG. 3, method 1300 includes the following steps. Detecting, in step 1310, levels of NfL in a sample, e.g., from a subject. Using, in step 1320, levels of NfL to obtain a Biomarker Profile. Calculating, in step 1330, a biomarker score from a biomarker profile. As described herein, calculating, in step 1330, a biomarker score from (i) a biomarker profile and (ii) demographic factors and/or imaging-based biomarkers. Storing, in step 1340, a biomarker score. The system 600 may store a Biomarker score in the storage device 630. Additionally or alternatively, a system 600 may provide a readout including a biomarker score. A readout may also include proposed next steps for a subject and/or a confidence level associated with a Biomarker score.

Additionally, non-transitory computer readable media containing executable instructions that when executed cause a processor to perform operations including a method as provided herein are provided. For example, a non-transitory computer readable medium containing executable instructions that when executed cause a processor to perform operations including a method of 1200 or 1300 described above.

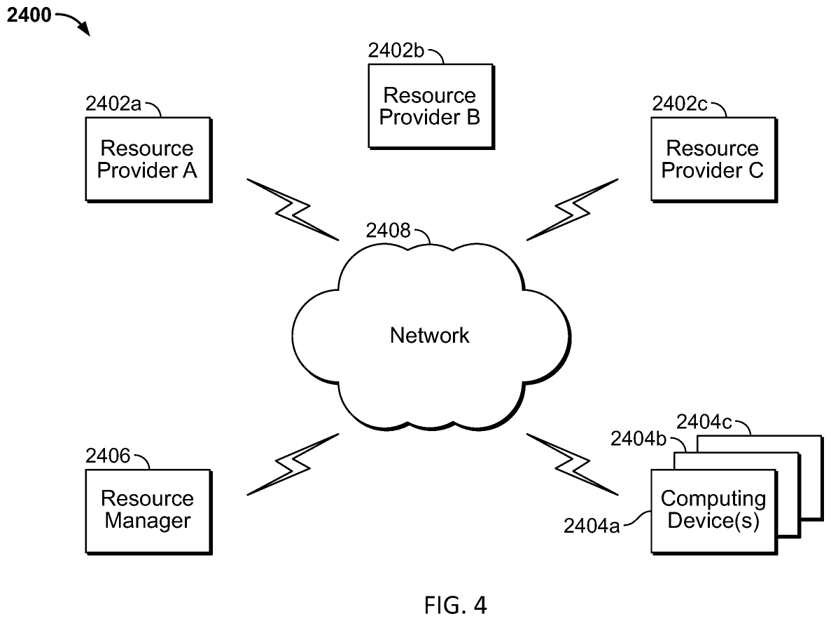

Illustrative embodiments of systems and methods disclosed herein were described above with reference to computations performed locally by a computing device. However, computations performed over a network are also contemplated. FIG. 4 shows an illustrative network environment 2400 for use in the methods and systems described herein. In brief overview, referring now to FIG. 4, a block diagram of an illustrative cloud computing environment 2400 is shown and described. The cloud computing environment 2400 may include one or more resource providers 2402a, 2402b, 2402c (collectively, 2402). Each resource provider 2402 may include computing resources. In some implementations, computing resources may include any hardware and/or software used to process data. For example, computing resources may include hardware and/or software capable of executing algorithms, computer programs, and/or computer applications. In some implementations, illustrative computing resources may include application servers and/or databases with storage and retrieval capabilities. Each resource provider 2402 may be connected to any other resource provider 2402 in the cloud computing environment 2400. In some implementations, the resource providers 2402 may be connected over a computer network 2408. Each resource provider 2402 may be connected to one or more computing device 2404a, 2404b, 2404c (collectively, 2404), over the computer network 2408.

The cloud computing environment 2400 may include a resource manager 2406. The resource manager 2406 may be connected to the resource providers 2402 and the computing devices 2404 over the computer network 2408. In some implementations, the resource manager 2406 may facilitate the provision of computing resources by one or more resource providers 2402 to one or more computing devices 2404. The resource manager 2406 may receive a request for a computing resource from a particular computing device 2404. The resource manager 2406 may identify one or more resource providers 2402 capable of providing the computing resource requested by the computing device 2404. The resource manager 2406 may select a resource provider 2402 to provide the computing resource. The resource manager 2406 may facilitate a connection between the resource provider 2402 and a particular computing device 2404. In some implementations, the resource manager 2406 may establish a connection between a particular resource provider 2402 and a particular computing device 2404. In some implementations, the resource manager 2406 may redirect a particular computing device 2404 to a particular resource provider 2402 with the requested computing resource.

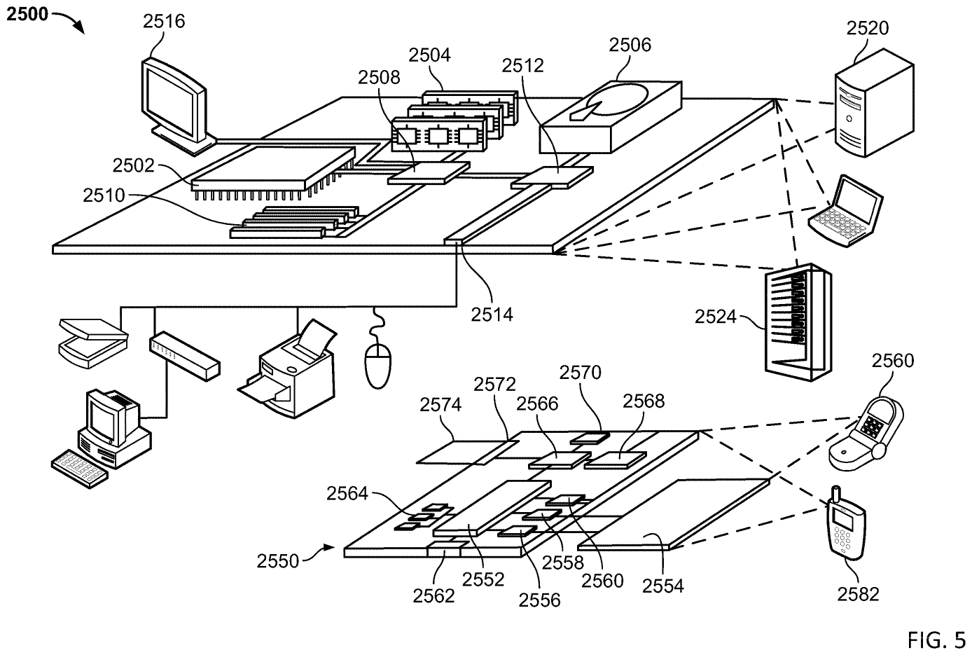

FIG. 5 shows an example of a computing device 2500 and a mobile computing device 2350 that can be used in the methods and systems described in this disclosure. The computing device 2500 is intended to represent various forms of digital computers, such as laptops, desktops, workstations, personal digital assistants, servers, blade servers, mainframes, and other appropriate computers. The mobile computing device 2350 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 2500 includes a processor 2502, a memory 2504, a storage device 2506, a high-speed interface 2508 connecting to the memory 2504 and multiple high-speed expansion ports 2310, and a low-speed interface 2312 connecting to a low-speed expansion port 2314 and the storage device 2506. Each of the processor 2502, the memory 2504, the storage device 2506, the high-speed interface 2508, the high-speed expansion ports 2310, and the low-speed interface 2312, are interconnected using various busses, and may be mounted on a common motherboard or in other manners as appropriate. The processor 2502 can process instructions for execution within the computing device 2500, including instructions stored in the memory 2504 or on the storage device 2506 to display graphical information for a GUI on an external input/output device, such as a display 2316 coupled to the high-speed interface 2508. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (e.g., one or more processors) of any number of computing devices (e.g., one or more computing devices). Furthermore, where a function is described as being performed by “a processor”, this encompasses embodiments wherein the function is performed by any number of processors (e.g., one or more processors) of any number of computing devices (e.g., one or more computing devices) (e.g., in a distributed computing system).

The memory 2504 stores information within the computing device 2500. In some implementations, the memory 2504 is a volatile memory unit or units. In some implementations, the memory 2504 is a non-volatile memory unit or units. The memory 2504 may also be another form of computer-readable medium, such as a magnetic or optical disk.

The storage device 2506 is capable of providing mass storage for the computing device 2500. In some implementations, the storage device 2506 may be or contain a computer-readable medium, such as a hard disk device, an optical disk device, a flash memory or other similar solid state memory device, or an array of devices, including devices in a storage area network or other configurations. Instructions can be stored in an information carrier. The instructions, when executed by one or more processing devices (for example, processor 2502), perform one or more methods, such as those described above. The instructions can also be stored by one or more storage devices such as computer- or machine-readable mediums (for example, the memory 2504, the storage device 2506, or memory on the processor 2502).

The high-speed interface 2508 manages bandwidth-intensive operations for the computing device 2500, while the low-speed interface 2312 manages lower bandwidth-intensive operations. Such allocation of functions is an example only. In some implementations, the high-speed interface 2508 is coupled to the memory 2504, the display 2316 (e.g., through a graphics processor or accelerator), and to the high-speed expansion ports 2310, which may accept various expansion cards (not shown). In the implementation, the low-speed interface 2312 is coupled to the storage device 2506 and the low-speed expansion port 2314. The low-speed expansion port 2314, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 2500 may be implemented in a number of different forms, as shown in the figure. For example, it may be implemented as a standard server 2320, or multiple times in a group of such servers. In addition, it may be implemented in a personal computer such as a laptop computer 2322. It may also be implemented as part of a rack server system 2324. Alternatively, components from the computing device 2500 may be combined with other components in a mobile device (not shown), such as a mobile computing device 2350. Each of such devices may contain one or more of the computing device 2500 and the mobile computing device 2350, and an entire system may be made up of multiple computing devices communicating with each other.

The mobile computing device 2350 includes a processor 2352, a memory 2364, an input/output device such as a display 2354, a communication interface 2366, and a transceiver 2368, among other components. The mobile computing device 2350 may also be provided with a storage device, such as a micro-drive or other device, to provide additional storage. Each of the processor 2352, the memory 2364, the display 2354, the communication interface 2366, and the transceiver 2368, are interconnected using various buses, and several of the components may be mounted on a common motherboard or in other manners as appropriate.

The processor 2352 can execute instructions within the mobile computing device 2350, including instructions stored in the memory 2364. The processor 2352 may be implemented as a chipset of chips that include separate and multiple analog and digital processors. The processor 2352 may provide, for example, for coordination of the other components of the mobile computing device 2350, such as control of user interfaces, applications run by the mobile computing device 2350, and wireless communication by the mobile computing device 2350.

The processor 2352 may communicate with a user through a control interface 2358 and a display interface 2356 coupled to the display 2354. The display 2354 may be, for example, a TFT (Thin-Film-Transistor Liquid Crystal Display) display or an OLED (Organic Light Emitting Diode) display, or other appropriate display technology. The display interface 2356 may include appropriate circuitry for driving the display 2354 to present graphical and other information to a user. The control interface 2358 may receive commands from a user and convert them for submission to the processor 2352. In addition, an external interface 2362 may provide communication with the processor 2352, so as to enable near area communication of the mobile computing device 2350 with other devices. The external interface 2362 may provide, for example, for wired communication in some implementations, or for wireless communication in other implementations, and multiple interfaces may also be used.

The memory 2364 stores information within the mobile computing device 2350. The memory 2364 can be implemented as one or more of a computer-readable medium or media, a volatile memory unit or units, or a non-volatile memory unit or units. An expansion memory 2374 may also be provided and connected to the mobile computing device 2350 through an expansion interface 2372, which may include, for example, a SIMM (Single In Line Memory Module) card interface. The expansion memory 2374 may provide extra storage space for the mobile computing device 2350, or may also store applications or other information for the mobile computing device 2350. Specifically, the expansion memory 2374 may include instructions to carry out or supplement the processes described above, and may include secure information also. Thus, for example, the expansion memory 2374 may be provided as a security module for the mobile computing device 2350, and may be programmed with instructions that permit secure use of the mobile computing device 2350. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier and, when executed by one or more processing devices (for example, processor 2352), perform one or more methods, such as those described above. The instructions can also be stored by one or more storage devices, such as one or more computer- or machine-readable mediums (for example, the memory 2364, the expansion memory 2374, or memory on the processor 2352). In some implementations, the instructions can be received in a propagated signal, for example, over the transceiver 2368 or the external interface 2362.

The mobile computing device 2350 may communicate wirelessly through the communication interface 2366, which may include digital signal processing circuitry where necessary. The communication interface 2366 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 2368 using a radio-frequency. In addition, short-range communication may occur, such as using a Bluetooth®, Wi-Fi™, or other such transceiver (not shown). In addition, a GPS (Global Positioning System) receiver module 2370 may provide additional navigation- and location-related wireless data to the mobile computing device 2350, which may be used as appropriate by applications running on the mobile computing device 2350.

The mobile computing device 2350 may also communicate audibly using an audio codec 2360, which may receive spoken information from a user and convert it to usable digital information. The audio codec 2360 may likewise generate audible sound for a user, such as through a speaker, e.g., in a handset of the mobile computing device 2350. Such sound may include sound from voice telephone calls, may include recorded sound (e.g., voice messages, music files, etc.) and may also include sound generated by applications operating on the mobile computing device 2350.

The mobile computing device 2350 may be implemented in a number of different forms, as shown in the figure. For example, it may be implemented as a cellular telephone 2380. It may also be implemented as part of a smart-phone 2382, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

### EXEMPLIFICATION

The following Examples illustrate the synthesis of a representative number of compounds, characterization of parameters implicated in assay development, and the use of these compounds in the measurement of samples in, for example, heterogeneous competitive assay. The Examples are intended to illustrate but not to limit the disclosure.

### Example 1

This study was designed to evaluate the analytical performance of the Atellica® IM Serum Neurofilament Light Chain (sNfL) Assay of the present disclosure for the quantitation of NfL in blood.

### Assay Principle

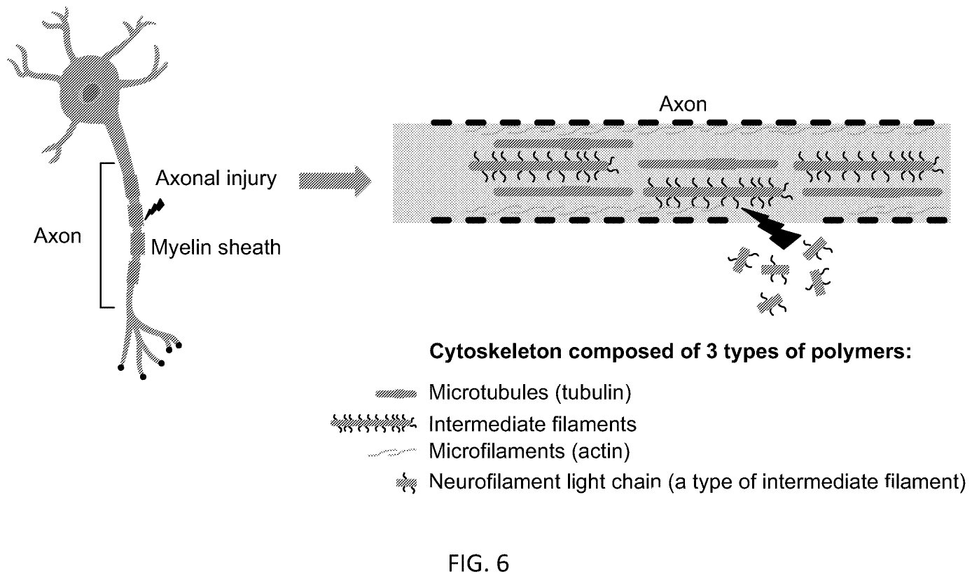

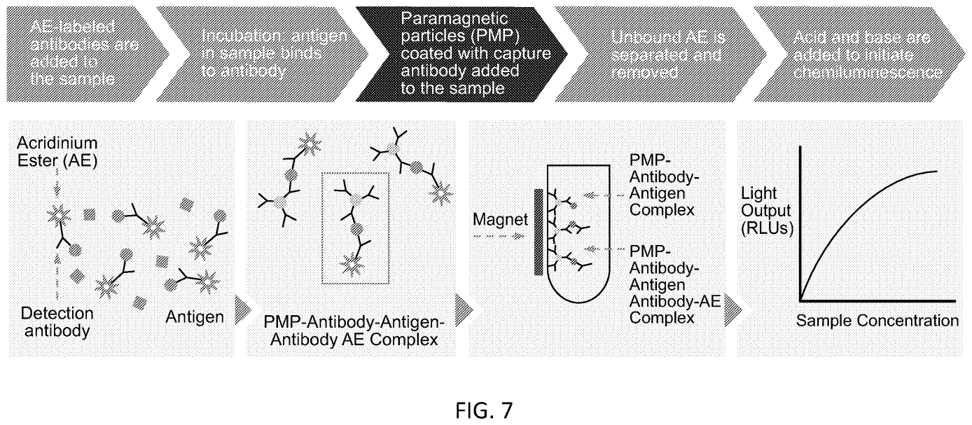

Following axonal damage, NfL is released into cerebrospinal fluid (CSF) and, to a lesser extent, into blood circulation (FIG. 6). The chemiluminescent moiety, acridinium ester (AE), has been pioneering the development of novel chemiluminescence-based immunoassays. The Atellica sNfL Assay is a fully automated 2-step sandwich immunoassay using acridinium ester chemiluminescent technology. The assay employs two anti-sNfL antibodies. The first antibody, in the Lite Reagent, is a mouse monoclonal anti-sNfL antibody labeled with acridinium ester. The second antibody is a biotinylated mouse monoclonal anti-sNfL antibody that is bound to streptavidin-coated paramagnetic microparticles in the Solid Phase. A direct relationship exists between the amount of sNfL present in the patient sample and the amount of relative light units (RLUs) detected by the system. FIG. 7 depicts an Atellica IM sNfL Assay architecture suitable for use in accordance with the present disclosure.

### Results

Precision Studies: Precision runs were performed according to CLSI EP05-A3. Two runs were performed each day for 20 nonconsecutive test days, with a minimum of 2 hours between runs. Samples were tested in duplicate producing a total of n=80 for each system/lot combination. For each assay, one representative system/lot combination result across all lot and system combinations tested is shown. A panel of six native and one contrived human serum/EDTA plasma samples were tested as indicated in Table 3. Samples were frozen (stored at ≤−20° C.) in aliquots prior to the start of the study. Each testing day, new aliquots were thawed and used for each run. Calibrators and QC materials were handled according to the manufacturer's instructions.

TABLE 3

| | Within- |
|---|---|
| | laboratory |
| Repeatability | Precision |

| Specimen | Mean | SD | CV | SD | CV |
|---|---|---|---|---|---|
| Type | (pg/mL) | (pg/mL) | (%) | (pg/mL) | (%) |
| SERUM1 | 4.4 | 0.18 | 4.1 | 0.22 | 5.0 |
| SERUM2 | 9.9 | 0.22 | 2.2 | 0.30 | 3.0 |
| SERUM3 | 16.2 | 0.25 | 1.5 | 0.30 | 1.9 |
| SERUM4 | 24.5 | 0.36 | 1.5 | 0.51 | 2.1 |
| SERUM5 | 85.8 | 0.88 | 1.0 | 1.45 | 1.7 |
| SERUM6 | 227.2 | 2.65 | 1.2 | 3.90 | 1.7 |
| EDTA | 51.0 | 0.65 | 1.3 | 0.89 | 1.7 |

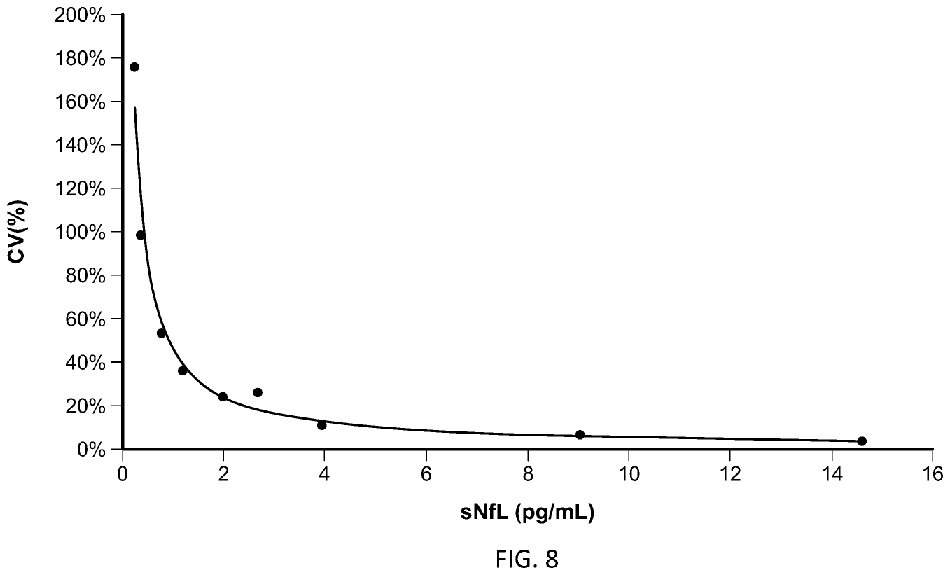

Detection Capability: The limit of Blank (LoB), limit of detection (LoD), and limit of quantitation (LoQ) were determined following CSLI EP17-A2 (Table 4). LoB was calculated non-parametrically using measurements of 160 replicates obtained from four sNfL depleted human serum samples. LoD was evaluated using low level samples and plotting the within-lab standard deviation (SD) versus the analyte concentration. LoD was defined as the lowest analyte concentration that could de differentiated from the LoB with 95% confidence. LoQ was evaluated using a total of 480 measurements from the same low-level samples used to evaluate LoD and was defined as the concentration at which the within-lab CV was ≤20% (FIG. 8).

TABLE 4
Detection limits for the Atellica sNfL Assay.

| | Observed Limit, sNfL | Target Limit, sNfL |
|---|---|---|
| Attribute | (pg/mL) | (pg/mL) |
| LoB | 0.5 | <LoD |
| LoD | 1.3 | ≤2 |
| LoQ | 2.4 | 2.5 |

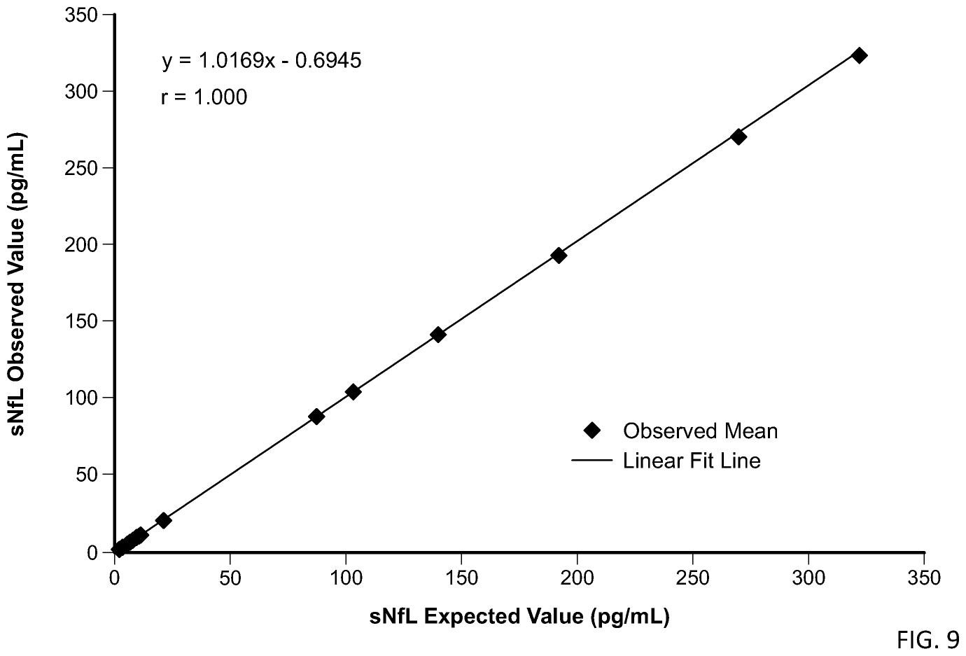

Assay linearity: Linearity of the assay was assessed following CLSI-EP-06-ED2. High and low analyte samples were mixed in various proportions to create eighteen samples spanning the assay range (2.5-300.0 μg/mL). Each sample was assayed in replicates of n=5. Expected values were determined from the calculated concentrations of the low and high samples and their known ratios at each level and plotted against the observed mean values (n=5) for each sample (FIG. 9). The assay was considered linear if the deviation from the expected value was ≤10%.

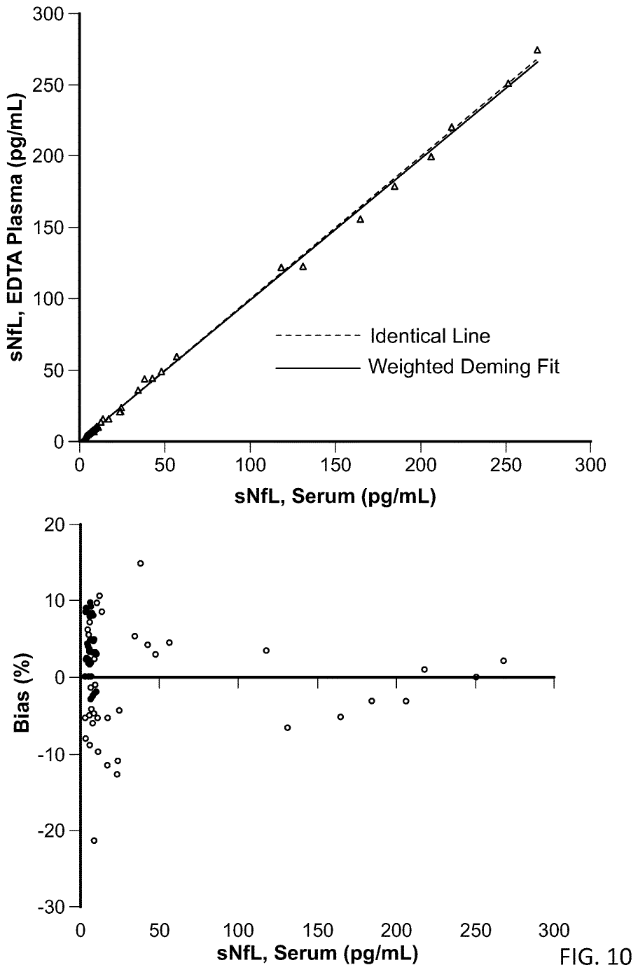

Specimen equivalency: The Atellica sNfL Assay is intended to be used for measurement of neurofilament light chain in human serum and plasma. The specimen equivalence study assessed the correlation between the serum and plasma results and included a total of 69 native and 6 contrived paired serum SST/plasma EDTA. The contrived samples were native paired serum SST/plasma EDTA spiked with <5% cerebral spinal fluid (CSF) from Amyotrophic Lateral Sclerosis (ALS) donors. FIG. 10 demonstrates that no significant bias was observed between serum and EDTA across the sample range tested (2.8-268.4 μg/mL).

Interference and Cross-reactivity studies: Endogenous and exogenous interference was tested following CLSI EP7-A2. No significant interference was observed for hemoglobin, bilirubin, triglycerides, HAMA, rheumatoid factor, biotin (3500 ng/mL), or for a panel of common drugs. All biases were <10%. No significant cross-reactivity of the sNfL assay for neurofilament medium (NfM) and neurofilament heavy (NfH) chains was observed.

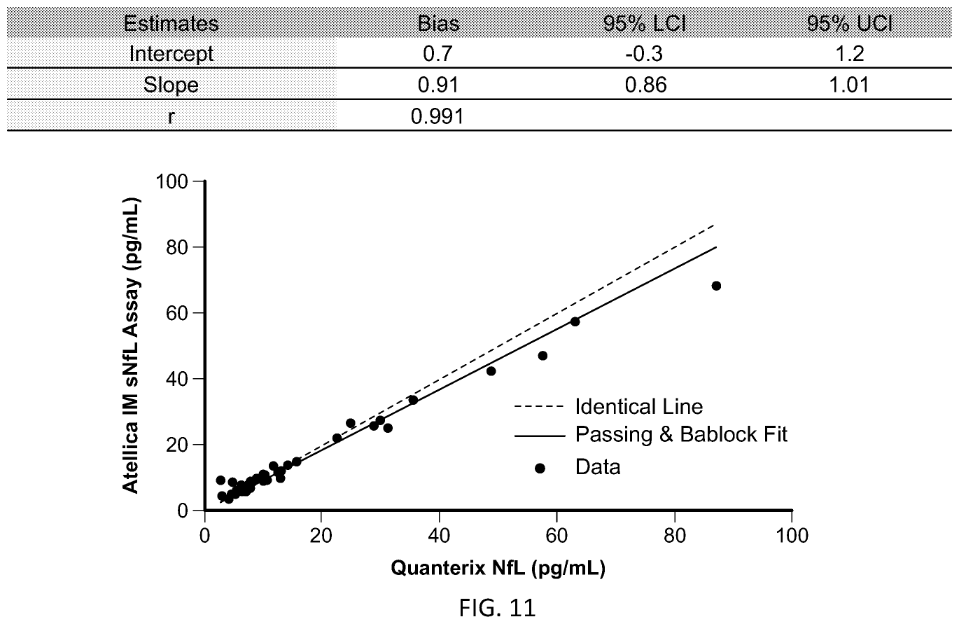

Method comparison (MC) studies were performed according to CLSI EP09-A3 using the Quanterix SIMOA NfL assay. Frozen serum samples (n=48) from multiple sclerosis patients enrolled in the ASCLEPIOS I clinical trial were used. Linear regression statistics were calculated using Passing and Bablock regression analysis. Results show good correlation (FIG. 11).

### CONCLUSIONS

Results indicate that the Atellica sNfL Assay demonstrated analytical performance capable of measuring NfL in serum and EDTA plasma with good accuracy and precision. No significant interference observed from common endogenous and exogenous substances that were tested. No significant cross-reactivity observed with NfM or NfH. Good concordance was observed between the Atellica sNFL Assay and the Quanterix NfL assay.

### Example 2: Production of Antibody Fragments

To produce the immunoglobulin fragments, antibody was digested by the enzyme Ficin to yield two F(ab) fragments (each having an approximate weight of 50 kDa) and an Fc fragment.

To produce the fragment, mouse monoclonal antibody (Mab) was warmed at 37° C. for an hour. Ficin F6008-100UN/086K770 was dissolved at 10 mg/ml, pH7, and then added to the Mab at a 1:30 mass ratio (270 mg Mab/30) 10 mg/ml=0.9 ml Ficin. L-cystein (121.6)(Aldrich 168149/BCBW3951) was then dissolved at 10 mg/ml, pH, which was spiked into the mixture to 1 mM. N-ethylmaleimide (NEM, Sigma E3871/SLBW6111) at 0/1 M, pH 7, was then added to the mixture stirring for 30 min at room temperature (21° C.). The mixture was then placed at 4° C. overnight to produce a digest mixture.

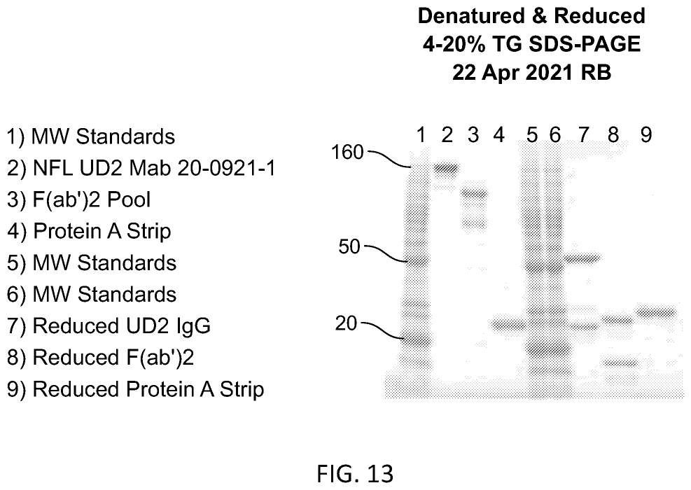

The digest mixture was diluted 1:1 volumetrically with 1.5M glycine-2M NaCl, pH 8.6, and passed over an equilibrated 1×18 protein A Millipore ProSep-vA 113115827/R7AA65871 separation medium. The flow was captured through a 40 ml, pH 8.6 wash. The flow through pool (F/T pool) was 30K concentrated and purified over a 26/70 Superdex-200 column into 0.1 M NaPO₄-150 mM NaCl-5 mM EDTA, pH7.4 (PBSE7.4). The SDS-PAGE analysis is provided in FIG. 13.

### Example 3: Formation of Acridinium-BSA-PEG-F(Ab) Conjugate

Bovine serum albumin was labelled with a 5 to 25 excess of acridinium ester comprising a reactive functional group such as TSPAE-NHS having the structure:

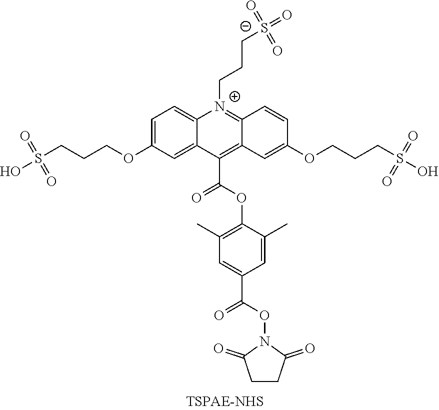

The reacted mixtures were purified over 2.3/18 cm G-25 fine filtration medium into 0.1 M NaPO₄ —150 mM NaCl-0.1% Tween-20, pH 7.4 (PBST7.4) and concentrated to a desired amount of protein.

The carrier protein/acridinium complex was then reacted to form specific antibody/antibody fragment conjugated protein/acridinium complexes. These two were coupled using linking molecules such as polyethylene molecules (e.g., RFG-(OCH₂CH₂)ₙ—RFG, wherein n is from, for example 1-15 and RFG is independently a reactive functional group for forming a conjugate with a protein). The complex was then purified using chromatographic procedures.

Specific conjugates formed using this procedure include neurofilament light chain antibody fragments (Nfl Mab Fab′) conjugated to TSPAE (AE) through pegylation (PEG such as PEG₄ (n=4)) and bovine serum albumin carrier protein (Nfl Mab Fab′-PEG₄-mBSA-AE). The conjugate having 25× excess acridinium ester per carrier protein, and antibody fragments for the UD2 clone of neurofilament light chain is referred to herein as 25λ UD2-Fab′-PEG₄-m-BSA-AE.

### Example 4: Immunoassay Measurements

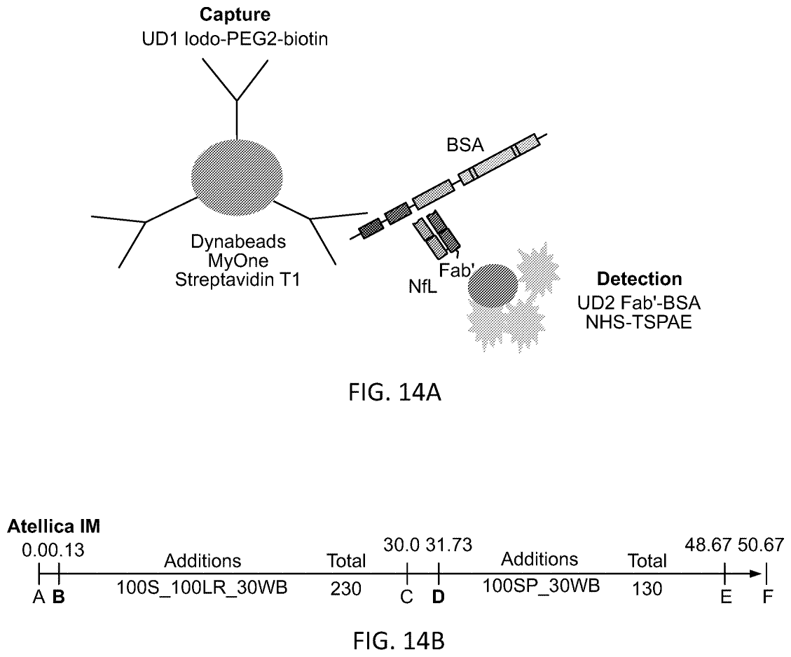

A fully automated 2-step sandwich assay was performed using a Siemens ATELLICA® immunoassay and the conjugates of the present disclosure. The assay employs two anti-sNfl antibodies. The first antibody, in the Lite Reagent is 25×-UD2-Fab-PEG₄-m-BSA-AE. The second antibody is a biotinylated mouse monoclonal anti-sNfl antibody (UD1) bound to a streptavidin coated paramagnetic microparticle in the solid phase (5× UD1-Iodo-PEG₂-Biotin). An exemplary assay format used in the experiments and schematic is shown in FIGS. 14A and 14B.

The Lite Reagent immunoassay composition used is provided in Tables 5 and 6 (Table 6 provides the sNfL Lite Reagent Buffer formulation).

TABLE 5

| Component | Concentration | | Function |
|---|---|---|---|
| sNfL Lite Reagent Buffer | 1 | L/L | Buffer |
| 25X UD2-Fab′-PEG₄-m-BSA-AE | 0.35 | mg/L | Lite Reagent |
| | | | Concentrate |

TABLE 6

| Component | Function |
|---|---|
| HEPES, Acid | Buffer Salt |
| HEPES, Sodium Salt | Buffer Salt |
| Sodium Chloride | Ionic Strength |
| EDTA, Disodium Salt | Protease |
| | Inhibitor |
| Silwet-L 7604 | Surfactant |
| Sodium Azide | Preservative |
| Micr-O-Protect | Preservative |
| Mouse IgG | Blocker |
| PolyMak 33 | Heterophilic |
| | Antibody |
| | Blocker |
| Bovine Gamma Globulin (BgG) | Blocker |
| Bovine Serum Albumin, Sulfhydryl Modified | Blocker |

pH: 7.05-7.15

The Solid Phase immunoassay composition used is provided in Tables 7 and 8 (Table 8 provides the components of the sNfL Solid Phase Buffer).

TABLE 7

| Component | Concentration | Function |
|---|---|---|
| sNfL Solid Phase Buffer | 1 L/L | Buffer |
| 5X UD1-lodo-PEG₂-Biotin | 0.02 mg/mg | Solid Phase (Capture) |
| | particle | Conjugate |
| Dynabeads MyOne Streptavidin | 0.5 mg | Solid Phase Particles |
| T1 | particle/mL | |

TABLE 8

| Component | Function |
|---|---|
| HEPES, Acid | Buffer Salt |
| HEPES, Sodium Salt | Buffer Salt |
| Sodium Chloride | Ionic Strength |
| EDTA, Disodium Salt | Protease |
| | Inhibitor |
| Silwet-L 7604 | Surfactant |
| Sodium Azide | Preservative |
| Micr-O-protect | Preservative |
| Bovine Gamma Globulin (BgG) | Blocker |
| Bovine Serum Albumin, Sulfhydryl Modified | Blocker |

pH: 7.05-7.15

The assay was performed as follows:
a) 100 μL of sample (or standard) was added to a cuvette;
b) 100 μL of lite reagent was added to the sample and incubated for a desired time period and temperature (e.g., for 32 minutes at 37° C.);
c) 100 μL of solid phase was added to the incubated mixture which was again incubated for a desired time period and temperature (e.g., for 17 minutes at 37° C.);
d) the incubated solid phase containing mixture is washed;
e) chemiluminescent triggering reagents were added (300 μL acid/300 μL base) to induce chemiluminescence; and
f) chemiluminescence measured and reported.

FIG. 14B details an example of this addition sequence used (including first and second reagent dispenses and washes), where A illustrates 100 μL sample addition, B indicates the first pass reagent dispense, C indicates a first pass magnetic separation (no wash), D indicates a second reagent dispense, and E indicates a second pass magnetic separation.

Typically, chemiluminescence reporting was performed by identification of the relative light units (RLU) produced following chemiluminescence triggering. Table 9 provides the RLU measured for a series of standards having the identified does of neurofilament light chain present using this assay procedure solid phase, and conjugates.

TABLE 9

| | Dose | |
|---|---|---|
| Sample | (pg/mL) | RLU |
| STD01 | 0 | 1901 |
| STD02 | 10.6 | 12965 |
| STD03 | 28.6 | 30168 |
| STD04 | 64.5 | 66649 |
| STD05 | 137 | 130988 |
| STD06 | 318 | 298172 |
| STD07 | 660 | 553451 |

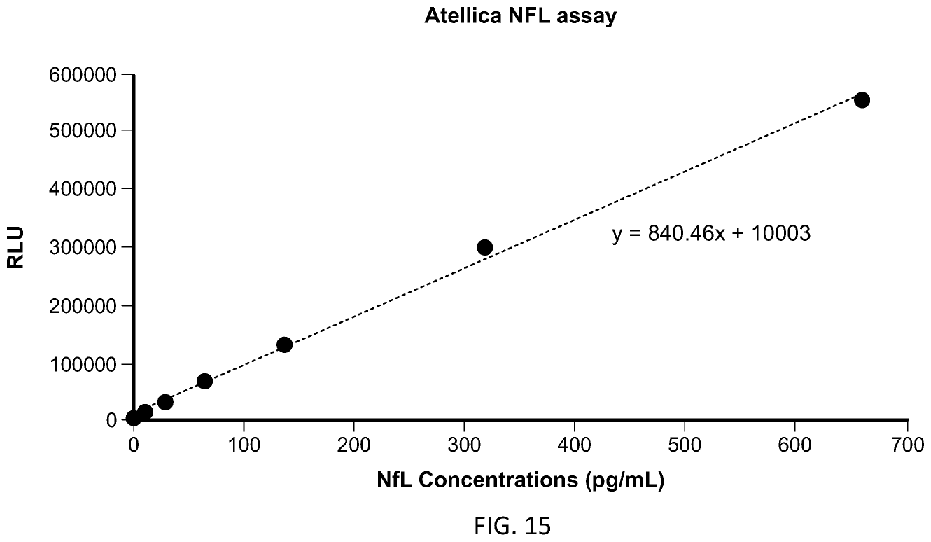

FIG. 15 is a graph of the measured RLU at each NfL concentration including a best fit linear fit (having function RLU=840.46 ([NfL])+10003 shown as the dotted line. These results have a low end slope factor of 836 (RLU(S07-S01)/Dose(S07−S01)-(553451-1901)/(660-0)=836.

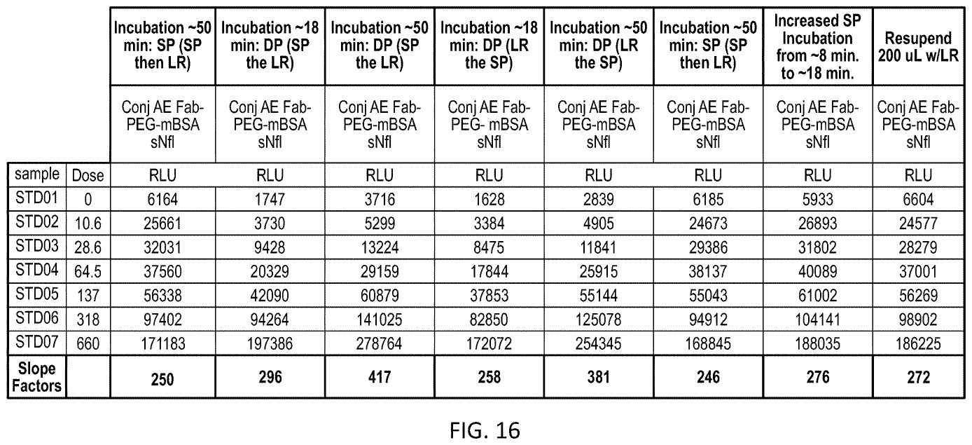

Other assay architectures were unable to yield slope factors greater than 600 including assay architectures without antibody fragments, carrier proteins, and/or linking moieties between the carrier protein and antibody fragment. A high slope factor is needed some highly sensitivity assays in order to produce the high-resolution assay necessary for measurements of certain analytes (such as neurofilament). Table 10 provides the measured RLU and assay formats resulting in assays with slope factors less than 600 resulting in assays with lower resolution taken with various permutations of assay architecture. These measurements were performed using the same conjugate (25×-UD2-Fab-PEG₄-m-BSA-AE). FIG. 16 provides the full data set including the RLU measured for each assay format at each standard.

TABLE 10

| | Slope Factors (between 0 and 660 |
|---|---|
| Assay Format Difference | pg/mL) |
| Incubation ~50 min: SP (SP then LR) | 250 |
| Incubation ~18 min: DP (SP then LR) | 296 |
| Incubation ~50 min: DP (SP then LR) | 417 |
| Incubation ~18 min: DP (LR then SP) | 258 |
| Incubation ~50 min: DP (LR then SP) | 381 |
| Incubation ~50 min: SP (SP then LR) | 246 |
| Incubation ~18 min: SP | 276 |
| Resuspension in 200 μL LR | 272 |

### NON-LIMITING ILLUSTRATIVE EMBODIMENTS

Non-limiting illustrative embodiments are provided below, each of which should be considered to be part of the disclosure of the present application. These embodiments may apply to any embodiment described herein.

Illustrative Embodiment 1. A method for the detection or quantification of an analyte in a sample (e.g., a biological sample such as blood, saliva, serum, a sample derived from a biological sample such as a diluted biological sample) comprising:
(a) mixing the sample with a composition comprising a chemiluminescent label conjugated to a first antibody or antibody fragment that binds to the analyte, wherein the chemiluminescent label is bound to a carrier protein comprising a linker, and the linker is bound to the antibody or antibody fragment;
(b) adding, to the mixture, a particle having a second antibody or antibody fragment that binds to the analyte attached to the surface of the particle;
(c) preparing the mixture to measure chemiluminescence (e.g., by separation of the particle having chemiluminescent acridiniums conjugated to the surface);
(d) triggering chemiluminescence from the preparation; and
(e) detecting the presence or calculating the concentration of said at least one analyte by comparing the amount of chemiluminescence with a standard dose response curve which relates the amount of light emitted to a known concentration of the at least one of the multiple analytes.

Illustrative Embodiment 2. The method according to Illustrative Embodiment 1, the analyte is a neurofilament.

Illustrative Embodiment 3. The method according to Illustrative Embodiment 1 or 2, wherein the sample is blood.

Illustrative Embodiment 4. The method according to any one of Illustrative Embodiments 1-3, wherein the chemiluminescent label is conjugated to a first antibody fragment (e.g., F(ab)).

Illustrative Embodiment 5. The method according to any one of Illustrative Embodiments 1-4, wherein the first antibody or antibody fragment is a mouse monoclonal antibody or fragment thereof (e.g., F(ab)).

Illustrative Embodiment 6. The method according to any one of Illustrative Embodiments 1-5, wherein the linker comprises (or is) polyethylene glycol (PEG).

Illustrative Embodiment 7. The method according to Illustrative Embodiment 6, wherein the polyethelene glycol is from 2-20 (e.g., 2-10, 2-5) ethylene glycol units.

Illustrative Embodiment 8. The method according to any one of Illustrative Embodiments 1-7, wherein the carrier protein is Keyhole Limpet Hemocyanin (KLH), Bovine Serum Albumin (BSA), or cationized BSA.

Illustrative Embodiment 9. The method according to any one of Illustrative Embodiments 1-8, wherein the ratio of acridinium to carrier protein is from 50:1 to 1:1 (e.g., from 30:1 to 1:1, from 25:1 to 5:1) by weight.

Illustrative Embodiment 10. The method according to any one of Illustrative Embodiments 1-9, wherein the chemiluminescent label conjugated to a first antibody or antibody fragment that binds to the analyte was formed by
i) reacting a chemiluminescent acridinium compound comprising a reactive functional group with the carrier protein to label the carrier protein; and
ii) reacting a linking compound (e.g., a compound comprising a linker with reactive functional groups on each end), the first antibody or antibody fragment, and the labelled carrier protein.

Illustrative Embodiment 11. The method according to Illustrative Embodiment 10, wherein the first reacting step occurs in a medium comprising a buffer.

Illustrative Embodiment 12. The method according to Illustrative Embodiment 10 or 11, wherein the chemiluminescent acridinium compound is added in weight excess of the carrier protein (e.g., less than 100× excess or less than 50× excess or less than 40× excess or less than 30× excess or from 5× excess to 25× excess).

Illustrative Embodiment 13. The method according to any one of Illustrative Embodiments 10-12, wherein the chemiluminescent acridinium compound comprising a reactive functional group has the structure:

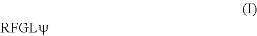

wherein RFG is a reactive functional group for conjugation to a protein,
L is absent (i.e., it is a bond) or a linker optionally comprising a group L^{C} or Z^{L}, and
ψ is a chemiluminescent acridinium comprising the structure:

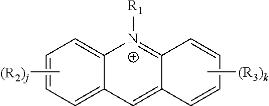

“j” and “k” are independently 0 (e.g., all R₂ groups are hydrogen, all R₃ groups are hydrogen), 1, 2, 3, or 4;
R¹ is hydrogen, —R, —X^{b}, —R^{L}—X^{b}, -L^{C}-R, -L^{C}-X^{b} (e.g., -L₁-X^{b}), —Z, —R^{L}—Z, -L^{C}-Z (e.g., -L₁-Z), or —R^{L}-L^{C}-R^{L}—Z (e.g., —R^{L}-L₁-R^{L}—Z);
R₂ and R₃ are independently selected at each occurrence from hydrogen, —R, an electron donating group, —X^{c}, —R^{L}—X^{c}, -L^{C}-X^{c} (e.g., -L₁-X^{c}), and —Z; wherein two vicinal R₂ or R₃ groups may together form a fused cyclic group (e.g., 5-7 membered fused aryl or heteroaryl group, 5-7 membered fused heterocyclic group) and wherein R₂ or R₃ may comprise a linkage to an imaging agent such as a fluorophore (e.g., rhodamine);
L^{C} is a divalent C₁₋₃₅ alkyl, alkenyl, alkynyl, aryl, or arylalkyl radical, optionally substituted (e.g., with 1 to 20 heteroatoms, with 1-20 substituents);
Z^{L} is a zwitterionic linker group having the structure:

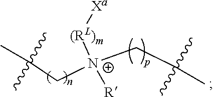

“m” is 0 (i.e. it is a bond) or 1;
“n” and “p” are independently at each occurrence an integer from 0 (i.e. it is a bond) to 10;
Z is a zwitterionic group independently at each occurrence has the structure:

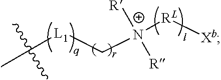

“q” and “l” are independently 0 or 1;
“r” is independently an integer from 0 to 10 (e.g., from 1 to 10, 1, 2, 3, 4, 5, 6, 7, 8, 9, 10);
X^{a} and X^{b} are independently at each occurrence an anionic group;
X^{c} is a protonated anionic group;
L₁ is independently at each occurrence —O—, —S—, —NH—, —N(R^{N})—, —(CH₂)₁₋₁₀—, —S(═O)₁₋₂—, —C═C—, —C═C—(CH₂)₁₋₃—, —C(O)—, —O—C(O)—, —C(O)—(CH₂)₁₋₄—, —(CH₂)₁₋₄—C(O)—, —C(O)—O—, —C(O)—N(R^{N})—, —C(O)—NH—, —N(R^{N})—C(O)—, —NH—C(O)—, —C(O)—N(R^{N})—(CH₂)₁₋₃—, —(CH₂)₁₋₃—C(O)—N(R^{N})—, —(CH₂)₁₋₃—N(R^{N})—C(O)—, —NH—S(O)₁₋₂—, —N(R^{N})—S(O)₁₋₂—, —S(O)₁₋₂—N(R^{N})—, —S(O)₁₋₂—NH—, —(CH₂)₁₋₃—NH—S(O)₁₋₂—, —(CH₂)₁₋₃—N(R^{N})—S(O)₁₋₂—, —(CH₂)₁₋₃—S(O)₁₋₂—N(R^{N})—, —(CH₂)₁₋₃—S(O)₁₋₂—NH—, —O—(CH₂)₁₋₄—, —(CH₂)₁₋₄—O—, —S—(CH₂)₁₋₄—, —(CH₂)₁₋₄—S—, —NH—(CH₂)₁₋₄—, —N(R^{N})—(CH₂)₁₋₄—, —(CH₂)₁₋₄—N(R^{N})—, —(OCH₂)₁₋₁₀—, —(CH₂O)₁₋₁₀—, —(OCH₂CH₂)₁₋₁₀—, or —(CH₂CH₂O)₁₋₁₀—;
R^{L} is independently at each occurrence a C₁₋₂₀ bivalent hydrocarbon radical (e.g., alkyl, alkenyl, aryl, phenyl, mono alkyl substituted phenyl, di alkyl substituted phenyl, alkynyl, arylalkyl), optionally having one or more (e.g., 1-10, 1-5) points of substitution (e.g., with 1-10 heteroatoms, with 1-10 substituents);
R is independently at each occurrence hydrogen or C₁₋₃₅ hydrocarbon (e.g., alkyl, alkenyl, alkynyl, or aralkyl) radical, optionally having one or more (e.g., 1-20, 1-10, 1-5) points of substitution (e.g., with 1-20 heteroatoms, with 1-20 substituents);
R′ and R″ are independently at each occurrence hydrogen or a C₁₋₁₀ alkyl;
R^{N} is independently at each occurrence from hydrogen or C₁₋₅ alkyl (e.g., methyl, ethyl, propyl); and
R′ is hydrogen or a C₁₋₁₀ alkyl; or
a salt thereof (e.g., a halide salt such as a chloride salt, a sulfonate salt such as a halosulfonate salt, a haloalkyl sulfonate salt a fluoroalkyl sulfonate salt, a carboxylate salt such as a haloalkyl carboxylate salt, fluoroalkyl carboxylate salt).

Illustrative Embodiment 14. The method according to any one of Illustrative Embodiments 10-13, wherein the chemiluminescent acridinium comprising a reactive functional group has the structure of formula (Ia):

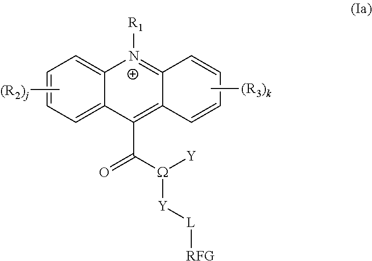

wherein Ω is O or N;
Y is selected from —R or —R^{L}—Z, or in the case where Q is O then Y is absent; and
Y′ is either absent (i.e. it is a bond), or is selected from -L₁-, —R^{L}-R^{L}-L₁-, -L₁-L₁-, -L₁-R^{L}—, -L₁-R^{L}-L₁, and —R^{L}-L₁-R^{L}—.

Illustrative Embodiment 15. The method according to any one of Illustrative Embodiments 10-14, wherein the chemiluminescent acridinium comprising a reactive functional group has the structure of formula (Ia) or (Ib):

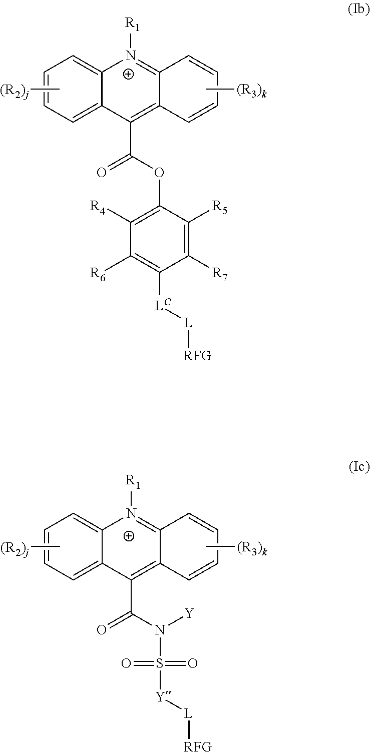

wherein R₄-R₇ are independently hydrogen, an electron donating group, or C₁₋₃₅ alkyl, alkenyl, alkynyl, aryl, alkoxy, alkylthio, or amino; and
Y″ is either absent (i.e., it is a bond) or -L^{C}-, -L₁-, —R^{L} or R^{L}-L-.

Illustrative Embodiment 16. The method according to any one of Illustrative Embodiments 13-15, wherein L is a C₁-C₅ alkylene.

Illustrative Embodiment 17. The method according to any one of Illustrative Embodiments 10-16, wherein said chemiluminescent acridinium comprising a reactive functional group is an N-succinimidyl ester.

Illustrative Embodiment 18. The method according to any one of Illustrative Embodiments 13-17, wherein R₂ and R₃ are independently hydrogen —X^{c}, —R^{L}—X^{c}, -L^{C}-X^{c} (e.g., -L₁-X^{c}) and at least one (e.g. both) of R₂ or R₃ is not hydrogen.

Illustrative Embodiment 19. The method according to any one of Illustrative Embodiments 13-17, wherein R₂ and R₃ are independently alkoxy (e.g., C₁-C₄ alkoxy) substituted with —C(O)OH, —SO₂OH), —OSO₂OH), —OP(O)(OR^{P})OH, —OH, or combinations thereof.

Illustrative Embodiment 20. The method according to any one of Illustrative Embodiments 10-19, wherein said chemiluminescent acridinium comprising a reactive functional group is TSPAE-NHS:

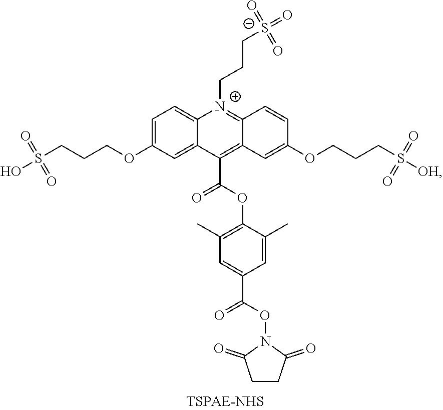

or a salt thereof.

Illustrative Embodiment 21. The method according to any one of Illustrative Embodiments 1-20, wherein the second antibody or antibody fragment is a biotinylated antibody or antibody fragment.

Illustrative Embodiment 22. The method according to any one of Illustrative Embodiments 1-21, wherein the particle is streptavidin coated.

Illustrative Embodiment 23. The method according to any one of Illustrative Embodiments 1-22, wherein said detecting step can detect a difference in concentration of less than 5 μg/mL (e.g., less than 4 μg/mL, from 1 μg/mL to 5 μg/mL, from 2-5 μg/mL, from 2-4 μg/mL, from 3-4 μg/mL, from 2-3 μg/mL).

Illustrative Embodiment 24. The method according to any one of Illustrative Embodiments 1-23, wherein said preparing step comprises separating the particle from the mixture and the chemiluminescence is triggered from the particle or the separated mixture.

Illustrative Embodiment 25. The method according to any one of Illustrative Embodiments 1-24, wherein the method further comprises incubating the mixture prior to adding the particle.

Illustrative Embodiment 26. The method according to Illustrative Embodiment 25, wherein said incubating comprises heating the mixture for more than 30 minutes (e.g., from 30 minutes to 120 minutes, from 30 minutes to 60 minutes).

Illustrative Embodiment 27. The method according to any one of Illustrative Embodiments 1-26, wherein the method further comprises incubating the mixture after adding the particle.

Illustrative Embodiment 28. The method according to Illustrative Embodiment 27, wherein said incubating comprises heating the mixture for less than 30 minutes (e.g., from 10 minutes to 30 minutes, from 10 minutes to 20 minutes).

Illustrative Embodiment 29. An immunoassay composition comprising a chemiluminescent label conjugated to a first antibody fragment that binds to nerofilament, wherein the chemiluminescent label is bound to a carrier protein comprising a polyethylene glycol linker (e.g., PEG₂-PEG₁₅ such as PEG₄), and the linker is bound to the antibody fragment; and a carrier or excipient.

Illustrative Embodiment 30. The immunoassay composition according to Illustrative Embodiment 29, wherein said composition further comprises a buffer.

Illustrative Embodiment 31. The immunoassay composition according to Illustrative Embodiment 29 or 30, wherein said carrier protein is bovine serum albumin.

Illustrative Embodiment 32. The immunoassay composition according to any one of Illustrative Embodiments 29-31, wherein the weight ratio of said chemiluminescent acridinium to carrier protein is from 50:1 to 1:1 (e.g., from 30:1 to 1:1, from 25:1 to 5:1) by weight.

Illustrative Embodiment 33. A solid particle coated with streptavidin conjugated to biotinylated mouse anti neurofilament antibody optionally through a linker (e.g., PEG, Iodo-PEG), wherein the anti neurofilament antibody is bound neurofilament, and the bound neurofilament is further bound to a monoclonal mouse antibody fragment linked to a carrier protein conjugated to one or more chemiluminescent acridiniums.

Illustrative Embodiment 34. The solid particle according to Illustrative Embodiment 33, wherein the monoclonal mouse antibody fragment is linked to the carrier protein through a linker (e.g., PEG).

All references including patent applications and publications cited herein are incorporated herein by reference and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The embodiments described herein are offered by way of example only, and the invention is to be limited only by the terms of the appended claims, along with the full scope of equivalents to which such claims are entitled.

### EQUIVALENTS

Those skilled in the art will recognize or be able to ascertain using no more than routine experimentation, many equivalents to the specific embodiments of the disclosure described herein. The scope of the present disclosure is not intended to be limited to the above Description.

1. A method for the detection or quantification of an analyte in a sample (e.g., a biological sample such as blood, saliva, serum, a sample derived from a biological sample such as a diluted biological sample) comprising:
(a) mixing the sample with a composition comprising a chemiluminescent label conjugated to a first antibody or antibody fragment that binds to the analyte, wherein the chemiluminescent label is bound to a carrier protein comprising a linker, and the linker is bound to the antibody or antibody fragment;
(b) adding, to the mixture, a particle having a second antibody or antibody fragment that binds to the analyte attached to the surface of the particle;
(c) preparing the mixture to measure chemiluminescence (e.g., by separation of the particle having chemiluminescent acridiniums conjugated to the surface);
(d) triggering chemiluminescence from the preparation; and
(e) detecting the presence or calculating the concentration of said at least one analyte by comparing the amount of chemiluminescence with a standard dose response curve which relates the amount of light emitted to a known concentration of the at least one of the multiple analytes.

2. The method according to claim 1, the analyte is a neurofilament.

3. The method according to claim 2, wherein the chemiluminescent label is conjugated to a first antibody fragment (e.g., F(ab)).

4. The method according to claim 3, wherein the first antibody or antibody fragment is a mouse monoclonal antibody or fragment thereof (e.g., F(ab)).

5. The method according to claim 4, wherein the linker comprises (or is) polyethylene glycol (PEG).

6. The method according to claim 5, wherein the polyethelene glycol is from 2-20 (e.g., 2-10, 2-5) ethylene glycol units.

7. The method according to claim 6, wherein the carrier protein is Keyhole Limpet Hemocyanin (KLH), Bovine Serum Albumin (BSA), or cationized BSA.

8. The method according to claim 7, wherein the ratio of acridinium to carrier protein is from 50:1 to 1:1 (e.g., from 30:1 to 1:1, from 25:1 to 5:1) by weight.

9. The method according to claim 8, wherein the chemiluminescent label conjugated to a first antibody or antibody fragment that binds to the analyte was formed by
i) reacting a chemiluminescent acridinium compound comprising a reactive functional group with the carrier protein to label the carrier protein; and
ii) reacting a linking compound (e.g., a compound comprising a linker with reactive functional groups on each end), the first antibody or antibody fragment, and the labelled carrier protein.

10. The method according to claim 9, wherein the chemiluminescent acridinium compound is added in weight excess of the carrier protein (e.g., less than 100× excess or less than 50× excess or less than 40× excess or less than 30× excess or from 5× excess to 25× excess).

11. The method according to claim 10, wherein the chemiluminescent acridinium compound comprising a reactive functional group has the structure: 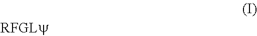
wherein RFG is a reactive functional group for conjugation to a protein,
L is absent (i.e., it is a bond) or a linker optionally comprising a group L^{C} or Z^{L}, and
ψ is a chemiluminescent acridinium comprising the structure: 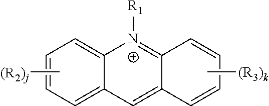
“j” and “k” are independently 0 (e.g., all R₂ groups are hydrogen, all R₃ groups are hydrogen), 1, 2, 3, or 4;
R₁ is hydrogen, —R, —X^{b}, —R^{L}—X^{b}, -L^{C}-R, -L^{C}-X^{b} (e.g., -L₁-X^{b}), —Z, —R^{L}—Z, -L^{C}-Z (e.g., -L₁-Z), or —R^{L}-L^{C}-R^{L}—Z (e.g., —R^{L}-L₁-R^{L}—Z);
R₂ and R₃ are independently selected at each occurrence from hydrogen, —R, an electron donating group, —X^{c}, —R^{L}—X^{c}, -L^{C}-X^{c} (e.g., -L₁-X^{c}), and —Z; wherein two vicinal R₂ or R₃ groups may together form a fused cyclic group (e.g., 5-7 membered fused aryl or heteroaryl group, 5-7 membered fused heterocyclic group) and wherein R₂ or R₃ may comprise a linkage to an imaging agent such as a fluorophore (e.g., rhodamine);
L^{C} is a divalent C₁₋₃₅ alkyl, alkenyl, alkynyl, aryl, or arylalkyl radical, optionally substituted (e.g., with 1 to 20 heteroatoms, with 1-20 substituents);
Z^{L} is a zwitterionic linker group having the structure: 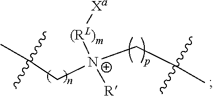
“m” is 0 (i.e. it is a bond) or 1;
“n” and “p” are independently at each occurrence an integer from 0 (i.e. it is a bond) to 10;
Z is a zwitterionic group independently at each occurrence has the structure: 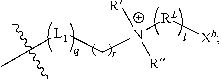
“q” and “/” are independently 0 or 1;
“r” is independently an integer from 0 to 10 (e.g., from 1 to 10, 1, 2, 3, 4, 5, 6, 7, 8, 9, 10);
X^{a} and X^{b} are independently at each occurrence an anionic group;
X^{c} is a protonated anionic group;
L₁ is independently at each occurrence —O—, —S—, —NH—, —N(R^{N})—, —(CH₂)₁₋₁₀—, —S(═O)₁₋₂—, —C═C—, —C═C—(CH₂)₁₋₃—, —C(O)—, —O—C(O)—, —C(O)—(CH₂)₁₋₄—, —(CH₂)₁₋₄—C(O)—, —C(O)—O—, —C(O)—N(R^{N})—, —O(O)—NH—, —N(R^{N})—C(O)—, —NH—C(O)—, —C(O)—N(R^{N})—(CH₂)₁₋₃—, —(CH₂)₁₋₃—C(O)—N(R^{N})—, —(CH₂)₁₋₃—N(R^{N})—C(O)—, —NH—S(O)₁₋₂—, —N(R^{N})—S(O)₁₋₂—, —S(O)₁₋₂—N(R^{N})—, —S(O)₁₋₂—NH—, —(CH₂)₁₋₃—NH—S(O)₁₋₂—, —(CH₂)₁₋₃—N(R^{N})—S(O)₁₋₂—, —(CH₂)₁₋₃—S(O)₁₋₂—N(R^{N})—, —(CH₂)₁₋₃—S(O)₁₋₂—NH—, —O—(CH₂)₁₋₄—, —(CH₂)₁₋₄—O—, —S—(CH₂)₁₋₄—, —(CH₂)₁₋₄—S—, —NH—(CH₂)₁₋₄—, —N(R^{N})—(CH₂)₁₋₄—, —(CH₂)₁₋₄—N(R^{N})—, —(OCH₂)₁₋₁₀—, —(CH₂O)₁₋₁₀—, —(OCH₂CH₂)₁₋₁₀—, or —(CH₂CH₂O)₁₋₁₀—;
R^{L} is independently at each occurrence a C₁₋₂₀ bivalent hydrocarbon radical (e.g., alkyl, alkenyl, aryl, phenyl, mono alkyl substituted phenyl, di alkyl substituted phenyl, alkynyl, arylalkyl), optionally having one or more (e.g., 1-10, 1-5) points of substitution (e.g., with 1-10 heteroatoms, with 1-10 substituents);
R is independently at each occurrence hydrogen or C₁₋₃₅ hydrocarbon (e.g., alkyl, alkenyl, alkynyl, or aralkyl) radical, optionally having one or more (e.g., 1-20, 1-10, 1-5) points of substitution (e.g., with 1-20 heteroatoms, with 1-20 substituents);
R′ and R″ are independently at each occurrence hydrogen or a C₁₋₁₀ alkyl;
R^{N} is independently at each occurrence from hydrogen or C₁₋₅ alkyl (e.g., methyl, ethyl, propyl); and
R′ is hydrogen or a C₁₋₁₀ alkyl; or
a salt thereof (e.g., a halide salt such as a chloride salt, a sulfonate salt such as a halosulfonate salt, a haloalkyl sulfonate salt a fluoroalkyl sulfonate salt, a carboxylate salt such as a haloalkyl carboxylate salt, fluoroalkyl carboxylate salt).

12. The method according to claim 11, wherein the reactive functional group of said chemiluminescent acridinium comprising a reactive functional group is an N-succinimidyl ester.

13. The method according to claim 12, wherein R₂ and R₃ are independently —X^{c}, —R^{L}—X^{c}, -L^{C}-X^{c} (e.g., -L₁-X^{c}).

14. The method according to claim 13, wherein R₂ and R₃ are independently alkoxy (e.g., C₁-C₄ alkoxy) substituted with —C(O)OH, —SO₂OH), —OSO₂OH), —OP(O)(OR^{P})OH, —OH, or combinations thereof.

15. The method according to claim 14, wherein said chemiluminescent acridinium comprising a reactive functional group is 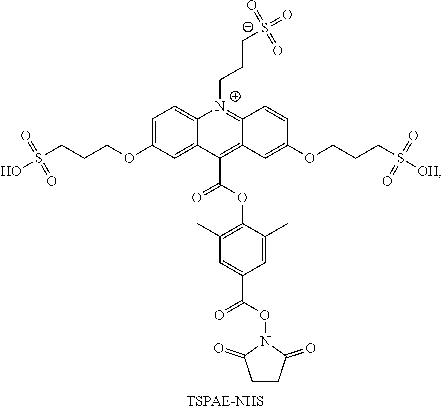
or a salt thereof.

16. The method according to claim 15, wherein the second antibody or antibody fragment is a biotinylated antibody or antibody fragment.

17. The method according to claim 16, wherein said detecting step can detect a difference in concentration of less than 5 μg/mL (e.g., less than 4 μg/mL, from 1 μg/mL to 5 μg/mL, from 2-5 μg/mL, from 2-4 μg/mL, from 3-4 μg/mL, from 2-3 μg/mL).

18. The method according to claim 17, wherein said preparing step comprises separating the particle from the mixture and the chemiluminescence is triggered from the particle or the separated mixture and optionally further comprises incubating the mixture prior to and/or after adding the particle.

19. An immunoassay composition comprising a chemiluminescent label conjugated to a first antibody fragment that binds to nerofilament, wherein the chemiluminescent label is bound to a carrier protein comprising a polyethylene glycol linker (e.g., PEG₂-PEG₁₅ such as PEG₄), and the linker is bound to the antibody fragment; and
a carrier or excipient.

20. A solid particle coated with streptavidin conjugated to biotinylated mouse anti neurofilament antibody optionally through a linker (e.g., PEG, Iodo-PEG), wherein the anti neurofilament antibody is bound neurofilament, and
the bound neurofilament is further bound to a monoclonal mouse antibody fragment linked to a carrier protein conjugated to one or more chemiluminescent acridiniums.
